(19) United States
(12) Patent Application Publication
(10) Pub. No.: US 2022/0033916 A1
(43) Pub. Date: Feb. 3, 2022

(54) METHODS AND COMPOSITIONS FOR EARLY CANCER DETECTION

(71) Applicant: AccuraGen Holdings Limited, George Town (KY)

(72) Inventors: Li WENG, Fremont, CA (US); Malek FAHAM, Burlingame, CA (US); Tobias WITTKOP, San Mateo, CA (US); Yontao LU, Sunnyvale, CA (US); Ling Fung TANG, San Francisco, CA (US)

(21) Appl. No.: 17/405,768

(22) Filed: Aug. 18, 2021

Related U.S. Application Data
(63) Continuation of application No. PCT/US2020/019286, filed on Feb. 21, 2020.
(60) Provisional application No. 62/809,450, filed on Feb. 22, 2019.
(60) Provisional application No. 62/873,113, filed on Jul. 11, 2019.

(51) Int. Cl. C12Q 1/6886

(52) U.S. Cl. CPC C12Q 1/6886

(57) ABSTRACT

Provided herein are methods and systems for detecting non-metastatic cancer in a subject, comprising measuring a total cfDNA fragment size distribution for the plurality of cfDNA nucleic acid molecules of the subject and comparing the total cfDNA fragment size distribution for the plurality of cfDNA nucleic acid molecules of the subject to the total cfDNA fragment size distribution for the plurality of cfDNA nucleic acid molecules from a healthy control.

### CROSS-REFERENCE

This application is a continuation of PCT International Application No. PCT/US2020/019286 filed on Feb. 21, 2020, which claims the benefit of U.S. Provisional Application No. 62/809,450, filed Feb. 22, 2019, and U.S. Provisional Application No. 62/873,113, filed Jul. 11, 2019, each of which are incorporated herein by reference in its entirety.

### BACKGROUND

Early detection of cancer may lead to better outcomes in treatment of cancer patients. Therefore, it is desirable to have methods of detecting cancer early, for example prior to metastasis of the cancer from the primary tumor site. As many cancers show few to no symptoms at these early stages, it may be advantageous to have accurate cancer detection that is minimally invasive and capable of being offered to seemingly healthy individuals during routine health screening.

### SUMMARY

Provided herein are methods of detecting a non-metastatic cancer in a subject. In some embodiments, the method comprises (a) obtaining a sample comprising a plurality of cell-free deoxyribonucleic acid (cfDNA) polynucleotides of the subject; (b) measuring a total cfDNA fragment size distribution for the plurality of cfDNA polynucleotides of the subject; (c) comparing the total cfDNA fragment size distribution for the plurality of cfDNA polynucleotides of the subject to the total cfDNA fragment size distribution for the plurality of cfDNA polynucleotides from a healthy control; and (d) classifying the subject as having an increased risk of non-metastatic cancer when the cfDNA fragment size distribution shows an increase in fragments having sizes of up to 170 bases in the subject compared to the healthy control. Also provided herein are methods of detecting a non-metastatic cancer in a subject comprising: (a) obtaining a sample comprising a plurality of cell-free DNA (cfDNA) polynucleotides of the subject; (b) measuring a total cfDNA fragment size distribution for the plurality of cfDNA polynucleotides of the subject by sequencing; (c) comparing the total cfDNA fragment size distribution for the plurality of cfDNA polynucleotides of the subject to the total cfDNA fragment size distribution for the plurality of cfDNA polynucleotides from a healthy control; and (d) classifying the subject as having an increased risk of non-metastatic cancer when the cfDNA fragment size distribution shows an increase in fragments having sizes of up to 170 bases in the subject compared to the healthy control. In some embodiments, the method further comprises preparation of a single stranded deoxyribonucleic acid (DNA) library from the cfDNA polynucleotides of the subject. In some embodiments, the method further comprises preparation of a double stranded DNA library from the cfDNA polynucleotides of the subject. In some embodiments, the method further comprises: (a) circularizing individual cfDNA polynucleotides of the plurality of cfDNA polynucleotides of the subject to form a plurality of circular polynucleotides; (b) amplifying the circular polynucleotides; (c) sequencing the amplified polynucleotides to produce a plurality of sequencing reads; and (d) determining a length for each individual cfDNA polynucleotide of the plurality of cfDNA polynucleotides of the subject. In some embodiments, the cfDNA fragment size distribution shows at least a 0.5% increase in fragments having sizes of up to 170 bases in the subject compared to the healthy control. In some embodiments, the cfDNA fragment size distribution shows at least a 1% increase in fragments having sizes of up to 170 bases in the subject compared to the healthy control. In some embodiments, the cfDNA fragment size distribution shows at least a 10% increase in fragments having sizes of up to 170 bases in the subject compared to the healthy control. In some embodiments, the method comprises classifying the subject as having an increased risk of non-metastatic cancer when the cfDNA fragment size distribution shows an increase in fragments having sizes of 50 bases to 125 bases in the subject compared to the healthy control. In some embodiments, the cfDNA fragment size distribution shows at least a 0.5% increase in fragments having sizes of 50 bases to 125 bases in the subject compared to the healthy control. In some embodiments, the cfDNA fragment size distribution shows at least a 1% increase in fragments having sizes of 50 bases to 125 bases in the subject compared to the healthy control. In some embodiments, the cfDNA fragment size distribution shows at least a 10% increase in fragments having sizes of 50 bases to 125 bases in the subject compared to the healthy control. In some embodiments, the method comprises classifying the subject as having an increased risk of non-metastatic cancer when the cfDNA fragment size distribution shows an increase in fragments having sizes of 50 bases to 115 bases in the subject compared to the healthy control. In some embodiments, the cfDNA fragment size distribution shows at least a 0.5% increase in fragments having sizes of 50 bases to 115 bases in the subject compared to the healthy control. In some embodiments, the cfDNA fragment size distribution shows at least a 1% increase in fragments having sizes of 50 bases to 115 bases in the subject compared to the healthy control. In some embodiments, the cfDNA fragment size distribution shows at least a 10% increase in fragments having sizes of 50 bases to 115 bases in the subject compared to the healthy control. In some embodiments, the method comprises classifying the subject as having an increased risk of non-metastatic cancer when the cfDNA fragment size distribution shows an increase in fragments having sizes of 50 bases to 105 bases in the subject compared to the healthy control. In some embodiments, the cfDNA fragment size distribution shows at least a 0.5% increase in fragments having sizes of 50 bases to 105 bases in the subject compared to the healthy control. In some embodiments, the cfDNA fragment size distribution shows at least a 1% increase in fragments having sizes of 50 bases to 105 bases in the subject compared to the healthy control. In some embodiments, the cfDNA fragment size distribution shows at least a 10% increase in fragments having sizes of 50 bases to 105 bases in the subject compared to the healthy control. In some embodiments, the subject does not have a diagnosis of metastatic cancer. In some embodiments, the subject has a tumor burden of less than 10%. In some embodiments, the cancer is selected from the group consisting of colon cancer, non-small cell lung cancer, small cell lung cancer, breast cancer, hepatocellular carcinoma, liver cancer, skin cancer, malignant melanoma, endometrial cancer, esophageal cancer, gastric cancer, ovarian cancer, pancreatic cancer, and brain cancer. In some embodiments, the method does not comprise isolating tumor cfDNA polynucleotides from total cfDNA polynucleotides. In some embodiments, the non-metastatic cancer is stage 0, stage 1, stage 2, or stage 3. In some embodiments, the method further comprises recommending administration of a chemotherapy to the subject. In some embodiments, the method further comprises recommending additional cancer monitoring to the subject. In some embodiments, (d) comprises classifying the subject as having an increased risk of non-metastatic cancer when the cfDNA fragment size distribution shows an increase in fragments having sizes of 50 bases up to 170 bases in the subject compared to the healthy control. In some embodiments, the method further comprises enriching the plurality of cfDNA polynucleotides for one or more target sequences. In some embodiments, the plurality of sequencing reads are processed using a sequence(s) of the targeted primer(s) or capture probe(s).

Additionally provided herein are methods of measuring a cfDNA size distribution in a blood sample from a subject. In some embodiments the method comprises (a) obtaining a sample comprising a plurality of cell-free DNA (cfDNA) polynucleotides from the blood sample of the subject; (b) measuring a total cfDNA fragment size distribution for the plurality of cfDNA polynucleotides of the subject; (c) comparing the total cfDNA fragment size distribution for the plurality of cfDNA polynucleotides of the subject to the total cfDNA fragment size distribution for the plurality of cfDNA polynucleotides from a healthy control; and (d) classifying the subject as having an increased risk of non-metastatic cancer when the cfDNA fragment size distribution shows an increase in fragments having sizes of up to 170 bases in the subject compared to the healthy control. In some embodiments, the plurality of cfDNA polynucleotides comprises less than 10% circulating tumor DNA (ctDNA). In some embodiments, the plurality of cfDNA polynucleotides comprises less than 5% circulating tumor DNA (ctDNA). In some embodiments, the plurality of cfDNA polynucleotides comprises less than 2% circulating tumor DNA (ctDNA). In some embodiments, the plurality of cfDNA polynucleotides comprises less than 1% circulating tumor DNA (ctDNA). In some embodiments, the method further comprises preparation of a single stranded DNA library from the cfDNA polynucleotides of the subject. In some embodiments, the method further comprises preparation of a double stranded DNA library from the cfDNA polynucleotides of the subject. In some embodiments, the method further comprises: (a) circularizing individual cfDNA polynucleotides of the plurality of cfDNA polynucleotides of the subject to form a plurality of circular polynucleotides; (b) amplifying the circular polynucleotides; (c) sequencing the amplified polynucleotides to produce a plurality of sequencing reads; and (d) determining a length for each individual cfDNA polynucleotide of the plurality of cfDNA polynucleotides of the subject. In some embodiments, the cfDNA fragment size distribution shows at least a 0.5% increase in fragments having sizes of up to 170 bases in the subject compared to the healthy control. In some embodiments, the cfDNA fragment size distribution shows at least a 1% increase in fragments having sizes of up to 170 bases in the subject compared to the healthy control. In some embodiments, the cfDNA fragment size distribution shows at least a 10% increase in fragments having sizes of up to 170 bases in the subject compared to the healthy control. In some embodiments, the method comprises classifying the subject as having an increased risk of non-metastatic cancer when the cfDNA fragment size distribution shows an increase in fragments having sizes of 50 bases to 125 bases in the subject compared to the healthy control. In some embodiments, the cfDNA fragment size distribution shows at least a 0.5% increase in fragments having sizes of 50 bases to 125 bases in the subject compared to the healthy control. In some embodiments, the cfDNA fragment size distribution shows at least a 1% increase in fragments having sizes of 50 bases to 125 bases in the subject compared to the healthy control. In some embodiments, the cfDNA fragment size distribution shows at least a 10% increase in fragments having sizes of 50 bases to 125 bases in the subject compared to the healthy control. In some embodiments, the method comprises classifying the subject as having an increased risk of non-metastatic cancer when the cfDNA fragment size distribution shows an increase in fragments having sizes of 50 bases to 115 bases in the subject compared to the healthy control. In some embodiments, the cfDNA fragment size distribution shows at least a 0.5% increase in fragments having sizes of 50 bases to 115 bases in the subject compared to the healthy control. In some embodiments, the cfDNA fragment size distribution shows at least a 1% increase in fragments having sizes of 50 bases to 115 bases in the subject compared to the healthy control. In some embodiments, the cfDNA fragment size distribution shows at least a 10% increase in fragments having sizes of 50 bases to 115 bases in the subject compared to the healthy control. In some embodiments, the method comprises classifying the subject as having an increased risk of non-metastatic cancer when the cfDNA fragment size distribution shows an increase in fragments having sizes of 50 bases to 105 bases in the subject compared to the healthy control. In some embodiments, the cfDNA fragment size distribution shows at least a 0.5% increase in fragments having sizes of 50 bases to 105 bases in the subject compared to the healthy control. In some embodiments, the cfDNA fragment size distribution shows at least a 1% increase in fragments having sizes of 50 bases to 105 bases in the subject compared to the healthy control. In some embodiments, the cfDNA fragment size distribution shows at least a 10% increase in fragments having sizes of 50 bases to 105 bases in the subject compared to the healthy control. In some embodiments, the subject does not have a diagnosis of metastatic cancer. In some embodiments, the subject has a tumor burden of less than 10%. In some embodiments, the cancer is selected from the group consisting of colon cancer, non-small cell lung cancer, small cell lung cancer, breast cancer, hepatocellular carcinoma, liver cancer, skin cancer, malignant melanoma, endometrial cancer, esophageal cancer, gastric cancer, ovarian cancer, pancreatic cancer, and brain cancer. In some embodiments, the method does not comprise isolating tumor cfDNA polynucleotides from total cfDNA polynucleotides. In some embodiments, the non-metastatic cancer is stage 0, stage 1, stage 2, or stage 3. In some embodiments, the method further comprises recommending administration of a chemotherapy to the subject. In some embodiments, the method further comprises recommending additional cancer monitoring to the subject. In some embodiments, the method further comprises enriching the plurality of cfDNA polynucleotides for one or more target sequences. In some embodiments, the plurality of sequencing reads are processed using a sequence(s) of the targeted primer(s) or capture probe(s).

Further provided herein are methods of detecting a tumor in a subject. In some embodiments, the method comprises: (a) obtaining a sample comprising a plurality of cell-free DNA (cfDNA) polynucleotides from a blood sample of the subject; (b) measuring a total cfDNA fragment size distribution for the plurality of cfDNA polynucleotides of the subject; (c) comparing the total cfDNA fragment size distribution for the plurality of cfDNA polynucleotides of the subject to the total cfDNA fragment size distribution for the plurality of cfDNA polynucleotides from a healthy control; and (d) detecting a tumor DNA when the cfDNA fragment size distribution shows an increase in fragments having sizes of up to 170 bases in the subject compared to the healthy control. In some embodiments, the method further comprises classifying the subject as having an increased risk of non-metastatic cancer when the cfDNA fragment size distribution shows an increase in fragments having sizes of up to 170 bases in the subject compared to the healthy control. In some embodiments, the method further comprises preparation of a single stranded DNA library from the cfDNA polynucleotides of the subject. In some embodiments, the method further comprises preparation of a double stranded DNA library from the cfDNA polynucleotides of the subject. In some embodiments, the method further comprises: (a) circularizing individual cfDNA polynucleotides of the plurality of cfDNA polynucleotides of the subject to form a plurality of circular polynucleotides; (b) amplifying the circular polynucleotides; (c) sequencing the amplified polynucleotides to produce a plurality of sequencing reads; and (d) determining a length for each individual cfDNA polynucleotide of the plurality of cfDNA polynucleotides of the subject. In some embodiments, the cfDNA fragment size distribution shows at least a 0.5% increase in fragments having sizes of up to 170 bases in the subject compared to the healthy control. In some embodiments, the cfDNA fragment size distribution shows at least a 1% increase in fragments having sizes of up to 170 bases in the subject compared to the healthy control. In some embodiments, the cfDNA fragment size distribution shows at least a 10% increase in fragments having sizes of up to 170 bases in the subject compared to the healthy control. In some embodiments, the method comprises classifying the subject as having an increased risk of non-metastatic cancer when the cfDNA fragment size distribution shows an increase in fragments having sizes of 50 bases to 125 bases in the subject compared to the healthy control. In some embodiments, the cfDNA fragment size distribution shows at least a 0.5% increase in fragments having sizes of 50 bases to 125 bases in the subject compared to the healthy control. In some embodiments, the cfDNA fragment size distribution shows at least a 1% increase in fragments having sizes of 50 bases to 125 bases in the subject compared to the healthy control. In some embodiments, the cfDNA fragment size distribution shows at least a 10% increase in fragments having sizes of 50 bases to 125 bases in the subject compared to the healthy control. In some embodiments, the method comprises classifying the subject as having an increased risk of non-metastatic cancer when the cfDNA fragment size distribution shows an increase in fragments having sizes of 50 bases to 115 bases in the subject compared to the healthy control. In some embodiments, the cfDNA fragment size distribution shows at least a 0.5% increase in fragments having sizes of 50 bases to 115 bases in the subject compared to the healthy control. In some embodiments, the cfDNA fragment size distribution shows at least a 1% increase in fragments having sizes of 50 bases to 115 bases in the subject compared to the healthy control. In some embodiments, the cfDNA fragment size distribution shows at least a 10% increase in fragments having sizes of 50 bases to 115 bases in the subject compared to the healthy control. In some embodiments, the method comprises classifying the subject as having an increased risk of non-metastatic cancer when the cfDNA fragment size distribution shows an increase in fragments having sizes of 50 bases to 105 bases in the subject compared to the healthy control. In some embodiments, the cfDNA fragment size distribution shows at least a 0.5% increase in fragments having sizes of 50 bases to 105 bases in the subject compared to the healthy control. In some embodiments, the cfDNA fragment size distribution shows at least a 1% increase in fragments having sizes of 50 bases to 105 bases in the subject compared to the healthy control. In some embodiments, the cfDNA fragment size distribution shows at least a 10% increase in fragments having sizes of 50 bases to 105 bases in the subject compared to the healthy control. In some embodiments, the subject does not have a diagnosis of metastatic cancer. In some embodiments, the subject has a tumor burden of less than 10%. In some embodiments, the cancer is selected from the group consisting of colon cancer, non-small cell lung cancer, small cell lung cancer, breast cancer, hepatocellular carcinoma, liver cancer, skin cancer, malignant melanoma, endometrial cancer, esophageal cancer, gastric cancer, ovarian cancer, pancreatic cancer, and brain cancer. In some embodiments, the method does not comprise isolating tumor cfDNA polynucleotides from total cfDNA polynucleotides. In some embodiments, the non-metastatic cancer is stage 0, stage 1, stage 2, or stage 3. In some embodiments, the method further comprises recommending administration of a chemotherapy to the subject. In some embodiments, the method further comprises recommending additional cancer monitoring to the subject. In some embodiments, the method further comprises enriching the plurality of cfDNA polynucleotides for one or more target sequences. In some embodiments, the plurality of sequencing reads are processed using a sequence(s) of the targeted primer(s) or capture probe(s).

Also provided herein are systems for detecting a non-metastatic cancer in a subject. In some embodiments, the system comprises (a) a computer configured to receive a user request to perform a detection of non-metastatic cancer on a sample; (b) an amplification system that performs a nucleic acid amplification reaction on cfDNA polynucleotides in the sample or a portion thereof in response to the user request; (c) a sequencing system that (i) sequences the amplified cfDNA polynucleotides to produce a plurality of sequencing reads; (ii) determines a length for each individual polynucleotide of the plurality of cfDNA polynucleotides of the subject; and (iii) produces a total cfDNA fragment size distribution for a plurality of cfDNA polynucleotides in the sample; and (d) a report generator that sends a report to a recipient, wherein the report contains results indicating a risk of non-metastatic cancer in the subject. In some embodiments, the system comprises an isolation system for isolating cell-free DNA (cfDNA) polynucleotides from a blood sample of the subject. In some embodiments, the system compares the total cfDNA fragment size distribution for the plurality of cfDNA polynucleotides of the subject to the total cfDNA fragment size distribution for the plurality of cfDNA polynucleotides from a healthy control. In some embodiments, the system detects a non-metastatic cancer in the subject when the cfDNA fragment size distribution shows an increase in fragments having sizes of up to 170 bases in the subject compared to the healthy control. In some embodiments, the report generator classifies the subject as having an increased risk of non-metastatic cancer when the cfDNA fragment size distribution shows an increase in fragments having sizes of up to 170 bases in the subject compared to the healthy control. In some embodiments, the system further comprises a preparation system for preparation of a single stranded DNA library from the cfDNA polynucleotides of the subject. In some embodiments, the system further comprises a preparation system for preparation of a double stranded DNA library from the cfDNA polynucleotides of the subject. In some embodiments, the amplification reaction comprises: circularizing individual cfDNA polynucleotides of the plurality of cfDNA polynucleotides of the subject to form a plurality of circular polynucleotides; and amplifying the circular polynucleotides. In some embodiments, the cfDNA fragment size distribution shows at least a 0.5% increase in fragments having sizes of up to 170 bases in the subject compared to the healthy control. In some embodiments, the cfDNA fragment size distribution shows at least a 1% increase in fragments having sizes of up to 170 bases in the subject compared to the healthy control. In some embodiments, the cfDNA fragment size distribution shows at least a 10% increase in fragments having sizes of up to 170 bases in the subject compared to the healthy control. In some embodiments, the report generator classifies the subject as having an increased risk of non-metastatic cancer when the cfDNA fragment size distribution shows an increase in fragments having sizes of 50 bases to 125 bases in the subject compared to the healthy control. In some embodiments, the cfDNA fragment size distribution shows at least a 0.5% increase in fragments having sizes of 50 bases to 125 bases in the subject compared to the healthy control. In some embodiments, the cfDNA fragment size distribution shows at least a 1% increase in fragments having sizes of 50 bases to 125 bases in the subject compared to the healthy control. In some embodiments, the cfDNA fragment size distribution shows at least a 10% increase in fragments having sizes of 50 bases to 125 bases in the subject compared to the healthy control. In some embodiments, the report generator classifies the subject as having an increased risk of non-metastatic cancer when the cfDNA fragment size distribution shows an increase in fragments having sizes of 50 bases to 115 bases in the subject compared to the healthy control. In some embodiments, the cfDNA fragment size distribution shows at least a 0.5% increase in fragments having sizes of 50 bases to 115 bases in the subject compared to the healthy control. In some embodiments, the cfDNA fragment size distribution shows at least a 1% increase in fragments having sizes of 50 bases to 115 bases in the subject compared to the healthy control. In some embodiments, the cfDNA fragment size distribution shows at least a 10% increase in fragments having sizes of 50 bases to 115 bases in the subject compared to the healthy control. In some embodiments, the report generator classifies the subject as having an increased risk of non-metastatic cancer when the cfDNA fragment size distribution shows an increase in fragments having sizes of 50 bases to 105 bases in the subject compared to the healthy control. In some embodiments, the cfDNA fragment size distribution shows at least a 0.5% increase in fragments having sizes of 50 bases to 105 bases in the subject compared to the healthy control. In some embodiments, the cfDNA fragment size distribution shows at least a 1% increase in fragments having sizes of 50 bases to 105 bases in the subject compared to the healthy control. In some embodiments, the cfDNA fragment size distribution shows at least a 10% increase in fragments having sizes of 50 bases to 105 bases in the subject compared to the healthy control. In some embodiments, the subject does not have a diagnosis of metastatic cancer. In some embodiments, the subject has a tumor burden of less than 10%. In some embodiments, the cancer is selected from the group consisting of colon cancer, non-small cell lung cancer, small cell lung cancer, breast cancer, hepatocellular carcinoma, liver cancer, skin cancer, malignant melanoma, endometrial cancer, esophageal cancer, gastric cancer, ovarian cancer, pancreatic cancer, and brain cancer. In some embodiments, the system does not isolate tumor cfDNA polynucleotides from total cfDNA polynucleotides. In some embodiments, the non-metastatic cancer is stage 0, stage 1, stage 2, or stage 3. In some embodiments, the report further contains a recommendation for administration of a chemotherapy to the subject. In some embodiments, the report further contains a recommendation of additional cancer monitoring to the subject. In some embodiments, the amplification system enriches the cfDNA polynucleotides for one or more target sequences.

Further provided herein are computer-readable mediums comprising codes that, upon execution by one or more processors, implement a method of detecting a non-metastatic cancer in a subject. In some embodiments, methods comprise: (a) receiving a customer request to perform a detection of non-metastatic cancer on a sample from a subject; (b) performing a nucleic acid amplification reaction on cfDNA polynucleotides in the sample or a portion thereof; (c) performing a sequencing analysis comprising the steps of (i) sequencing the amplified cfDNA polynucleotides to produce a plurality of sequencing reads; (ii) determining a length for each individual polynucleotide of the plurality of cfDNA polynucleotides of the subject; and (iii) producing a total cfDNA fragment size distribution for a plurality of cfDNA polynucleotides in the sample; and (d) generating a report that contains results indicating a risk of non-metastatic cancer in the subject. In some embodiments, the method comprises isolating cell-free DNA (cfDNA) polynucleotides from a blood sample of the subject. In some embodiments, the method comprises comparing the total cfDNA fragment size distribution for the plurality of cfDNA polynucleotides of the subject to the total cfDNA fragment size distribution for the plurality of cfDNA polynucleotides from a healthy control. In some embodiments, the method comprises detecting a non-metastatic cancer in the subject when the cfDNA fragment size distribution shows an increase in fragments having sizes of up to 170 bases in the subject compared to the healthy control. In some embodiments, the method comprises classifying the subject as having an increased risk of non-metastatic cancer when the cfDNA fragment size distribution shows an increase in fragments having sizes of up to 170 bases in the subject compared to the healthy control. In some embodiments, the method further comprises preparation of a single stranded DNA library from the cfDNA polynucleotides of the subject. In some embodiments, the method further comprises preparation of a double stranded DNA library from the cfDNA polynucleotides of the subject. In some embodiments, the amplification reaction comprises: circularizing individual cfDNA polynucleotides of the plurality of cfDNA polynucleotides of the subject to form a plurality of circular polynucleotides; and amplifying the circular polynucleotides. In some embodiments, the cfDNA fragment size distribution shows at least a 0.5% increase in fragments having sizes of up to 170 bases in the subject compared to the healthy control. In some embodiments, the cfDNA fragment size distribution shows at least a 1% increase in fragments having sizes of up to 170 bases in the subject compared to the healthy control. In some embodiments, the cfDNA fragment size distribution shows at least a 10% increase in fragments having sizes of up to 170 bases in the subject compared to the healthy control. In some embodiments, the method comprises classifying the subject as having an increased risk of non-metastatic cancer when the cfDNA fragment size distribution shows an increase in fragments having sizes of 50 bases to 125 bases in the subject compared to the healthy control. In some embodiments, the cfDNA fragment size distribution shows at least a 0.5% increase in fragments having sizes of 50 bases to 125 bases in the subject compared to the healthy control. In some embodiments, the cfDNA fragment size distribution shows at least a 1% increase in fragments having sizes of 50 bases to 125 bases in the subject compared to the healthy control. In some embodiments, the cfDNA fragment size distribution shows at least a 10% increase in fragments having sizes of 50 bases to 125 bases in the subject compared to the healthy control. In some embodiments, the method comprises the subject as having an increased risk of non-metastatic cancer when the cfDNA fragment size distribution shows an increase in fragments having sizes of 50 bases to 115 bases in the subject compared to the healthy control. In some embodiments, the cfDNA fragment size distribution shows at least a 0.5% increase in fragments having sizes of 50 bases to 115 bases in the subject compared to the healthy control. In some embodiments, the cfDNA fragment size distribution shows at least a 1% increase in fragments having sizes of 50 bases to 115 bases in the subject compared to the healthy control. In some embodiments, the cfDNA fragment size distribution shows at least a 10% increase in fragments having sizes of 50 bases to 115 bases in the subject compared to the healthy control. In some embodiments, the method comprises classifying the subject as having an increased risk of non-metastatic cancer when the cfDNA fragment size distribution shows an increase in fragments having sizes of 50 bases to 105 bases in the subject compared to the healthy control. In some embodiments, the cfDNA fragment size distribution shows at least a 0.5% increase in fragments having sizes of 50 bases to 105 bases in the subject compared to the healthy control. In some embodiments, the cfDNA fragment size distribution shows at least a 1% increase in fragments having sizes of 50 bases to 105 bases in the subject compared to the healthy control. In some embodiments, the cfDNA fragment size distribution shows at least a 10% increase in fragments having sizes of 50 bases to 105 bases in the subject compared to the healthy control. In some embodiments, the subject does not have a diagnosis of metastatic cancer. In some embodiments, the subject has a tumor burden of less than 10%. In some embodiments, the cancer is selected from the group consisting of colon cancer, non-small cell lung cancer, small cell lung cancer, breast cancer, hepatocellular carcinoma, liver cancer, skin cancer, malignant melanoma, endometrial cancer, esophageal cancer, gastric cancer, ovarian cancer, pancreatic cancer, and brain cancer. In some embodiments, the method does not comprise isolating tumor cfDNA polynucleotides from total cfDNA polynucleotides. In some embodiments, the non-metastatic cancer is stage 0, stage 1, stage 2, or stage 3. In some embodiments, the report further contains a recommendation for administration of a chemotherapy to the subject. In some embodiments, the method further comprises recommending additional cancer monitoring to the subject. In some embodiments, performing an amplification reaction comprises enriching the cfDNA polynucleotides for one or more target sequences. In some embodiments, the cfDNA polynucleotides are enriched for the one or more target sequences prior to or subsequent to performing the amplification reaction.

Further provided herein are methods of detecting a non-metastatic cancer in a subject, comprising: (a) obtaining a sample comprising a plurality of cell-free DNA (cfDNA) nucleic acid molecules of the subject; (b) measuring a total cfDNA fragment size distribution for the plurality of cfDNA nucleic acid molecules; and (c) determining that the subject has or is at increased risk of having a non-metastatic cancer when the total cfDNA fragment size distribution shows an increase in fragments as compared to a total cfDNA fragment size distribution for the plurality of nucleic acid molecules from a healthy control.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

An understanding of the features and advantages of the present invention will be obtained by reference to the following detailed description that sets forth illustrative embodiments, in which the principles of the invention are utilized, and the accompanying drawings of which:

FIG. 1A shows a size distribution of cell-free deoxyribonucleic acid (cfDNA) obtained from healthy individuals compared to individuals having cancer with mutant allele frequency of 0.

FIG. 1B shows a size distribution of cfDNA obtained from healthy individuals compared to individuals having cancer with mutant allele frequency greater than 0 and less than 5.

FIG. 1C shows a size distribution of cfDNA obtained from healthy individuals compared to individuals having cancer with mutant allele frequency of greater or equal to 5 and less than 20.

FIG. 1D shows a size distribution of cfDNA obtained from healthy individuals compared to individuals having cancer with mutant allele frequency of greater than or equal to 20.

FIG. 2A shows a graph illustrating size difference observed between Colorectal Cancer (CRC) samples and healthy samples.

FIG. 2B shows overall mutant allele frequency versus mutant allele frequency (MAF) calculated using molecules in size range 50 bases-150 bases. MAF in small size range is higher than over MAF, indicating enrichment of ctDNA in small fragments. However, for most samples with overall MAF<1%, the MAF in small size range are still lower than 5%. Dashed line: y=x.

FIG. 3 illustrates an example computer system.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used herein, the term “about” or “approximately” means within an acceptable error range for the particular value as determined by one of ordinary skill in the art, which may depend in part on how the value is measured or determined, i.e., the limitations of the measurement system. For example, “about” can mean within 1 or more than 1 standard deviation, per the practice in the art. As another example, “about” can mean a range of up to 20%, up to 10%, up to 5%, or up to 1% of a given value. With respect to biological systems or processes, the term “about” can mean within an order of magnitude, such as within 5-fold or within 2-fold of a value. Where particular values are described in the application and claims, unless otherwise stated, the term “about” means within an acceptable error range for the particular value.

As used herein, the terms “polynucleotide”, “nucleotide”, “nucleotide sequence”, “nucleic acid” and “oligonucleotide” generally refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: cell-free nucleic acids, cell-free DNA (cfDNA), circulating tumor DNA (ctDNA), coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

As used herein, the terms “total cell-free nucleic acids” and “total cell-free polynucleotides”, which include, but are not limited to “total cell-free deoxyribonucleic acid (DNA)”, “total cell-free DNA”, “total cfDNA”, “total cell-free ribonucleic acid (RNA)”, “total cell-free RNA”, and “total cfRNA” generally refer to a population of cell-free nucleic acids that has not been enriched for cancer associated mutations. Total cell-free nucleic acids, including total cell-free DNA or total cell-free RNA, may be the entire population of cell-free nucleic acids in an individual. Total cell-free nucleic acids, including total cell-free DNA or total cell-free RNA, may also be a representative subset of the population of cell-free nucleic acids in an individual that has not been enriched for cancer associated mutations.

The term “subject” as used herein, generally refers to an individual, such as a vertebrate. A vertebrate may be a mammal (e.g., a human). Mammals include, but are not limited to, murines, simians, humans, farm animals, sport animals, and pets. Tissues, cells, and their progeny of a biological entity obtained in vivo or cultured in vitro are also encompassed. The subject may be a patient. The subject may be symptomatic with respect to a disease (e.g., cancer). As an alternative, the subject may be asymptomatic with respect to the disease.

The terms “early stage cancer” and “non-metastatic cancer” used interchangeably herein refer to a cancer that has not yet metastasized in an individual (i.e., the cancer has not left its initial location to spread to other locations). The exact staging depends upon the type of cancer, details for which are provided herein.

The terms “tumor burden” and “tumor load,” as used herein, generally refer to a size of a tumor or an amount of a disease (e.g., cancer) in a body of a subject.

The term “healthy control,” as used herein, generally refers to a point of reference for a healthy state of a subject(s). The healthy control may be from a subject(s) not having or suspected of having a disease (e.g., cancer), or from a subject(s) having or suspected of having the disease but from a location that may otherwise be used as a non-disease reference (e.g., white blood cells). The healthy control may be a genome or a portion of a genome. The healthy control may be ribonucleic acid molecule(s) and/or deoxyribonucleic acid molecule(s).

The term “sample,” as used herein, generally refers to a sample derived from or obtained from a subject, such as a mammal (e.g., a human). The sample may be a biological sample. Samples may include, but are not limited to, hair, finger nails, skin, sweat, tears, ocular fluids, nasal swab or nasopharyngeal wash, sputum, throat swab, saliva, mucus, blood, serum, plasma, placental fluid, amniotic fluid, cord blood, emphatic fluids, cavity fluids, earwax, oil, glandular secretions, bile, lymph, pus, microbiota, meconium, breast milk, bone marrow, bone, CNS tissue, cerebrospinal fluid, adipose tissue, synovial fluid, stool, gastric fluid, urine, semen, vaginal secretions, stomach, small intestine, large intestine, rectum, pancreas, liver, kidney, bladder, lung, and other tissues and fluids derived from or obtained from a subject. The biological sample may be a cell-free (or cell free) biological sample.

The term “cell-free,” as used herein, generally refers to a sample derived from or obtained from a subject that is free from cells. Cell-free biological samples may include, but are not limited to, blood, serum, plasma, nasal swab or nasopharyngeal wash, saliva, urine, gastric fluid, tears, stool, mucus, sweat, earwax, oil, glandular secretion, bile, lymph, cerebrospinal fluid, tissue, semen, vaginal fluid, interstitial fluids, including interstitial fluids derived from tumor tissue, ocular fluids, spinal fluid, throat swab, breath, hair, finger nails, skin, biopsy, placental fluid, amniotic fluid, cord blood, emphatic fluids, cavity fluids, sputum, pus, microbiota, meconium, breast milk and/or other excretions.

Provided herein are methods, systems, and compositions for detecting or diagnosing non-metastatic or early stage cancer in a subject. Methods provided herein also comprise measuring a cell-free DNA (cfDNA) size distribution in a sample from a subject and detecting a tumor cfDNA in a sample from a subject. Methods herein are based on the discovery that the size distribution of cfDNA in subjects having cancer shows an increase in cfDNA having a reduced size compared with a cfDNA size distribution in healthy subjects. This discovery illustrates that it is possible to detect cancer at an early stage by observing the amount of cfDNA in the smaller size range, for example cfDNA having sizes of up to 170 bases.

### Detection of Non-Metastatic Cancer

Provided herein are methods of detecting a non-metastatic cancer in a subject. In some embodiments, some such methods comprise obtaining a sample comprising a plurality of cell-free deoxyribonucleic acid (cfDNA) polynucleotides of the subject and measuring a total cfDNA fragment size distribution for the plurality of cfDNA polynucleotides of the subject. Next, the total cfDNA fragment size distribution may be compared for the plurality of cfDNA polynucleotides of the subject to the total cfDNA fragment size distribution for the plurality of cfDNA polynucleotides from a healthy control. The subject sample may then be used to determine that the subject has or is at risk of having non-metastatic cancer when the cfDNA fragment size distribution shows an increase in fragments having sizes of 10 bases to 170 bases in the subject compared to the healthy control. In some embodiments, the method further comprises enriching the plurality of cfDNA polynucleotides for one or more target sequences. In some cases, the method does not comprise enriching for a target sequence. In some cases, the method does not comprise aligning or mapping a cfDNA polynucleotide sequence to a reference genome.

Also provided herein are methods of detecting a non-metastatic cancer in a subject. In some embodiments, some such methods comprise obtaining a sample comprising a plurality of cfDNA polynucleotides of the subject and measuring a total cfDNA fragment size distribution for the plurality of cfDNA polynucleotides of the subject by sequencing. Next, the total cfDNA fragment size distribution may be compared for the plurality of cfDNA polynucleotides of the subject to the total cfDNA fragment size distribution for the plurality of cfDNA polynucleotides from a healthy control. The subject sample may then be used to determine that the subject has or is at risk of having non-metastatic cancer when the cfDNA fragment size distribution shows an increase in fragments having sizes of 10 bases to 170 bases in the subject compared to the healthy control. In some embodiments, the method further comprises enriching the plurality of cfDNA polynucleotides for one or more target sequences. In some cases, the method does not comprise enriching for a target sequence. In some cases, the method does not comprise aligning or mapping a cfDNA polynucleotide sequence to a reference genome.

Further provided herein are methods of detecting a non-metastatic cancer in a subject comprising obtaining a sample comprising a plurality of cfDNA polynucleotides of the subject and measuring a total cfDNA fragment size distribution for the plurality of cfDNA polynucleotides of the subject. Next, the total cfDNA fragment size distribution for the plurality of cfDNA polynucleotides of the subject may be compared to the total cfDNA fragment size distribution for the plurality of cfDNA polynucleotides from a healthy control. The subject sample may then be used to determine that the subject has or is at risk of having an increased risk (e.g., increased by at least 1%, 10%, 20%, 30%, 40%, 50%, or more as compared to a healthy subject or population of healthy subjects) of non-metastatic cancer when the cfDNA fragment size distribution shows an increase in the subject compared to the healthy control in at least 2, 3, 4, 5, 6, 7, 8, 9, 10, or more cfDNA fragments having sizes in a range of 10 bases to 170 bases.

In some cases, methods of detecting non-metastatic cancer provided herein comprise preparation of a deoxyribonucleic acid (DNA) library from polynucleotides in the sample of the subject. In some cases, the method further comprises preparation of a single stranded DNA library from the cfDNA polynucleotides of the subject. Any suitable method of preparing a single stranded DNA library may be used in methods herein. For example, the method of preparing a single stranded DNA library may comprise denaturing the DNA sample to create a plurality of ssDNA; ligating an adapter to the 3′ end of the ssDNA molecules; synthesizing a second strand using a primer complementary to the adapter; ligating a double stranded adapter to the extension products; amplifying the second strand using primers targeting the first and second adapters (for example, using PCR); and sequencing the library on a sequencer. An additional method of single stranded library preparation may comprise denaturing the DNA sample to create a plurality of ssDNA; ligating an adapter to the 3′ end of the ssDNA molecules; synthesizing the second strand by using a primer complementary to the adapter; ligating a double stranded adapter to the extension products; amplifying the second strand (for example, by PCR) the second strand using primers targeting the first and second adapters; in some cases enriching for the regions of interest using hybridization with capture probes; amplifying (for example, by PCR) the captured products; and sequencing the library on a sequencer. The method may further comprise analyzing the resulting sequences (e.g., sequencing reads) using a sequence(s) of the primer(s) or capture probe(s), such as, for example, by grouping the resulting sequences (e.g., sequencing reads) according to the sequences of the primer(s) or capture probe(s)—for example, individual sequencing reads are distributed into individual groups each corresponding to a sequence of a primer or capture probe. This may be performed without aligning such resulting sequences with a genome.

The method may further comprise preparation of a double stranded DNA library from the cfDNA polynucleotides of the subject. Any suitable method of preparing a double stranded DNA may be used in methods herein. For example, the method of preparing a double stranded DNA library may comprise ligating sequencing adapters to the 5′ and 3′ ends of a plurality of DNA fragments and sequencing the library on a sequencer. An additional method of double stranded DNA library preparation may comprise ligating adapters to the 5′ and 3′ ends of a plurality of DNA fragments; attaching the full adapter sequences to the ligated fragments through PCR using primers that are complementary to the ligated adapters; and sequencing the library on a sequencer. A further method may comprise ligating adapters to the 5′ and 3′ ends of a plurality of DNA fragments; amplifying the ligated product through PCR that are complementary to the ligated adapters; in some cases enriching for the regions of interest through hybridization with capture probes; PCR amplifying the captured products; and sequencing the library on a sequencer. An additional method of double stranded library preparation may comprise ligating adapters to the 5′ and 3′ ends of a plurality of DNA fragments; amplifying the ligated product through PCR using primers that are complementary to the ligated adapters; circularizing the double stranded PCR products or denature and circularize the single stranded PCR products; in some cases, enriching for the regions of interest by PCR using primers targeting specific genes; and sequencing the library on a sequencer. The method may further comprise analyzing the resulting sequences (e.g., sequencing reads) using a sequence(s) of the primer(s) or capture probe(s), such as, for example, by grouping the resulting sequences (e.g., sequencing reads) according to the sequences of the primer(s) or capture probe(s)—for example, individual sequencing reads are distributed into individual groups each corresponding to a sequence of a primer or capture probe. This may be performed without aligning such resulting sequences with a genome.

Methods of measuring a cfDNA size distribution provided herein may comprise any suitable method of measuring DNA size. Examples of suitable methods of measuring a cfDNA size distribution include but are not limited to sequencing, bioanalyzer fragment analysis, PCR, qPCR, high throughput gel electrophoresis, high throughput capillary electrophoresis, and any other suitable method that provides sizes of DNA fragments.

Methods of detecting non-metastatic cancer provided herein may further comprise and amplification and sequencing steps including one or more of (a) circularizing individual cfDNA polynucleotides of the plurality of cfDNA polynucleotides of the subject to form a plurality of circular polynucleotides; (b) amplifying the circular polynucleotides; (c) sequencing the amplified polynucleotides to produce a plurality of sequencing reads; and (d) determining a length for each individual cfDNA polynucleotide of the plurality of cfDNA polynucleotides of the subject. Methods of detecting early stage or non-metastatic cancer, in some embodiments, do not comprise isolating tumor cfDNA polynucleotides from total cfDNA polynucleotides.

In methods of detecting non-metastatic cancer provided herein, the cfDNA fragment size distribution may show at least an increase in fragments having sizes of 10 bases to 170 bases in the subject compared to the healthy control. In some embodiments, the cfDNA fragment size distribution shows at least an increase in fragments having sizes of 10 bases to 50 bases, 10 bases to 70 bases, 10 bases to 105 bases, 10 bases to 115 bases, 10 bases to 125 bases, 10 bases to 170 bases, 25 bases to 50 bases, 25 bases to 70 bases, 25 bases to 105 bases, 25 bases to 115 bases, 25 bases to 125 bases, 25 bases to 170 bases, 50 bases to 70 bases, 50 bases to 105 bases, 50 bases to 115 bases, 50 bases to 125 bases, 50 bases to 170 bases, 70 bases to 105 bases, 70 bases to 115 bases, 70 bases to 125 bases, 70 bases to 170 bases, 105 bases to 115 bases, 105 bases to 125 bases, 105 bases to 170 bases, 115 bases to 125 bases, 115 bases to 170 bases, or 125 bases to 170 bases in the subject compared to the healthy control.

In methods of detecting non-metastatic cancer provided herein, the cfDNA fragment size distribution may show at least an increase in fragments having sizes of 10 bases to 170 bases in the subject compared to the healthy control. In some embodiments, the cfDNA fragment size distribution shows at least a 0.25% increase in fragments having sizes of 10 bases to 170 bases in the subject compared to the healthy control. In some embodiments, the cfDNA fragment size distribution shows at least a 0.5% increase in fragments having sizes of 10 bases to 170 bases in the subject compared to the healthy control. In some embodiments, the cfDNA fragment size distribution shows at least a 1% increase in fragments having sizes of 10 bases to 170 bases in the subject compared to the healthy control. In some embodiments, the cfDNA fragment size distribution shows at least a 2% increase in fragments having sizes of 10 bases to 170 bases in the subject compared to the healthy control. In some embodiments, the cfDNA fragment size distribution shows at least a 3% increase in fragments having sizes of 10 bases to 170 bases in the subject compared to the healthy control. In some embodiments, the cfDNA fragment size distribution shows at least a 4% increase in fragments having sizes of 10 bases to 170 bases in the subject compared to the healthy control. In some embodiments, the cfDNA fragment size distribution shows at least a 5% increase in fragments having sizes of 10 bases to 170 bases in the subject compared to the healthy control. In some embodiments, the cfDNA fragment size distribution shows at least a 6% increase in fragments having sizes of 10 bases to 170 bases in the subject compared to the healthy control. In some embodiments, the cfDNA fragment size distribution shows at least a 7% increase in fragments having sizes of 10 bases to 170 bases in the subject compared to the healthy control. In some embodiments, the cfDNA fragment size distribution shows at least a 8% increase in fragments having sizes of 10 bases to 170 bases in the subject compared to the healthy control. In some embodiments, the cfDNA fragment size distribution shows at least a 9% increase in fragments having sizes of 10 bases to 170 bases in the subject compared to the healthy control. In some embodiments, the cfDNA fragment size distribution shows at least a 10% increase in fragments having sizes of 10 bases to 170 bases in the subject compared to the healthy control. In some embodiments, the cfDNA fragment size distribution shows at least a 15% increase in fragments having sizes of 10 bases to 170 bases in the subject compared to the healthy control. In some embodiments, the cfDNA fragment size distribution shows at least a 20% increase in fragments having sizes of 10 bases to 170 bases in the subject compared to the healthy control.

In methods of detecting non-metastatic cancer provided herein, the cfDNA fragment size distribution may show at least an increase in fragments having sizes of 50 bases to 170 bases in the subject compared to the healthy control. In some embodiments, the cfDNA fragment size distribution shows at least a 0.25% increase in fragments having sizes of 50 bases to 170 bases in the subject compared to the healthy control. In some embodiments, the cfDNA fragment size distribution shows at least a 0.5% increase in fragments having sizes of 50 bases to 170 bases in the subject compared to the healthy control. In some embodiments, the cfDNA fragment size distribution shows at least a 1% increase in fragments having sizes of 50 bases to 170 bases in the subject compared to the healthy control. In some embodiments, the cfDNA fragment size distribution shows at least a 2% increase in fragments having sizes of 50 bases to 170 bases in the subject compared to the healthy control. In some embodiments, the cfDNA fragment size distribution shows at least a 3% increase in fragments having sizes of 50 bases to 170 bases in the subject compared to the healthy control. In some embodiments, the cfDNA fragment size distribution shows at least a 4% increase in fragments having sizes of 50 bases to 170 bases in the subject compared to the healthy control. In some embodiments, the cfDNA fragment size distribution shows at least a 5% increase in fragments having sizes of 50 bases to 170 bases in the subject compared to the healthy control. In some embodiments, the cfDNA fragment size distribution shows at least a 6% increase in fragments having sizes of 50 bases to 170 bases in the subject compared to the healthy control. In some embodiments, the cfDNA fragment size distribution shows at least a 7% increase in fragments having sizes of 50 bases to 170 bases in the subject compared to the healthy control. In some embodiments, the cfDNA fragment size distribution shows at least a 8% increase in fragments having sizes of 50 bases to 170 bases in the subject compared to the healthy control. In some embodiments, the cfDNA fragment size distribution shows at least a 9% increase in fragments having sizes of 50 bases to 170 bases in the subject compared to the healthy control. In some embodiments, the cfDNA fragment size distribution shows at least a 10% increase in fragments having sizes of 50 bases to 170 bases in the subject compared to the healthy control. In some embodiments, the cfDNA fragment size distribution shows at least a 15% increase in fragments having sizes of 50 bases to 170 bases in the subject compared to the healthy control. In some embodiments, the cfDNA fragment size distribution shows at least a 20% increase in fragments having sizes of 50 bases to 170 bases in the subject compared to the healthy control. Such increase may be at least a 1%, 5%, 10%, 20%, 30%, 40% 50% or more increase in fragments having sizes of 50 bases to 170 bases in the subject compared to the healthy control.

In certain methods of detecting non-metastatic cancer provided herein, the method may comprise classifying the subject as having an increased risk of non-metastatic cancer when the cfDNA fragment size distribution shows an increase in fragments having sizes of 50 bases to 125 bases in the subject compared to the healthy control. In some embodiments, the cfDNA fragment size distribution shows at least a 0.25% increase in fragments having sizes of 50 bases to 125 bases in the subject compared to the healthy control. In some embodiments, the cfDNA fragment size distribution shows at least a 0.5% increase in fragments having sizes of 50 bases to 125 bases in the subject compared to the healthy control. In some embodiments, the cfDNA fragment size distribution shows at least a 1% increase in fragments having sizes of 50 bases to 125 bases in the subject compared to the healthy control. In some embodiments, the cfDNA fragment size distribution shows at least a 2% increase in fragments having sizes of 50 bases to 125 bases in the subject compared to the healthy control. In some embodiments, the cfDNA fragment size distribution shows at least a 3% increase in fragments having sizes of 50 bases to 125 bases in the subject compared to the healthy control. In some embodiments, the cfDNA fragment size distribution shows at least a 4% increase in fragments having sizes of 50 bases to 125 bases in the subject compared to the healthy control. In some embodiments, the cfDNA fragment size distribution shows at least a 5% increase in fragments having sizes of 50 bases to 125 bases in the subject compared to the healthy control. In some embodiments, the cfDNA fragment size distribution shows at least a 6% increase in fragments having sizes of 50 bases to 125 bases in the subject compared to the healthy control. In some embodiments, the cfDNA fragment size distribution shows at least a 7% increase in fragments having sizes of 50 bases to 125 bases in the subject compared to the healthy control. In some embodiments, the cfDNA fragment size distribution shows at least a 8% increase in fragments having sizes of 50 bases to 125 bases in the subject compared to the healthy control. In some embodiments, the cfDNA fragment size distribution shows at least a 9% increase in fragments having sizes of 50 bases to 125 bases in the subject compared to the healthy control. In some embodiments, the cfDNA fragment size distribution shows at least a 10% increase in fragments having sizes of 50 bases to 125 bases in the subject compared to the healthy control. In some embodiments, the cfDNA fragment size distribution shows at least a 15% increase in fragments having sizes of 50 bases to 125 bases in the subject compared to the healthy control. In some embodiments, the cfDNA fragment size distribution shows at least a 20% increase in fragments having sizes of 50 bases to 125 bases in the subject compared to the healthy control.

In certain methods of detecting non-metastatic cancer provided herein, the method may comprise classifying the subject as having an increased risk of non-metastatic cancer when the cfDNA fragment size distribution shows an increase in fragments having sizes of 50 bases to 115 bases in the subject compared to the healthy control. In some embodiments, the cfDNA fragment size distribution shows at least a 0.25% increase in fragments having sizes of 50 bases to 115 bases in the subject compared to the healthy control. In some embodiments, the cfDNA fragment size distribution shows at least a 0.5% increase in fragments having sizes of 50 bases to 115 bases in the subject compared to the healthy control. In some embodiments, the cfDNA fragment size distribution shows at least a 1% increase in fragments having sizes of 50 bases to 115 bases in the subject compared to the healthy control. In some embodiments, the cfDNA fragment size distribution shows at least a 2% increase in fragments having sizes of 50 bases to 115 bases in the subject compared to the healthy control. In some embodiments, the cfDNA fragment size distribution shows at least a 3% increase in fragments having sizes of 50 bases to 115 bases in the subject compared to the healthy control. In some embodiments, the cfDNA fragment size distribution shows at least a 4% increase in fragments having sizes of 50 bases to 115 bases in the subject compared to the healthy control. In some embodiments, the cfDNA fragment size distribution shows at least a 5% increase in fragments having sizes of 50 bases to 115 bases in the subject compared to the healthy control. In some embodiments, the cfDNA fragment size distribution shows at least a 6% increase in fragments having sizes of 50 bases to 115 bases in the subject compared to the healthy control. In some embodiments, the cfDNA fragment size distribution shows at least a 7% increase in fragments having sizes of 50 bases to 115 bases in the subject compared to the healthy control. In some embodiments, the cfDNA fragment size distribution shows at least a 8% increase in fragments having sizes of 50 bases to 115 bases in the subject compared to the healthy control. In some embodiments, the cfDNA fragment size distribution shows at least a 9% increase in fragments having sizes of 50 bases to 115 bases in the subject compared to the healthy control. In some embodiments, the cfDNA fragment size distribution shows at least a 10% increase in fragments having sizes of 50 bases to 115 bases in the subject compared to the healthy control. In some embodiments, the cfDNA fragment size distribution shows at least a 15% increase in fragments having sizes of 50 bases to 115 bases in the subject compared to the healthy control. In some embodiments, the cfDNA fragment size distribution shows at least a 20% increase in fragments having sizes of 50 bases to 115 bases in the subject compared to the healthy control.

In certain methods of detecting non-metastatic cancer provided herein, the method may comprise classifying the subject as having an increased risk of non-metastatic cancer when the cfDNA fragment size distribution shows an increase in fragments having sizes of 50 bases to 105 bases in the subject compared to the healthy control. In some embodiments, the cfDNA fragment size distribution shows at least a 0.25% increase in fragments having sizes of 50 bases to 105 bases in the subject compared to the healthy control. In some embodiments, the cfDNA fragment size distribution shows at least a 0.5% increase in fragments having sizes of 50 bases to 105 bases in the subject compared to the healthy control. In some embodiments, the cfDNA fragment size distribution shows at least a 1% increase in fragments having sizes of 50 bases to 105 bases in the subject compared to the healthy control. In some embodiments, the cfDNA fragment size distribution shows at least a 2% increase in fragments having sizes of 50 bases to 105 bases in the subject compared to the healthy control. In some embodiments, the cfDNA fragment size distribution shows at least a 3% increase in fragments having sizes of 50 bases to 105 bases in the subject compared to the healthy control. In some embodiments, the cfDNA fragment size distribution shows at least a 4% increase in fragments having sizes of 50 bases to 105 bases in the subject compared to the healthy control. In some embodiments, the cfDNA fragment size distribution shows at least a 5% increase in fragments having sizes of 50 bases to 105 bases in the subject compared to the healthy control. In some embodiments, the cfDNA fragment size distribution shows at least a 6% increase in fragments having sizes of 50 bases to 105 bases in the subject compared to the healthy control. In some embodiments, the cfDNA fragment size distribution shows at least a 7% increase in fragments having sizes of 50 bases to 105 bases in the subject compared to the healthy control. In some embodiments, the cfDNA fragment size distribution shows at least a 8% increase in fragments having sizes of 50 bases to 105 bases in the subject compared to the healthy control. In some embodiments, the cfDNA fragment size distribution shows at least a 9% increase in fragments having sizes of 50 bases to 105 bases in the subject compared to the healthy control. In some embodiments, the cfDNA fragment size distribution shows at least a 10% increase in fragments having sizes of 50 bases to 105 bases in the subject compared to the healthy control. In some embodiments, the cfDNA fragment size distribution shows at least a 15% increase in fragments having sizes of 50 bases to 105 bases in the subject compared to the healthy control. In some embodiments, the cfDNA fragment size distribution shows at least a 20% increase in fragments having sizes of 50 bases to 105 bases in the subject compared to the healthy control.

In methods of detecting early stage or non-metastatic cancer provided herein, in certain cases, the plurality of cfDNA polynucleotides comprises only a small portion of circulating tumor DNA (ctDNA). For example, in some embodiments, the plurality of cfDNA polynucleotides comprises less than 25% ctDNA. In some embodiments, the plurality of cfDNA polynucleotides comprises less than 20% ctDNA. In some embodiments, the plurality of cfDNA polynucleotides comprises less than 15% ctDNA. In some embodiments, the plurality of cfDNA polynucleotides comprises less than 10% ctDNA. In some embodiments, the plurality of cfDNA polynucleotides comprises less than 5% ctDNA.

In certain aspects of methods of detecting early stage or non-metastatic cancer provided herein, the subject has cancer but in the very early stages, in some instances, before it can be detected using conventional methods. In some cases, the subject does not have a diagnosis of metastatic cancer. Alternatively, the subject has a low tumor burden, for example, in some cases, the subject has a tumor burden of less than 20%. In some embodiments, the subject has a tumor burden of less than 10%. In some embodiments, the subject has a tumor burden of less than 9%. In some embodiments, the subject has a tumor burden of less than 8%. In some embodiments, the subject has a tumor burden of less than 7%. In some embodiments, the subject has a tumor burden of less than 6%. In some embodiments, the subject has a tumor burden of less than 5%. Alternatively, the early stage cancers detected using methods herein comprise a non-metastatic cancer having an early stage, which is sometimes classified using a numerical classification system. In some cases, an early stage cancer is staged according to the type of cancer. In some embodiments, the non-metastatic cancer is stage 0, stage 1, stage 2, or stage 3.

Cancers detectable using methods herein may include, but are not limited to, colon cancer, non-small cell lung cancer, small cell lung cancer, breast cancer, hepatocellular carcinoma, liver cancer, skin cancer, malignant melanoma, endometrial cancer, esophageal cancer, gastric cancer, ovarian cancer, pancreatic cancer, or brain cancer.

In methods of detecting early stage or non-metastatic cancer provided herein, in some cases, the method comprises additional steps for addressing the cancer. In some embodiments, the method comprises recommending a treatment for the cancer in the subject. In some embodiments, the method comprises recommending administration of a chemotherapy to the subject. In some embodiments, the method comprises recommending additional cancer monitoring to the subject.

Measuring a cfDNA Size Distribution

Further provided herein are methods of measuring a cell-free deoxyribonucleic acid (cfDNA) size distribution in a blood sample from a subject. In some embodiments, methods comprise obtaining a sample comprising a plurality of cfDNA polynucleotides from the blood sample of the subject and measuring a total cfDNA fragment size distribution for the plurality of cfDNA polynucleotides of the subject. In further embodiments, methods comprise comparing the total cfDNA fragment size distribution for the plurality of cfDNA polynucleotides of the subject to the total cfDNA fragment size distribution for the plurality of cfDNA polynucleotides from a healthy control and classifying the subject as having an increased risk of non-metastatic cancer when the cfDNA fragment size distribution shows an increase in fragments having sizes of 50 bases to 170 bases in the subject compared to the healthy control. In some embodiments, the method further comprises enriching the plurality of cfDNA polynucleotides for one or more target sequences. In some cases, the method does not comprise enriching for a target sequence. In some cases, the method does not comprise aligning or mapping a cfDNA polynucleotide sequence to a reference genome.

Methods of measuring a cfDNA size distribution provided herein may comprise any suitable method of measuring DNA size. Suitable methods of measuring a cfDNA size distribution include, but are not limited to, sequencing, bioanalyzer fragment analysis, PCR, qPCR, high throughput gel electrophoresis, high throughput capillary electrophoresis, or any other suitable method that provides sizes of DNA fragments.

In methods of measuring a cfDNA size distribution provided herein, in certain cases, the plurality of cfDNA polynucleotides comprises only a small portion of circulating tumor DNA (ctDNA). For example, in some embodiments, the plurality of cfDNA polynucleotides comprises less than 25% ctDNA. In some embodiments, the plurality of cfDNA polynucleotides comprises less than 20% ctDNA. In some embodiments, the plurality of cfDNA polynucleotides comprises less than 15% ctDNA. In some embodiments, the plurality of cfDNA polynucleotides comprises less than 10% ctDNA. In some embodiments, the plurality of cfDNA polynucleotides comprises less than 5% ctDNA.

In some cases, methods of measuring a cfDNA size distribution provided herein comprise preparation of a DNA library from polynucleotides in the sample of the subject. For example, in some embodiments, the method further comprises preparation of a single stranded DNA library from the cfDNA polynucleotides of the subject. Any suitable method of preparing a single stranded DNA library may be used in methods herein. For example, the method of preparing a single stranded DNA library comprises denaturing the DNA sample to create a plurality of ssDNA; ligating an adapter to the 3′ end of the ssDNA molecules; synthesizing a second strand using a primer complementary to the adapter; ligating a double stranded adapter to the extension products; amplifying the second strand using primers targeting the first and second adapters (for example, using PCR); and sequencing the library on a sequencer. An additional method of single stranded library preparation may comprise denaturing the DNA sample to create a plurality of ssDNA; ligating an adapter to the 3′ end of the ssDNA molecules; synthesizing the second strand by using a primer complementary to the adapter; ligating a double stranded adapter to the extension products; amplifying the second strand (for example, by PCR) the second strand using primers targeting the first and second adapters; in some cases enriching for the regions of interest using hybridization with capture probes; amplifying (for example, by PCR) the captured products; and sequencing the library on a sequencer. The method may further comprise analyzing the resulting sequences (e.g., sequencing reads) using a sequence(s) of the primer(s) or capture probe(s), such as, for example, by grouping the resulting sequences (e.g., sequencing reads) according to the sequences of the primer(s) or capture probe(s)—for example, individual sequencing reads are distributed into individual groups each corresponding to a sequence of a primer or capture probe. This may be performed without aligning such resulting sequences with a genome.

Alternatively or in combination, the method may further comprise preparation of a double stranded DNA library from the cfDNA polynucleotides of the subject. Any suitable method of preparing a double stranded DNA library may be used in methods herein. For example, the method of preparing a double stranded DNA library may comprise ligating sequencing adapters to the 5′ and 3′ ends of a plurality of DNA fragments and sequencing the library on a sequencer. An additional method of double stranded DNA library preparation may comprise ligating adapters to the 5′ and 3′ ends of a plurality of DNA fragments; attaching the full adapter sequences to the ligated fragments through PCR using primers that are complementary to the ligated adapters; and sequencing the library on a sequencer. A further method may comprise ligating adapters to the 5′ and 3′ ends of a plurality of DNA fragments; amplifying the ligated product through PCR that are complementary to the ligated adapters; in some cases enriching for the regions of interest through hybridization with capture probes; PCR amplifying the captured products; and sequencing the library on a sequencer. An additional method of double stranded library preparation may comprise ligating adapters to the 5′ and 3′ ends of a plurality of DNA fragments; amplifying the ligated product through PCR using primers that are complementary to the ligated adapters; circularizing the double stranded PCR products or denature and circularize the single stranded PCR products; in some cases enriching for the regions of interest by PCR using primers targeting specific genes; and sequencing the library on a sequencer. The method may further comprise analyzing the resulting sequences (e.g., sequencing reads) using a sequence(s) of the primer(s) or capture probe(s), such as, for example, by grouping the resulting sequences (e.g., sequencing reads) according to the sequences of the primer(s) or capture probe(s)—for example, individual sequencing reads are distributed into individual groups each corresponding to a sequence of a primer or capture probe. This may be performed without aligning such resulting sequences with a genome.

Methods of measuring a cfDNA size distribution provided herein may further comprise and amplification and sequencing steps including one or more of (a) circularizing individual cfDNA polynucleotides of the plurality of cfDNA polynucleotides of the subject to form a plurality of circular polynucleotides; (b) amplifying the circular polynucleotides; (c) sequencing the amplified polynucleotides to produce a plurality of sequencing reads; and (d) determining a length for each individual cfDNA polynucleotide of the plurality of cfDNA polynucleotides of the subject. Methods of measuring a cfDNA size distribution, in some embodiments, do not comprise isolating tumor cfDNA polynucleotides from total cfDNA polynucleotides.

In methods of measuring a cfDNA size distribution provided herein, the cfDNA fragment size distribution may show at least an increase in fragments having sizes of 10 bases to 170 bases in the subject compared to the healthy control. In some embodiments, the cfDNA fragment size distribution shows at least an increase in fragments having sizes of 10 bases to 50 bases, 10 bases to 70 bases, 10 bases to 105 bases, 10 bases to 115 bases, 10 bases to 125 bases, 10 bases to 170 bases, 25 bases to 50 bases, 25 bases to 70 bases, 25 bases to 105 bases, 25 bases to 115 bases, 25 bases to 125 bases, 25 bases to 170 bases, 50 bases to 70 bases, 50 bases to 105 bases, 50 bases to 115 bases, 50 bases to 125 bases, 50 bases to 170 bases, 70 bases to 105 bases, 70 bases to 115 bases, 70 bases to 125 bases, 70 bases to 170 bases, 105 bases to 115 bases, 105 bases to 125 bases, 105 bases to 170 bases, 115 bases to 125 bases, 115 bases to 170 bases, or 125 bases to 170 bases in the subject compared to the healthy control.

In methods of measuring a cfDNA size distribution provided herein, the cfDNA fragment size distribution may show at least an increase in fragments having sizes of 10 bases to 170 bases in the subject compared to the healthy control. In some embodiments, the cfDNA fragment size distribution shows at least a 0.25% increase in fragments having sizes of 10 bases to 170 bases in the subject compared to the healthy control. In some embodiments, the cfDNA fragment size distribution shows at least a 0.5% increase in fragments having sizes of 10 bases to 170 bases in the subject compared to the healthy control. In some embodiments, the cfDNA fragment size distribution shows at least a 1% increase in fragments having sizes of 10 bases to 170 bases in the subject compared to the healthy control. In some embodiments, the cfDNA fragment size distribution shows at least a 2% increase in fragments having sizes of 10 bases to 170 bases in the subject compared to the healthy control. In some embodiments, the cfDNA fragment size distribution shows at least a 3% increase in fragments having sizes of 10 bases to 170 bases in the subject compared to the healthy control. In some embodiments, the cfDNA fragment size distribution shows at least a 4% increase in fragments having sizes of 10 bases to 170 bases in the subject compared to the healthy control. In some embodiments, the cfDNA fragment size distribution shows at least a 5% increase in fragments having sizes of 10 bases to 170 bases in the subject compared to the healthy control. In some embodiments, the cfDNA fragment size distribution shows at least a 6% increase in fragments having sizes of 10 bases to 170 bases in the subject compared to the healthy control. In some embodiments, the cfDNA fragment size distribution shows at least a 7% increase in fragments having sizes of 10 bases to 170 bases in the subject compared to the healthy control. In some embodiments, the cfDNA fragment size distribution shows at least a 8% increase in fragments having sizes of 10 bases to 170 bases in the subject compared to the healthy control. In some embodiments, the cfDNA fragment size distribution shows at least a 9% increase in fragments having sizes of 10 bases to 170 bases in the subject compared to the healthy control. In some embodiments, the cfDNA fragment size distribution shows at least a 10% increase in fragments having sizes of 10 bases to 170 bases in the subject compared to the healthy control. In some embodiments, the cfDNA fragment size distribution shows at least a 15% increase in fragments having sizes of 10 bases to 170 bases in the subject compared to the healthy control. In some embodiments, the cfDNA fragment size distribution shows at least a 20% increase in fragments having sizes of 10 bases to 170 bases in the subject compared to the healthy control.

In methods of measuring a cfDNA size distribution provided herein, the cfDNA fragment size distribution may show at least an increase in fragments having sizes of 50 bases to 170 bases in the subject compared to the healthy control. In some embodiments, the cfDNA fragment size distribution shows at least a 0.25% increase in fragments having sizes of 50 bases to 170 bases in the subject compared to the healthy control. In some embodiments, the cfDNA fragment size distribution shows at least a 0.5% increase in fragments having sizes of 50 bases to 170 bases in the subject compared to the healthy control. In some embodiments, the cfDNA fragment size distribution shows at least a 1% increase in fragments having sizes of 50 bases to 170 bases in the subject compared to the healthy control. In some embodiments, the cfDNA fragment size distribution shows at least a 2% increase in fragments having sizes of 50 bases to 170 bases in the subject compared to the healthy control. In some embodiments, the cfDNA fragment size distribution shows at least a 3% increase in fragments having sizes of 50 bases to 170 bases in the subject compared to the healthy control. In some embodiments, the cfDNA fragment size distribution shows at least a 4% increase in fragments having sizes of 50 bases to 170 bases in the subject compared to the healthy control. In some embodiments, the cfDNA fragment size distribution shows at least a 5% increase in fragments having sizes of 50 bases to 170 bases in the subject compared to the healthy control. In some embodiments, the cfDNA fragment size distribution shows at least a 6% increase in fragments having sizes of 50 bases to 170 bases in the subject compared to the healthy control. In some embodiments, the cfDNA fragment size distribution shows at least a 7% increase in fragments having sizes of 50 bases to 170 bases in the subject compared to the healthy control. In some embodiments, the cfDNA fragment size distribution shows at least a 8% increase in fragments having sizes of 50 bases to 170 bases in the subject compared to the healthy control. In some embodiments, the cfDNA fragment size distribution shows at least a 9% increase in fragments having sizes of 50 bases to 170 bases in the subject compared to the healthy control. In some embodiments, the cfDNA fragment size distribution shows at least a 10% increase in fragments having sizes of 50 bases to 170 bases in the subject compared to the healthy control. In some embodiments, the cfDNA fragment size distribution shows at least a 15% increase in fragments having sizes of 50 bases to 170 bases in the subject compared to the healthy control. In some embodiments, the cfDNA fragment size distribution shows at least a 20% increase in fragments having sizes of 50 bases to 170 bases in the subject compared to the healthy control.

Further provided herein are methods of measuring a cfDNA size distribution in a subject comprising obtaining a sample comprising a plurality of cfDNA polynucleotides of the subject and measuring a total cfDNA fragment size distribution for the plurality of cfDNA polynucleotides of the subject. Next, the total cfDNA fragment size distribution for the plurality of cfDNA polynucleotides of the subject may be compared to the total cfDNA fragment size distribution for the plurality of cfDNA polynucleotides from a healthy control. The subject sample may then be used to determine that the subject has or is at risk of having non-metastatic cancer when the cfDNA fragment size distribution shows an increase in the subject compared to the healthy control in at least 2, 3, 4, 5, 6, 7, 8, 9, 10, or more cfDNA fragments having sizes in a range of 10 bases to 170 bases.

In certain methods of measuring a cfDNA size distribution provided herein, the method may comprise classifying the subject as having an increased risk of non-metastatic cancer when the cfDNA fragment size distribution shows an increase in fragments having sizes of 50 bases to 125 bases in the subject compared to the healthy control. In some embodiments, the cfDNA fragment size distribution shows at least a 0.25% increase in fragments having sizes of 50 bases to 125 bases in the subject compared to the healthy control. In some embodiments, the cfDNA fragment size distribution shows at least a 0.5% increase in fragments having sizes of 50 bases to 125 bases in the subject compared to the healthy control. In some embodiments, the cfDNA fragment size distribution shows at least a 1% increase in fragments having sizes of 50 bases to 125 bases in the subject compared to the healthy control. In some embodiments, the cfDNA fragment size distribution shows at least a 2% increase in fragments having sizes of 50 bases to 125 bases in the subject compared to the healthy control. In some embodiments, the cfDNA fragment size distribution shows at least a 3% increase in fragments having sizes of 50 bases to 125 bases in the subject compared to the healthy control. In some embodiments, the cfDNA fragment size distribution shows at least a 4% increase in fragments having sizes of 50 bases to 125 bases in the subject compared to the healthy control. In some embodiments, the cfDNA fragment size distribution shows at least a 5% increase in fragments having sizes of 50 bases to 125 bases in the subject compared to the healthy control. In some embodiments, the cfDNA fragment size distribution shows at least a 6% increase in fragments having sizes of 50 bases to 125 bases in the subject compared to the healthy control. In some embodiments, the cfDNA fragment size distribution shows at least a 7% increase in fragments having sizes of 50 bases to 125 bases in the subject compared to the healthy control. In some embodiments, the cfDNA fragment size distribution shows at least a 8% increase in fragments having sizes of 50 bases to 125 bases in the subject compared to the healthy control. In some embodiments, the cfDNA fragment size distribution shows at least a 9% increase in fragments having sizes of 50 bases to 125 bases in the subject compared to the healthy control. In some embodiments, the cfDNA fragment size distribution shows at least a 10% increase in fragments having sizes of 50 bases to 125 bases in the subject compared to the healthy control. In some embodiments, the cfDNA fragment size distribution shows at least a 15% increase in fragments having sizes of 50 bases to 125 bases in the subject compared to the healthy control. In some embodiments, the cfDNA fragment size distribution shows at least a 20% increase in fragments having sizes of 50 bases to 125 bases in the subject compared to the healthy control.

In certain methods of measuring a cfDNA size distribution provided herein, the method may comprise classifying the subject as having an increased risk of non-metastatic cancer when the cfDNA fragment size distribution shows an increase in fragments having sizes of 50 bases to 115 bases in the subject compared to the healthy control. In some embodiments, the cfDNA fragment size distribution shows at least a 0.25% increase in fragments having sizes of 50 bases to 115 bases in the subject compared to the healthy control. In some embodiments, the cfDNA fragment size distribution shows at least a 0.5% increase in fragments having sizes of 50 bases to 115 bases in the subject compared to the healthy control. In some embodiments, the cfDNA fragment size distribution shows at least a 1% increase in fragments having sizes of 50 bases to 115 bases in the subject compared to the healthy control. In some embodiments, the cfDNA fragment size distribution shows at least a 2% increase in fragments having sizes of 50 bases to 115 bases in the subject compared to the healthy control. In some embodiments, the cfDNA fragment size distribution shows at least a 3% increase in fragments having sizes of 50 bases to 115 bases in the subject compared to the healthy control. In some embodiments, the cfDNA fragment size distribution shows at least a 4% increase in fragments having sizes of 50 bases to 115 bases in the subject compared to the healthy control. In some embodiments, the cfDNA fragment size distribution shows at least a 5% increase in fragments having sizes of 50 bases to 115 bases in the subject compared to the healthy control. In some embodiments, the cfDNA fragment size distribution shows at least a 6% increase in fragments having sizes of 50 bases to 115 bases in the subject compared to the healthy control. In some embodiments, the cfDNA fragment size distribution shows at least a 7% increase in fragments having sizes of 50 bases to 115 bases in the subject compared to the healthy control. In some embodiments, the cfDNA fragment size distribution shows at least a 8% increase in fragments having sizes of 50 bases to 115 bases in the subject compared to the healthy control. In some embodiments, the cfDNA fragment size distribution shows at least a 9% increase in fragments having sizes of 50 bases to 115 bases in the subject compared to the healthy control. In some embodiments, the cfDNA fragment size distribution shows at least a 10% increase in fragments having sizes of 50 bases to 115 bases in the subject compared to the healthy control. In some embodiments, the cfDNA fragment size distribution shows at least a 15% increase in fragments having sizes of 50 bases to 115 bases in the subject compared to the healthy control. In some embodiments, the cfDNA fragment size distribution shows at least a 20% increase in fragments having sizes of 50 bases to 115 bases in the subject compared to the healthy control.

In certain methods of measuring a cfDNA size distribution provided herein, the method comprises classifying the subject as having an increased risk of non-metastatic cancer when the cfDNA fragment size distribution shows an increase in fragments having sizes of 50 bases to 105 bases in the subject compared to the healthy control. In some embodiments, the cfDNA fragment size distribution shows at least a 0.25% increase in fragments having sizes of 50 bases to 105 bases in the subject compared to the healthy control. In some embodiments, the cfDNA fragment size distribution shows at least a 0.5% increase in fragments having sizes of 50 bases to 105 bases in the subject compared to the healthy control. In some embodiments, the cfDNA fragment size distribution shows at least a 1% increase in fragments having sizes of 50 bases to 105 bases in the subject compared to the healthy control. In some embodiments, the cfDNA fragment size distribution shows at least a 2% increase in fragments having sizes of 50 bases to 105 bases in the subject compared to the healthy control. In some embodiments, the cfDNA fragment size distribution shows at least a 3% increase in fragments having sizes of 50 bases to 105 bases in the subject compared to the healthy control. In some embodiments, the cfDNA fragment size distribution shows at least a 4% increase in fragments having sizes of 50 bases to 105 bases in the subject compared to the healthy control. In some embodiments, the cfDNA fragment size distribution shows at least a 5% increase in fragments having sizes of 50 bases to 105 bases in the subject compared to the healthy control. In some embodiments, the cfDNA fragment size distribution shows at least a 6% increase in fragments having sizes of 50 bases to 105 bases in the subject compared to the healthy control. In some embodiments, the cfDNA fragment size distribution shows at least a 7% increase in fragments having sizes of 50 bases to 105 bases in the subject compared to the healthy control. In some embodiments, the cfDNA fragment size distribution shows at least a 8% increase in fragments having sizes of 50 bases to 105 bases in the subject compared to the healthy control. In some embodiments, the cfDNA fragment size distribution shows at least a 9% increase in fragments having sizes of 50 bases to 105 bases in the subject compared to the healthy control. In some embodiments, the cfDNA fragment size distribution shows at least a 10% increase in fragments having sizes of 50 bases to 105 bases in the subject compared to the healthy control. In some embodiments, the cfDNA fragment size distribution shows at least a 15% increase in fragments having sizes of 50 bases to 105 bases in the subject compared to the healthy control. In some embodiments, the cfDNA fragment size distribution shows at least a 20% increase in fragments having sizes of 50 bases to 105 bases in the subject compared to the healthy control.

In certain aspects of methods of measuring a cfDNA size distribution provided herein, the subject may have cancer but in the very early stages, in some instances, before it can be detected using conventional methods. In some cases, the subject does not have a diagnosis of metastatic cancer. In some cases, the subject has a low tumor burden, for example, in some cases, the subject has a tumor burden of less than 20%. In some embodiments, the subject has a tumor burden of less than 10%. In some embodiments, the subject has a tumor burden of less than 9%. In some embodiments, the subject has a tumor burden of less than 8%. In some embodiments, the subject has a tumor burden of less than 7%. In some embodiments, the subject has a tumor burden of less than 6%. In some embodiments, the subject has a tumor burden of less than 5%. In some cases, the early stage cancers detected using methods herein comprise a non-metastatic cancer having an early stage, which may be classified using a numerical classification system. In some cases, an early stage cancer is staged according to the type of cancer. In some embodiments, the non-metastatic cancer is stage 0, stage 1, stage 2, or stage 3.

Cancers detectable using methods herein may include, but are not limited to, colon cancer, non-small cell lung cancer, small cell lung cancer, breast cancer, hepatocellular carcinoma, liver cancer, skin cancer, malignant melanoma, endometrial cancer, esophageal cancer, gastric cancer, ovarian cancer, pancreatic cancer, or brain cancer.

In methods of measuring a cfDNA size distribution provided herein, in some cases, the method comprises additional steps for addressing the cancer. In some embodiments, the method comprises recommending a treatment for the cancer in the subject. In some embodiments, the method comprises recommending administration of a chemotherapy to the subject. In some embodiments, the method comprises recommending additional cancer monitoring to the subject.

### Tumor Detection

Additionally provided herein are methods of detecting a tumor in a blood sample from a subject. In some embodiments, some such methods comprise obtaining a sample comprising a plurality of cell-free deoxyribonucleic acid (cfDNA) polynucleotides from the blood sample of the subject and measuring a total cfDNA fragment size distribution for the plurality of cfDNA polynucleotides of the subject. Next, the total cfDNA fragment size distribution for the plurality of cfDNA polynucleotides of the subject may be compared to the total cfDNA fragment size distribution for the plurality of cfDNA polynucleotides from a healthy control. The subject sample may then be used to detecting a tumor DNA when the cfDNA fragment size distribution shows an increase in fragments having sizes of 50 bases to 170 bases in the subject compared to the healthy control. In some embodiments, the method further comprises enriching the plurality of cfDNA polynucleotides for one or more target sequences. In some cases, the method does not comprise enriching for a target sequence. In some cases, the method does not comprise aligning or mapping a cfDNA polynucleotide sequence to a reference genome.

In certain methods of detecting a tumor cfDNA provided herein, the method may further comprise classifying the subject as having an increased risk of non-metastatic cancer when the cfDNA fragment size distribution shows an increase in fragments having sizes of 50 bases to 170 bases in the subject compared to the healthy control.

In methods of detecting a tumor cfDNA provided herein, in certain cases, the plurality of cfDNA polynucleotides comprises only a small portion of circulating tumor DNA (ctDNA). For example, in some embodiments, the plurality of cfDNA polynucleotides comprises less than 25% ctDNA. In some embodiments, the plurality of cfDNA polynucleotides comprises less than 20% ctDNA. In some embodiments, the plurality of cfDNA polynucleotides comprises less than 15% ctDNA. In some embodiments, the plurality of cfDNA polynucleotides comprises less than 10% ctDNA. In some embodiments, the plurality of cfDNA polynucleotides comprises less than 5% ctDNA.

In some cases, methods of detecting a tumor cfDNA provided herein comprise preparation of a DNA library from polynucleotides in the sample of the subject. For example, in some embodiments, the method further comprises preparation of a single stranded DNA library from the cfDNA polynucleotides of the subject. Any suitable method of preparing a single stranded DNA library may be used in methods herein. For example, the method of preparing a single stranded DNA library may comprise denaturing the DNA sample to create a plurality of ssDNA; ligating an adapter to the 3′ end of the ssDNA molecules; synthesizing a second strand using a primer complementary to the adapter; ligating a double stranded adapter to the extension products; amplifying the second strand using primers targeting the first and second adapters (for example, using PCR); and sequencing the library on a sequencer. An additional method of single stranded library preparation may comprises denaturing the DNA sample to create a plurality of ssDNA; ligating an adapter to the 3′ end of the ssDNA molecules; synthesizing the second strand by using a primer complementary to the adapter; ligating a double stranded adapter to the extension products; amplifying the second strand (for example, by PCR) the second strand using primers targeting the first and second adapters; in some cases enriching for the regions of interest using hybridization with capture probes; amplifying (for example, by PCR) the captured products; and sequencing the library on a sequencer. The method may further comprise analyzing the resulting sequences (e.g., sequencing reads) using a sequence(s) of the primer(s) or capture probe(s), such as, for example, by grouping the resulting sequences (e.g., sequencing reads) according to the sequences of the primer(s) or capture probe(s)—for example, individual sequencing reads are distributed into individual groups each corresponding to a sequence of a primer or capture probe. This may be performed without aligning such resulting sequences with a genome.

The method may further comprise preparation of a double stranded DNA library from the cfDNA polynucleotides of the subject. Any suitable method of preparing a double stranded DNA library may be used in methods herein. For example, the method of preparing a double stranded DNA library may comprise ligating sequencing adapters to the 5′ and 3′ ends of a plurality of DNA fragments and sequencing the library on a sequencer. An additional method of double stranded DNA library preparation may comprise ligating adapters to the 5′ and 3′ ends of a plurality of DNA fragments; attaching the full adapter sequences to the ligated fragments through PCR using primers that are complementary to the ligated adapters; and sequencing the library on a sequencer. A further method may comprise ligating adapters to the 5′ and 3′ ends of a plurality of DNA fragments; amplifying the ligated product through PCR that are complementary to the ligated adapters; in some cases enriching for the regions of interest through hybridization with capture probes; PCR amplifying the captured products; and sequencing the library on a sequencer. An additional method of double stranded library preparation may comprise ligating adapters to the 5′ and 3′ ends of a plurality of DNA fragments; amplifying the ligated product through PCR using primers that are complementary to the ligated adapters; circularizing the double stranded PCR products or denature and circularize the single stranded PCR products; in some cases enriching for the regions of interest by PCR using primers targeting specific genes; and sequencing the library on a sequencer. The method may further comprise analyzing the resulting sequences (e.g., sequencing reads) using a sequence(s) of the primer(s) or capture probe(s), such as, for example, by grouping the resulting sequences (e.g., sequencing reads) according to the sequences of the primer(s) or capture probe(s)—for example, individual sequencing reads are distributed into individual groups each corresponding to a sequence of a primer or capture probe. This may be performed without aligning such resulting sequences with a genome.

Methods of measuring a cfDNA size distribution provided herein may comprise any suitable method of measuring DNA size. Suitable methods of measuring a cfDNA size distribution include, but are not limited to, sequencing, bioanalyzer fragment analysis, PCR, qPCR, high throughput gel electrophoresis, high throughput capillary electrophoresis, or any other suitable method that provides sizes of DNA fragments.

Methods of detecting a tumor provided herein may further comprise and amplification and sequencing steps including one or more of (a) circularizing individual cfDNA polynucleotides of the plurality of cfDNA polynucleotides of the subject to form a plurality of circular polynucleotides; (b) amplifying the circular polynucleotides; (c) sequencing the amplified polynucleotides to produce a plurality of sequencing reads; and (d) determining a length for each individual cfDNA polynucleotide of the plurality of cfDNA polynucleotides of the subject. Methods of detecting a tumor cfDNA, in some embodiments, do not comprise isolating tumor cfDNA polynucleotides from total cfDNA polynucleotides.

In certain methods of detecting a tumor provided herein, the method may further comprise classifying the subject as having an increased risk of non-metastatic cancer when the cfDNA fragment size distribution shows an increase in fragments having sizes of 10 bases to 170 bases in the subject compared to the healthy control. In some embodiments, the cfDNA fragment size distribution shows at least an increase in fragments having sizes of 10 bases to 50 bases, 10 bases to 70 bases, 10 bases to 105 bases, 10 bases to 115 bases, 10 bases to 125 bases, 10 bases to 170 bases, 25 bases to 50 bases, 25 bases to 70 bases, 25 bases to 105 bases, 25 bases to 115 bases, 25 bases to 125 bases, 25 bases to 170 bases, 50 bases to 70 bases, 50 bases to 105 bases, 50 bases to 115 bases, 50 bases to 125 bases, 50 bases to 170 bases, 70 bases to 105 bases, 70 bases to 115 bases, 70 bases to 125 bases, 70 bases to 170 bases, 105 bases to 115 bases, 105 bases to 125 bases, 105 bases to 170 bases, 115 bases to 125 bases, 115 bases to 170 bases, or 125 bases to 170 bases in the subject compared to the healthy control.

In certain methods of detecting a tumor provided herein, the method may further comprise classifying the subject as having an increased risk of non-metastatic cancer when the cfDNA fragment size distribution shows an increase in fragments having sizes of 10 bases to 170 bases in the subject compared to the healthy control. In some embodiments, the cfDNA fragment size distribution shows at least a 0.25% increase in fragments having sizes of 10 bases to 170 bases in the subject compared to the healthy control. In some embodiments, the cfDNA fragment size distribution shows at least a 0.5% increase in fragments having sizes of 10 bases to 170 bases in the subject compared to the healthy control. In some embodiments, the cfDNA fragment size distribution shows at least a 1% increase in fragments having sizes of 10 bases to 170 bases in the subject compared to the healthy control. In some embodiments, the cfDNA fragment size distribution shows at least a 2% increase in fragments having sizes of 10 bases to 170 bases in the subject compared to the healthy control. In some embodiments, the cfDNA fragment size distribution shows at least a 3% increase in fragments having sizes of 10 bases to 170 bases in the subject compared to the healthy control. In some embodiments, the cfDNA fragment size distribution shows at least a 4% increase in fragments having sizes of 10 bases to 170 bases in the subject compared to the healthy control. In some embodiments, the cfDNA fragment size distribution shows at least a 5% increase in fragments having sizes of 10 bases to 170 bases in the subject compared to the healthy control. In some embodiments, the cfDNA fragment size distribution shows at least a 6% increase in fragments having sizes of 10 bases to 170 bases in the subject compared to the healthy control. In some embodiments, the cfDNA fragment size distribution shows at least a 7% increase in fragments having sizes of 10 bases to 170 bases in the subject compared to the healthy control. In some embodiments, the cfDNA fragment size distribution shows at least an 8% increase in fragments having sizes of 10 bases to 170 bases in the subject compared to the healthy control. In some embodiments, the cfDNA fragment size distribution shows at least a 9% increase in fragments having sizes of 10 bases to 170 bases in the subject compared to the healthy control. In some embodiments, the cfDNA fragment size distribution shows at least a 10% increase in fragments having sizes of 10 bases to 170 bases in the subject compared to the healthy control. In some embodiments, the cfDNA fragment size distribution shows at least a 15% increase in fragments having sizes of 10 bases to 170 bases in the subject compared to the healthy control. In some embodiments, the cfDNA fragment size distribution shows at least a 20% increase in fragments having sizes of 10 bases to 170 bases in the subject compared to the healthy control.

In certain methods of detecting a tumor provided herein, the method may further comprise classifying the subject as having an increased risk of non-metastatic cancer when the cfDNA fragment size distribution shows an increase in fragments having sizes of 50 bases to 170 bases in the subject compared to the healthy control. In some embodiments, the cfDNA fragment size distribution shows at least a 0.25% increase in fragments having sizes of 50 bases to 170 bases in the subject compared to the healthy control. In some embodiments, the cfDNA fragment size distribution shows at least a 0.5% increase in fragments having sizes of 50 bases to 170 bases in the subject compared to the healthy control. In some embodiments, the cfDNA fragment size distribution shows at least a 1% increase in fragments having sizes of 50 bases to 170 bases in the subject compared to the healthy control. In some embodiments, the cfDNA fragment size distribution shows at least a 2% increase in fragments having sizes of 50 bases to 170 bases in the subject compared to the healthy control. In some embodiments, the cfDNA fragment size distribution shows at least a 3% increase in fragments having sizes of 50 bases to 170 bases in the subject compared to the healthy control. In some embodiments, the cfDNA fragment size distribution shows at least a 4% increase in fragments having sizes of 50 bases to 170 bases in the subject compared to the healthy control. In some embodiments, the cfDNA fragment size distribution shows at least a 5% increase in fragments having sizes of 50 bases to 170 bases in the subject compared to the healthy control. In some embodiments, the cfDNA fragment size distribution shows at least a 6% increase in fragments having sizes of 50 bases to 170 bases in the subject compared to the healthy control. In some embodiments, the cfDNA fragment size distribution shows at least a 7% increase in fragments having sizes of 50 bases to 170 bases in the subject compared to the healthy control. In some embodiments, the cfDNA fragment size distribution shows at least an 8% increase in fragments having sizes of 50 bases to 170 bases in the subject compared to the healthy control. In some embodiments, the cfDNA fragment size distribution shows at least a 9% increase in fragments having sizes of 50 bases to 170 bases in the subject compared to the healthy control. In some embodiments, the cfDNA fragment size distribution shows at least a 10% increase in fragments having sizes of 50 bases to 170 bases in the subject compared to the healthy control. In some embodiments, the cfDNA fragment size distribution shows at least a 15% increase in fragments having sizes of 50 bases to 170 bases in the subject compared to the healthy control. In some embodiments, the cfDNA fragment size distribution shows at least a 20% increase in fragments having sizes of 50 bases to 170 bases in the subject compared to the healthy control.

Further provided herein are methods of detecting a tumor in a subject comprising obtaining a sample comprising a plurality of cfDNA polynucleotides of the subject and measuring a total cfDNA fragment size distribution for the plurality of cfDNA polynucleotides of the subject. Next, the total cfDNA fragment size distribution for the plurality of cfDNA polynucleotides of the subject may be compared to the total cfDNA fragment size distribution for the plurality of cfDNA polynucleotides from a healthy control; and classifying the subject as having a tumor when the cfDNA fragment size distribution shows an increase in the subject compared to the healthy control in at least 2, 3, 4, 5, 6, 7, 8, 9, 10, or more cfDNA fragments having sizes in a range of 10 bases to 170 bases.

In certain methods of detecting a tumor provided herein, the method may comprise classifying the subject as having an increased risk of non-metastatic cancer when the cfDNA fragment size distribution shows an increase in fragments having sizes of 50 bases to 125 bases in the subject compared to the healthy control. In some embodiments, the cfDNA fragment size distribution shows at least a 0.25% increase in fragments having sizes of 50 bases to 125 bases in the subject compared to the healthy control. In some embodiments, the cfDNA fragment size distribution shows at least a 0.5% increase in fragments having sizes of 50 bases to 125 bases in the subject compared to the healthy control. In some embodiments, the cfDNA fragment size distribution shows at least a 1% increase in fragments having sizes of 50 bases to 125 bases in the subject compared to the healthy control. In some embodiments, the cfDNA fragment size distribution shows at least a 2% increase in fragments having sizes of 50 bases to 125 bases in the subject compared to the healthy control. In some embodiments, the cfDNA fragment size distribution shows at least a 3% increase in fragments having sizes of 50 bases to 125 bases in the subject compared to the healthy control. In some embodiments, the cfDNA fragment size distribution shows at least a 4% increase in fragments having sizes of 50 bases to 125 bases in the subject compared to the healthy control. In some embodiments, the cfDNA fragment size distribution shows at least a 5% increase in fragments having sizes of 50 bases to 125 bases in the subject compared to the healthy control. In some embodiments, the cfDNA fragment size distribution shows at least a 6% increase in fragments having sizes of 50 bases to 125 bases in the subject compared to the healthy control. In some embodiments, the cfDNA fragment size distribution shows at least a 7% increase in fragments having sizes of 50 bases to 125 bases in the subject compared to the healthy control. In some embodiments, the cfDNA fragment size distribution shows at least a 8% increase in fragments having sizes of 50 bases to 125 bases in the subject compared to the healthy control. In some embodiments, the cfDNA fragment size distribution shows at least a 9% increase in fragments having sizes of 50 bases to 125 bases in the subject compared to the healthy control. In some embodiments, the cfDNA fragment size distribution shows at least a 10% increase in fragments having sizes of 50 bases to 125 bases in the subject compared to the healthy control. In some embodiments, the cfDNA fragment size distribution shows at least a 15% increase in fragments having sizes of 50 bases to 125 bases in the subject compared to the healthy control. In some embodiments, the cfDNA fragment size distribution shows at least a 20% increase in fragments having sizes of 50 bases to 125 bases in the subject compared to the healthy control.

In certain methods of detecting a tumor provided herein, the method may comprise classifying the subject as having an increased risk of non-metastatic cancer when the cfDNA fragment size distribution shows an increase in fragments having sizes of 50 bases to 115 bases in the subject compared to the healthy control. In some embodiments, the cfDNA fragment size distribution shows at least a 0.25% increase in fragments having sizes of 50 bases to 115 bases in the subject compared to the healthy control. In some embodiments, the cfDNA fragment size distribution shows at least a 0.5% increase in fragments having sizes of 50 bases to 115 bases in the subject compared to the healthy control. In some embodiments, the cfDNA fragment size distribution shows at least a 1% increase in fragments having sizes of 50 bases to 115 bases in the subject compared to the healthy control. In some embodiments, the cfDNA fragment size distribution shows at least a 2% increase in fragments having sizes of 50 bases to 115 bases in the subject compared to the healthy control. In some embodiments, the cfDNA fragment size distribution shows at least a 3% increase in fragments having sizes of 50 bases to 115 bases in the subject compared to the healthy control. In some embodiments, the cfDNA fragment size distribution shows at least a 4% increase in fragments having sizes of 50 bases to 115 bases in the subject compared to the healthy control. In some embodiments, the cfDNA fragment size distribution shows at least a 5% increase in fragments having sizes of 50 bases to 115 bases in the subject compared to the healthy control. In some embodiments, the cfDNA fragment size distribution shows at least a 6% increase in fragments having sizes of 50 bases to 115 bases in the subject compared to the healthy control. In some embodiments, the cfDNA fragment size distribution shows at least a 7% increase in fragments having sizes of 50 bases to 115 bases in the subject compared to the healthy control. In some embodiments, the cfDNA fragment size distribution shows at least a 8% increase in fragments having sizes of 50 bases to 115 bases in the subject compared to the healthy control. In some embodiments, the cfDNA fragment size distribution shows at least a 9% increase in fragments having sizes of 50 bases to 115 bases in the subject compared to the healthy control. In some embodiments, the cfDNA fragment size distribution shows at least a 10% increase in fragments having sizes of 50 bases to 115 bases in the subject compared to the healthy control. In some embodiments, the cfDNA fragment size distribution shows at least a 15% increase in fragments having sizes of 50 bases to 115 bases in the subject compared to the healthy control. In some embodiments, the cfDNA fragment size distribution shows at least a 20% increase in fragments having sizes of 50 bases to 115 bases in the subject compared to the healthy control.

In certain methods of detecting a tumor provided herein, the method may comprise classifying the subject as having an increased risk of non-metastatic cancer when the cfDNA fragment size distribution shows an increase in fragments having sizes of 50 bases to 105 bases in the subject compared to the healthy control. In some embodiments, the cfDNA fragment size distribution shows at least a 0.25% increase in fragments having sizes of 50 bases to 105 bases in the subject compared to the healthy control. In some embodiments, the cfDNA fragment size distribution shows at least a 0.5% increase in fragments having sizes of 50 bases to 105 bases in the subject compared to the healthy control. In some embodiments, the cfDNA fragment size distribution shows at least a 1% increase in fragments having sizes of 50 bases to 105 bases in the subject compared to the healthy control. In some embodiments, the cfDNA fragment size distribution shows at least a 2% increase in fragments having sizes of 50 bases to 105 bases in the subject compared to the healthy control. In some embodiments, the cfDNA fragment size distribution shows at least a 3% increase in fragments having sizes of 50 bases to 105 bases in the subject compared to the healthy control. In some embodiments, the cfDNA fragment size distribution shows at least a 4% increase in fragments having sizes of 50 bases to 105 bases in the subject compared to the healthy control. In some embodiments, the cfDNA fragment size distribution shows at least a 5% increase in fragments having sizes of 50 bases to 105 bases in the subject compared to the healthy control. In some embodiments, the cfDNA fragment size distribution shows at least a 6% increase in fragments having sizes of 50 bases to 105 bases in the subject compared to the healthy control. In some embodiments, the cfDNA fragment size distribution shows at least a 7% increase in fragments having sizes of 50 bases to 105 bases in the subject compared to the healthy control. In some embodiments, the cfDNA fragment size distribution shows at least a 8% increase in fragments having sizes of 50 bases to 105 bases in the subject compared to the healthy control. In some embodiments, the cfDNA fragment size distribution shows at least a 9% increase in fragments having sizes of 50 bases to 105 bases in the subject compared to the healthy control. In some embodiments, the cfDNA fragment size distribution shows at least a 10% increase in fragments having sizes of 50 bases to 105 bases in the subject compared to the healthy control. In some embodiments, the cfDNA fragment size distribution shows at least a 15% increase in fragments having sizes of 50 bases to 105 bases in the subject compared to the healthy control. In some embodiments, the cfDNA fragment size distribution shows at least a 20% increase in fragments having sizes of 50 bases to 105 bases in the subject compared to the healthy control.

In certain aspects of methods of detecting a tumor cfDNA provided herein, the subject may have cancer but in the very early stages, in some instances, before it can be detected using conventional methods. In some cases, the subject does not have a diagnosis of metastatic cancer. Alternatively, the subject has a low tumor burden, for example, in some cases, the subject has a tumor burden of less than 20%. In some embodiments, the subject has a tumor burden of less than 10%. In some embodiments, the subject has a tumor burden of less than 9%. In some embodiments, the subject has a tumor burden of less than 8%. In some embodiments, the subject has a tumor burden of less than 7%. In some embodiments, the subject has a tumor burden of less than 6%. In some embodiments, the subject has a tumor burden of less than 5%. In some cases, the early stage cancers detected using methods herein comprise a non-metastatic cancer having an early stage, which is sometimes classified using a numerical classification system. In some cases, an early stage cancer is staged according to the type of cancer. In some embodiments, the non-metastatic cancer is stage 0, stage 1, stage 2, or stage 3.

Cancers and tumor cfDNA detectable using methods herein may include, but are not limited to, colon cancer, non-small cell lung cancer, small cell lung cancer, breast cancer, hepatocellular carcinoma, liver cancer, skin cancer, malignant melanoma, endometrial cancer, esophageal cancer, gastric cancer, ovarian cancer, pancreatic cancer, or brain cancer.

In methods of detecting a tumor cfDNA provided herein, in some cases, the method comprises additional steps for addressing the cancer. In some embodiments, the method comprises recommending a treatment for the cancer in the subject. In some embodiments, the method comprises recommending administration of a chemotherapy to the subject. In some embodiments, the method comprises recommending additional cancer monitoring to the subject.

### Systems and Computer Assisted Methods

Provided herein are systems for practicing methods provided herein, including but not limited to methods of detecting non-metastatic cancer in a subject, methods of measuring cell-free deoxyribonucleic acid (cfDNA) size distribution in a sample from a subject, or detecting tumor cfDNA in a sample from a subject. A system herein may comprise a computer configured to receive a user request and an amplification system that performs a nucleic acid amplification reaction on cfDNA polynucleotides in the sample or a portion thereof in response to the user request. The system may further comprise a sequencing system that sequences the amplified cfDNA polynucleotides to produce a plurality of sequencing reads, determines a length for each individual polynucleotide of the plurality of cfDNA polynucleotides of the subject, and produces a total cfDNA fragment size distribution for a plurality of cfDNA polynucleotides in the sample. The system may further comprise a report generator that sends a report to a recipient. In more specific embodiments, systems herein may comprise a computer configured to receive a user request to perform a detection of non-metastatic cancer on a sample and an amplification system that performs a nucleic acid amplification reaction on cfDNA polynucleotides in the sample or a portion thereof in response to the user request. The system may further comprise a sequencing system that sequences the amplified cfDNA polynucleotides to produce a plurality of sequencing reads, determines a length for each individual polynucleotide of the plurality of cfDNA polynucleotides of the subject, and produces a total cfDNA fragment size distribution for a plurality of cfDNA polynucleotides in the sample. The system may additionally comprise a report generator that sends a report to a recipient, wherein the report contains results indicating a risk of non-metastatic cancer in the subject. In some embodiments, the method further comprises enriching the plurality of cfDNA polynucleotides for one or more target sequences. In some cases, the method does not comprise enriching for a target sequence. In some cases, the method does not comprise aligning or mapping a cfDNA polynucleotide sequence to a reference genome.

In systems for practicing methods provided herein, in certain embodiments, the system comprises an isolation system for isolating cfDNA polynucleotides from a blood sample of the subject. In some embodiments, the isolation system comprises a centrifuge. In some embodiments, the isolation system comprises a column, such as a nucleic acid binding column. In some embodiments, the isolation system comprises a filter. In some embodiments, the isolation system comprises a combination of the above features for isolating cfDNA from the blood sample of the subject.

In certain aspects of systems for practicing methods provided herein, the system may compare the total cfDNA fragment size distribution for the plurality of cfDNA polynucleotides of the subject to the total cfDNA fragment size distribution for the plurality of cfDNA polynucleotides from a healthy control. Systems provided herein may comprise a system for measuring cfDNA size using any suitable method. Suitable methods of measuring a cfDNA size distribution include, but are not limited to, sequencing, bioanalyzer fragment analysis, PCR, qPCR, high throughput gel electrophoresis, high throughput capillary electrophoresis, or any other suitable method that provides sizes of DNA fragments.

In systems provided herein, in certain cases, the plurality of cfDNA polynucleotides comprises only a small portion of circulating tumor deoxyribonucleic acid (ctDNA). For example, in some embodiments, the plurality of cfDNA polynucleotides comprises less than 25% ctDNA. In some embodiments, the plurality of cfDNA polynucleotides comprises less than 20% ctDNA. In some embodiments, the plurality of cfDNA polynucleotides comprises less than 15% ctDNA. In some embodiments, the plurality of cfDNA polynucleotides comprises less than 10% ctDNA. In some embodiments, the plurality of cfDNA polynucleotides comprises less than 5% ctDNA.

In some cases, systems provided herein comprise a module for preparation of a DNA library from polynucleotides in the sample of the subject. For example, in some embodiments, the method further comprises preparation of a single stranded DNA library from the cfDNA polynucleotides of the subject. Any suitable method of preparing a single stranded DNA library may be used in methods herein. For example, the method of preparing a single stranded DNA library may comprises denaturing the DNA sample to create a plurality of ssDNA; ligating an adapter to the 3′ end of the ssDNA molecules; synthesizing a second strand using a primer complementary to the adapter; ligating a double stranded adapter to the extension products; amplifying the second strand using primers targeting the first and second adapters (for example, using PCR); and sequencing the library on a sequencer. An additional method of single stranded library preparation may comprise denaturing the DNA sample to create a plurality of ssDNA; ligating an adapter to the 3′ end of the ssDNA molecules; synthesizing the second strand by using a primer complementary to the adapter; ligating a double stranded adapter to the extension products; amplifying the second strand (for example, by PCR) the second strand using primers targeting the first and second adapters; in some cases enriching for the regions of interest using hybridization with capture probes; amplifying (for example, by PCR) the captured products; and sequencing the library on a sequencer. The method may further comprise analyzing the resulting sequences (e.g., sequencing reads) using a sequence(s) of the primer(s) or capture probe(s), such as, for example, by grouping the resulting sequences (e.g., sequencing reads) according to the sequences of the primer(s) or capture probe(s)—for example, individual sequencing reads are distributed into individual groups each corresponding to a sequence of a primer or capture probe. This may be performed without aligning such resulting sequences with a genome.

The method may further comprise preparation of a double stranded DNA library from the cfDNA polynucleotides of the subject. Any suitable method of preparing a double stranded DNA library may be used in methods herein. For example, the method of preparing a double stranded DNA library may comprise ligating sequencing adapters to the 5′ and 3′ ends of a plurality of DNA fragments and sequencing the library on a sequencer. An additional method of double stranded DNA library preparation may comprise ligating adapters to the 5′ and 3′ ends of a plurality of DNA fragments; attaching the full adapter sequences to the ligated fragments through PCR using primers that are complementary to the ligated adapters; and sequencing the library on a sequencer. A further method may comprise ligating adapters to the 5′ and 3′ ends of a plurality of DNA fragments; amplifying the ligated product through PCR that are complementary to the ligated adapters; in some cases enriching for the regions of interest through hybridization with capture probes; PCR amplifying the captured products; and sequencing the library on a sequencer. An additional method of double stranded library preparation may comprise ligating adapters to the 5′ and 3′ ends of a plurality of DNA fragments; amplifying the ligated product through PCR using primers that are complementary to the ligated adapters; circularizing the double stranded PCR products or denature and circularize the single stranded PCR products; in some cases enriching for the regions of interest by PCR using primers targeting specific genes; and sequencing the library on a sequencer. The method may further comprise analyzing the resulting sequences (e.g., sequencing reads) using a sequence(s) of the primer(s) or capture probe(s), such as, for example, by grouping the resulting sequences (e.g., sequencing reads) according to the sequences of the primer(s) or capture probe(s)—for example, individual sequencing reads are distributed into individual groups each corresponding to a sequence of a primer or capture probe. This may be performed without aligning such resulting sequences with a genome.

Systems provided herein may further comprise modules for amplification and sequencing steps including one or more modules for circularizing individual cfDNA polynucleotides of the plurality of cfDNA polynucleotides of the subject to form a plurality of circular polynucleotides, amplifying the circular polynucleotides, sequencing the amplified polynucleotides to produce a plurality of sequencing reads and determining a length for each individual cfDNA polynucleotide of the plurality of cfDNA polynucleotides of the subject. Systems herein, in some embodiments, do not comprise isolating tumor cfDNA polynucleotides from total cfDNA polynucleotides.

In some cases, systems provided herein detect a non-metastatic cancer in the subject when the cfDNA fragment size distribution shows an increase in fragments having sizes of 10 bases to 170 bases in the subject compared to the healthy control. In some embodiments, the cfDNA fragment size distribution shows at least an increase in fragments having sizes of 10 bases to 50 bases, 10 bases to 70 bases, 10 bases to 105 bases, 10 bases to 115 bases, 10 bases to 125 bases, 10 bases to 170 bases, 25 bases to 50 bases, 25 bases to 70 bases, 25 bases to 105 bases, 25 bases to 115 bases, 25 bases to 125 bases, 25 bases to 170 bases, 50 bases to 70 bases, 50 bases to 105 bases, 50 bases to 115 bases, 50 bases to 125 bases, 50 bases to 170 bases, 70 bases to 105 bases, 70 bases to 115 bases, 70 bases to 125 bases, 70 bases to 170 bases, 105 bases to 115 bases, 105 bases to 125 bases, 105 bases to 170 bases, 115 bases to 125 bases, 115 bases to 170 bases, or 125 bases to 170 bases in the subject compared to the healthy control.

In some cases, systems provided herein detect a non-metastatic cancer in the subject when the cfDNA fragment size distribution shows an increase in fragments having sizes of 10 bases to 170 bases in the subject compared to the healthy control. Alternately or in combination, the report generator classifies the subject as having an increased risk of non-metastatic cancer when the cfDNA fragment size distribution shows an increase in fragments having sizes of 10 bases to 170 bases in the subject compared to the healthy control. In some embodiments of systems provided herein, the cfDNA fragment size distribution shows at least a 0.25% increase in fragments having sizes of 10 bases to 170 bases in the subject compared to the healthy control. In some embodiments, the cfDNA fragment size distribution shows at least a 0.5% increase in fragments having sizes of 10 bases to 170 bases in the subject compared to the healthy control. In some embodiments, the cfDNA fragment size distribution shows at least a 1% increase in fragments having sizes of 10 bases to 170 bases in the subject compared to the healthy control. In some embodiments, the cfDNA fragment size distribution shows at least a 2% increase in fragments having sizes of 10 bases to 170 bases in the subject compared to the healthy control. In some embodiments, the cfDNA fragment size distribution shows at least a 3% increase in fragments having sizes of 10 bases to 170 bases in the subject compared to the healthy control. In some embodiments, the cfDNA fragment size distribution shows at least a 4% increase in fragments having sizes of 10 bases to 170 bases in the subject compared to the healthy control. In some embodiments, the cfDNA fragment size distribution shows at least a 5% increase in fragments having sizes of 10 bases to 170 bases in the subject compared to the healthy control. In some embodiments, the cfDNA fragment size distribution shows at least a 6% increase in fragments having sizes of 10 bases to 170 bases in the subject compared to the healthy control. In some embodiments, the cfDNA fragment size distribution shows at least a 7% increase in fragments having sizes of 10 bases to 170 bases in the subject compared to the healthy control. In some embodiments, the cfDNA fragment size distribution shows at least a 8% increase in fragments having sizes of 10 bases to 170 bases in the subject compared to the healthy control. In some embodiments, the cfDNA fragment size distribution shows at least a 9% increase in fragments having sizes of 10 bases to 170 bases in the subject compared to the healthy control. In some embodiments, the cfDNA fragment size distribution shows at least a 10% increase in fragments having sizes of 10 bases to 170 bases in the subject compared to the healthy control. In some embodiments, the cfDNA fragment size distribution shows at least a 15% increase in fragments having sizes of 10 bases to 170 bases in the subject compared to the healthy control. In some embodiments, the cfDNA fragment size distribution shows at least a 20% increase in fragments having sizes of 10 bases to 170 bases in the subject compared to the healthy control.

In some cases, systems provided herein detect a non-metastatic cancer in the subject when the cfDNA fragment size distribution shows an increase in fragments having sizes of 50 bases to 170 bases in the subject compared to the healthy control. Alternately or in combination, the report generator classifies the subject as having an increased risk of non-metastatic cancer when the cfDNA fragment size distribution shows an increase in fragments having sizes of 50 bases to 170 bases in the subject compared to the healthy control. In some embodiments of systems provided herein, the cfDNA fragment size distribution shows at least a 0.25% increase in fragments having sizes of 50 bases to 170 bases in the subject compared to the healthy control. In some embodiments, the cfDNA fragment size distribution shows at least a 0.5% increase in fragments having sizes of 50 bases to 170 bases in the subject compared to the healthy control. In some embodiments, the cfDNA fragment size distribution shows at least a 1% increase in fragments having sizes of 50 bases to 170 bases in the subject compared to the healthy control. In some embodiments, the cfDNA fragment size distribution shows at least a 2% increase in fragments having sizes of 50 bases to 170 bases in the subject compared to the healthy control. In some embodiments, the cfDNA fragment size distribution shows at least a 3% increase in fragments having sizes of 50 bases to 170 bases in the subject compared to the healthy control. In some embodiments, the cfDNA fragment size distribution shows at least a 4% increase in fragments having sizes of 50 bases to 170 bases in the subject compared to the healthy control. In some embodiments, the cfDNA fragment size distribution shows at least a 5% increase in fragments having sizes of 50 bases to 170 bases in the subject compared to the healthy control. In some embodiments, the cfDNA fragment size distribution shows at least a 6% increase in fragments having sizes of 50 bases to 170 bases in the subject compared to the healthy control. In some embodiments, the cfDNA fragment size distribution shows at least a 7% increase in fragments having sizes of 50 bases to 170 bases in the subject compared to the healthy control. In some embodiments, the cfDNA fragment size distribution shows at least a 8% increase in fragments having sizes of 50 bases to 170 bases in the subject compared to the healthy control. In some embodiments, the cfDNA fragment size distribution shows at least a 9% increase in fragments having sizes of 50 bases to 170 bases in the subject compared to the healthy control. In some embodiments, the cfDNA fragment size distribution shows at least a 10% increase in fragments having sizes of 50 bases to 170 bases in the subject compared to the healthy control. In some embodiments, the cfDNA fragment size distribution shows at least a 15% increase in fragments having sizes of 50 bases to 170 bases in the subject compared to the healthy control. In some embodiments, the cfDNA fragment size distribution shows at least a 20% increase in fragments having sizes of 50 bases to 170 bases in the subject compared to the healthy control.

Further provided herein are systems for detecting a non-metastatic cancer in the subject comprising a computer configured to receive a user request and an amplification system that performs a nucleic acid amplification reaction on cfDNA polynucleotides in the sample or a portion thereof in response to the user request. The system may further comprise a sequencing system that sequences the amplified cfDNA polynucleotides to produce a plurality of sequencing reads, determines a length for each individual polynucleotide of the plurality of cfDNA polynucleotides of the subject, and produces a total cfDNA fragment size distribution for a plurality of cfDNA polynucleotides in the sample. The system may further comprise a report generator that sends a report to a recipient. In more specific embodiments, systems herein may comprise a computer configured to receive a user request to perform a detection of non-metastatic cancer on a sample and an amplification system that performs a nucleic acid amplification reaction on cfDNA polynucleotides in the sample or a portion thereof in response to the user request. The system may further comprise a sequencing system that sequences the amplified cfDNA polynucleotides to produce a plurality of sequencing reads, determines a length for each individual polynucleotide of the plurality of cfDNA polynucleotides of the subject, and produces a total cfDNA fragment size distribution for a plurality of cfDNA polynucleotides in the sample. The system may further comprise a report generator that sends a report to a recipient, wherein the report contains results indicating a risk of non-metastatic cancer in the subject; wherein the report classifies the subject as having an increased risk of non-metastatic cancer when the cfDNA fragment size distribution shows an increase in the subject compared to the healthy control in at least 2, 3, 4, 5, 6, 7, 8, 9, 10, or more cfDNA fragments having sizes in a range of 10 bases to 170 bases.

In certain systems provided herein, the system may classify the subject as having an increased risk of non-metastatic cancer when the cfDNA fragment size distribution shows an increase in fragments having sizes of 50 bases to 125 bases in the subject compared to the healthy control. In some embodiments, the cfDNA fragment size distribution shows at least a 0.25% increase in fragments having sizes of 50 bases to 125 bases in the subject compared to the healthy control. In some embodiments, the cfDNA fragment size distribution shows at least a 0.5% increase in fragments having sizes of 50 bases to 125 bases in the subject compared to the healthy control. In some embodiments, the cfDNA fragment size distribution shows at least a 1% increase in fragments having sizes of 50 bases to 125 bases in the subject compared to the healthy control. In some embodiments, the cfDNA fragment size distribution shows at least a 2% increase in fragments having sizes of 50 bases to 125 bases in the subject compared to the healthy control. In some embodiments, the cfDNA fragment size distribution shows at least a 3% increase in fragments having sizes of 50 bases to 125 bases in the subject compared to the healthy control. In some embodiments, the cfDNA fragment size distribution shows at least a 4% increase in fragments having sizes of 50 bases to 125 bases in the subject compared to the healthy control. In some embodiments, the cfDNA fragment size distribution shows at least a 5% increase in fragments having sizes of 50 bases to 125 bases in the subject compared to the healthy control. In some embodiments, the cfDNA fragment size distribution shows at least a 6% increase in fragments having sizes of 50 bases to 125 bases in the subject compared to the healthy control. In some embodiments, the cfDNA fragment size distribution shows at least a 7% increase in fragments having sizes of 50 bases to 125 bases in the subject compared to the healthy control. In some embodiments, the cfDNA fragment size distribution shows at least a 8% increase in fragments having sizes of 50 bases to 125 bases in the subject compared to the healthy control. In some embodiments, the cfDNA fragment size distribution shows at least a 9% increase in fragments having sizes of 50 bases to 125 bases in the subject compared to the healthy control. In some embodiments, the cfDNA fragment size distribution shows at least a 10% increase in fragments having sizes of 50 bases to 125 bases in the subject compared to the healthy control. In some embodiments, the cfDNA fragment size distribution shows at least a 15% increase in fragments having sizes of 50 bases to 125 bases in the subject compared to the healthy control. In some embodiments, the cfDNA fragment size distribution shows at least a 20% increase in fragments having sizes of 50 bases to 125 bases in the subject compared to the healthy control.

In certain systems provided herein, the system may classify the subject as having an increased risk of non-metastatic cancer when the cfDNA fragment size distribution shows an increase in fragments having sizes of 50 bases to 115 bases in the subject compared to the healthy control. In some embodiments, the cfDNA fragment size distribution shows at least a 0.25% increase in fragments having sizes of 50 bases to 115 bases in the subject compared to the healthy control. In some embodiments, the cfDNA fragment size distribution shows at least a 0.5% increase in fragments having sizes of 50 bases to 115 bases in the subject compared to the healthy control. In some embodiments, the cfDNA fragment size distribution shows at least a 1% increase in fragments having sizes of 50 bases to 115 bases in the subject compared to the healthy control. In some embodiments, the cfDNA fragment size distribution shows at least a 2% increase in fragments having sizes of 50 bases to 115 bases in the subject compared to the healthy control. In some embodiments, the cfDNA fragment size distribution shows at least a 3% increase in fragments having sizes of 50 bases to 115 bases in the subject compared to the healthy control. In some embodiments, the cfDNA fragment size distribution shows at least a 4% increase in fragments having sizes of 50 bases to 115 bases in the subject compared to the healthy control. In some embodiments, the cfDNA fragment size distribution shows at least a 5% increase in fragments having sizes of 50 bases to 115 bases in the subject compared to the healthy control. In some embodiments, the cfDNA fragment size distribution shows at least a 6% increase in fragments having sizes of 50 bases to 115 bases in the subject compared to the healthy control. In some embodiments, the cfDNA fragment size distribution shows at least a 7% increase in fragments having sizes of 50 bases to 115 bases in the subject compared to the healthy control. In some embodiments, the cfDNA fragment size distribution shows at least a 8% increase in fragments having sizes of 50 bases to 115 bases in the subject compared to the healthy control. In some embodiments, the cfDNA fragment size distribution shows at least a 9% increase in fragments having sizes of 50 bases to 115 bases in the subject compared to the healthy control. In some embodiments, the cfDNA fragment size distribution shows at least a 10% increase in fragments having sizes of 50 bases to 115 bases in the subject compared to the healthy control. In some embodiments, the cfDNA fragment size distribution shows at least a 15% increase in fragments having sizes of 50 bases to 115 bases in the subject compared to the healthy control. In some embodiments, the cfDNA fragment size distribution shows at least a 20% increase in fragments having sizes of 50 bases to 115 bases in the subject compared to the healthy control.

In certain systems provided herein, the system may classify the subject as having an increased risk of non-metastatic cancer when the cfDNA fragment size distribution shows an increase in fragments having sizes of 50 bases to 105 bases in the subject compared to the healthy control. In some embodiments, the cfDNA fragment size distribution shows at least a 0.25% increase in fragments having sizes of 50 bases to 105 bases in the subject compared to the healthy control. In some embodiments, the cfDNA fragment size distribution shows at least a 0.5% increase in fragments having sizes of 50 bases to 105 bases in the subject compared to the healthy control. In some embodiments, the cfDNA fragment size distribution shows at least a 1% increase in fragments having sizes of 50 bases to 105 bases in the subject compared to the healthy control. In some embodiments, the cfDNA fragment size distribution shows at least a 2% increase in fragments having sizes of 50 bases to 105 bases in the subject compared to the healthy control. In some embodiments, the cfDNA fragment size distribution shows at least a 3% increase in fragments having sizes of 50 bases to 105 bases in the subject compared to the healthy control. In some embodiments, the cfDNA fragment size distribution shows at least a 4% increase in fragments having sizes of 50 bases to 105 bases in the subject compared to the healthy control. In some embodiments, the cfDNA fragment size distribution shows at least a 5% increase in fragments having sizes of 50 bases to 105 bases in the subject compared to the healthy control. In some embodiments, the cfDNA fragment size distribution shows at least a 6% increase in fragments having sizes of 50 bases to 105 bases in the subject compared to the healthy control. In some embodiments, the cfDNA fragment size distribution shows at least a 7% increase in fragments having sizes of 50 bases to 105 bases in the subject compared to the healthy control. In some embodiments, the cfDNA fragment size distribution shows at least a 8% increase in fragments having sizes of 50 bases to 105 bases in the subject compared to the healthy control. In some embodiments, the cfDNA fragment size distribution shows at least a 9% increase in fragments having sizes of 50 bases to 105 bases in the subject compared to the healthy control. In some embodiments, the cfDNA fragment size distribution shows at least a 10% increase in fragments having sizes of 50 bases to 105 bases in the subject compared to the healthy control. In some embodiments, the cfDNA fragment size distribution shows at least a 15% increase in fragments having sizes of 50 bases to 105 bases in the subject compared to the healthy control. In some embodiments, the cfDNA fragment size distribution shows at least a 20% increase in fragments having sizes of 50 bases to 105 bases in the subject compared to the healthy control.

In certain aspects of systems provided herein, the subject may have cancer but in the very early stages, in some instances, before it can be detected using conventional methods. In some cases, the subject does not have a diagnosis of metastatic cancer. Alternatively, the subject has a low tumor burden, for example, in some cases, the subject has a tumor burden of less than 20%. In some embodiments, the subject has a tumor burden of less than 10%. In some embodiments, the subject has a tumor burden of less than 9%. In some embodiments, the subject has a tumor burden of less than 8%. In some embodiments, the subject has a tumor burden of less than 7%. In some embodiments, the subject has a tumor burden of less than 6%. In some embodiments, the subject has a tumor burden of less than 5%. Alternatively, the early stage cancers detected using methods herein comprise a non-metastatic cancer having an early stage, which may be classified using a numerical classification system. In some cases, an early stage cancer is staged according to the type of cancer. In some embodiments, the non-metastatic cancer is stage 0, stage 1, stage 2, or stage 3.

Cancers and tumor cfDNA detectable using systems herein may include, but are not limited to, colon cancer, non-small cell lung cancer, small cell lung cancer, breast cancer, hepatocellular carcinoma, liver cancer, skin cancer, malignant melanoma, endometrial cancer, esophageal cancer, gastric cancer, ovarian cancer, pancreatic cancer, or brain cancer.

In systems provided herein, in some cases, the system prepares a report containing additional steps for addressing the cancer. In some embodiments, the report recommends a treatment for the cancer in the subject. In some embodiments, the report recommends administration of a chemotherapy to the subject. In some embodiments, the report recommends additional cancer monitoring to the subject.

Additionally provided herein are computer assisted methods such as computer-readable media comprising codes that, upon execution by one or more processors, may implement methods provided herein, including, but not limited to, methods of detecting non-metastatic cancer in a subject, methods of measuring cfDNA size distribution in a sample from a subject, or detecting tumor cfDNA in a sample from a subject. In some embodiments, there are provided computer-readable media comprising codes that, upon execution by one or more processors, implement a method comprising receiving a customer request to perform a detection or measurement on a sample from a subject and performing a nucleic acid amplification reaction on cfDNA polynucleotides in the sample or a portion thereof. Next, a sequencing analysis is performed comprising the operations of sequencing the amplified cfDNA polynucleotides to produce a plurality of sequencing reads; determining a length for each individual polynucleotide of the plurality of cfDNA polynucleotides of the subject; and producing a total cfDNA fragment size distribution for a plurality of cfDNA polynucleotides in the sample. In some cases, a report is generated. In more specific embodiments, there are provided computer-readable media comprising codes that, upon execution by one or more processors, implement a method of detecting a non-metastatic cancer in a subject comprising receiving a customer request to perform a detection of non-metastatic cancer on a sample from a subject and performing a nucleic acid amplification reaction on cfDNA polynucleotides in the sample or a portion thereof. Next, a sequencing analysis may be performed comprising the operations of sequencing the amplified cfDNA polynucleotides to produce a plurality of sequencing reads; determining a length for each individual polynucleotide of the plurality of cfDNA polynucleotides of the subject; and producing a total cfDNA fragment size distribution for a plurality of cfDNA polynucleotides in the sample. In some cases, a report is generated that contains results indicating a risk of non-metastatic cancer in the subject.

In computer-readable media for practicing methods provided herein the computer-readable media may implement an isolation system for isolating cfDNA polynucleotides from a blood sample of the subject. In some embodiments, the isolation system comprises a centrifuge. In some embodiments, the isolation system comprises a column, such as a nucleic acid binding column. In some embodiments, the isolation system comprises a filter. In some embodiments, the isolation system comprises a combination of the above features for isolating cfDNA from the blood sample of the subject. In some embodiments, the method further comprises enriching the plurality of cfDNA polynucleotides for one or more target sequences. In some cases, the method does not comprise enriching for a target sequence. In some cases, the method does not comprise aligning or mapping a cfDNA polynucleotide sequence to a reference genome.

In certain aspects of computer-readable media for practicing methods provided herein, the method may comprise comparing the total cfDNA fragment size distribution for the plurality of cfDNA polynucleotides of the subject to the total cfDNA fragment size distribution for the plurality of cfDNA polynucleotides from a healthy control. Methods of measuring a cfDNA size distribution provided herein may comprise any suitable method of measuring DNA size. Suitable methods of measuring a cfDNA size distribution may include, but are not limited to, sequencing, bioanalyzer fragment analysis, PCR, qPCR, high throughput gel electrophoresis, high throughput capillary electrophoresis, or any other suitable method that provides sizes of DNA fragments.

In some cases, computer-readable media provided herein implement a method for preparation of a DNA library from polynucleotides in the sample of the subject. For example, the method may further comprise preparation of a single stranded DNA library from the cfDNA polynucleotides of the subject. Any suitable method of preparing a single stranded DNA library may be used in methods herein. For example, the method of preparing a single stranded DNA library may comprise denaturing the DNA sample to create a plurality of ssDNA; ligating an adapter to the 3′ end of the ssDNA molecules; synthesizing a second strand using a primer complementary to the adapter; ligating a double stranded adapter to the extension products; amplifying the second strand using primers targeting the first and second adapters (for example, using PCR); and sequencing the library on a sequencer. An additional method of single stranded library preparation may comprise denaturing the DNA sample to create a plurality of ssDNA; ligating an adapter to the 3′ end of the ssDNA molecules; synthesizing the second strand by using a primer complementary to the adapter; ligating a double stranded adapter to the extension products; amplifying the second strand (for example, by PCR) the second strand using primers targeting the first and second adapters; in some cases enriching for the regions of interest using hybridization with capture probes; amplifying (for example, by PCR) the captured products; and sequencing the library on a sequencer. The method may further comprise analyzing the resulting sequences (e.g., sequencing reads) using a sequence(s) of the primer(s) or capture probe(s), such as, for example, by grouping the resulting sequences (e.g., sequencing reads) according to the sequences of the primer(s) or capture probe(s)—for example, individual sequencing reads are distributed into individual groups each corresponding to a sequence of a primer or capture probe. This may be performed without aligning such resulting sequences with a genome.

Alternatively or in combination, the method may further comprise preparation of a double stranded DNA library from the cfDNA polynucleotides of the subject. Any suitable method of preparing a double stranded DNA library may be used in methods herein. For example, the method of preparing a double stranded DNA library may comprise ligating sequencing adapters to the 5′ and 3′ ends of a plurality of DNA fragments and sequencing the library on a sequencer. An additional method of double stranded DNA library preparation may comprise ligating adapters to the 5′ and 3′ ends of a plurality of DNA fragments; attaching the full adapter sequences to the ligated fragments through PCR using primers that are complementary to the ligated adapters; and sequencing the library on a sequencer. A further method may comprise ligating adapters to the 5′ and 3′ ends of a plurality of DNA fragments; amplifying the ligated product through PCR that are complementary to the ligated adapters; in some cases enriching for the regions of interest through hybridization with capture probes; PCR amplifying the captured products; and sequencing the library on a sequencer. An additional method of double stranded library preparation may comprise ligating adapters to the 5′ and 3′ ends of a plurality of DNA fragments; amplifying the ligated product through PCR using primers that are complementary to the ligated adapters; circularizing the double stranded PCR products or denature and circularize the single stranded PCR products; in some cases enriching for the regions of interest by PCR using primers targeting specific genes; and sequencing the library on a sequencer. The method may further comprise analyzing the resulting sequences (e.g., sequencing reads) using a sequence(s) of the primer(s) or capture probe(s), such as, for example, by grouping the resulting sequences (e.g., sequencing reads) according to the sequences of the primer(s) or capture probe(s)—for example, individual sequencing reads are distributed into individual groups each corresponding to a sequence of a primer or capture probe. This may be performed without aligning such resulting sequences with a genome.

Computer-readable media herein may implement methods for amplification and sequencing steps including one or more modules for circularizing individual cfDNA polynucleotides of the plurality of cfDNA polynucleotides of the subject to form a plurality of circular polynucleotides, amplifying the circular polynucleotides, sequencing the amplified polynucleotides to produce a plurality of sequencing reads, and determining a length for each individual cfDNA polynucleotide of the plurality of cfDNA polynucleotides of the subject. Computer-readable media herein, in some embodiments, do not comprise isolating tumor cfDNA polynucleotides from total cfDNA polynucleotides.

In computer-readable media provided herein, the plurality of cfDNA polynucleotides may comprise only a small portion of ctDNA. For example, in some embodiments, the plurality of cfDNA polynucleotides comprises less than 25% ctDNA. In some embodiments, the plurality of cfDNA polynucleotides comprises less than 20% ctDNA. In some embodiments, the plurality of cfDNA polynucleotides comprises less than 15% ctDNA. In some embodiments, the plurality of cfDNA polynucleotides comprises less than 10% ctDNA. In some embodiments, the plurality of cfDNA polynucleotides comprises less than 5% ctDNA.

In some cases, computer-readable media provided herein implement a method of detecting a non-metastatic cancer in the subject when the cfDNA fragment size distribution shows an increase in fragments having sizes of 10 bases to 170 bases in the subject compared to the healthy control. In some embodiments, the cfDNA fragment size distribution shows at least an increase in fragments having sizes of 10 bases to 50 bases, 10 bases to 70 bases, 10 bases to 105 bases, 10 bases to 115 bases, 10 bases to 125 bases, 10 bases to 170 bases, 25 bases to 50 bases, 25 bases to 70 bases, 25 bases to 105 bases, 25 bases to 115 bases, 25 bases to 125 bases, 25 bases to 170 bases, 50 bases to 70 bases, 50 bases to 105 bases, 50 bases to 115 bases, 50 bases to 125 bases, 50 bases to 170 bases, 70 bases to 105 bases, 70 bases to 115 bases, 70 bases to 125 bases, 70 bases to 170 bases, 105 bases to 115 bases, 105 bases to 125 bases, 105 bases to 170 bases, 115 bases to 125 bases, 115 bases to 170 bases, or 125 bases to 170 bases in the subject compared to the healthy control.

In some cases, computer-readable media provided herein implement a method of detecting a non-metastatic cancer in the subject when the cfDNA fragment size distribution shows an increase in fragments having sizes of 10 bases to 170 bases in the subject compared to the healthy control. Alternately or in combination, the method may classify the subject as having an increased risk of non-metastatic cancer when the cfDNA fragment size distribution shows an increase in fragments having sizes of 10 bases to 170 bases in the subject compared to the healthy control. In some embodiments of computer-readable media provided herein, the cfDNA fragment size distribution shows at least a 0.25% increase in fragments having sizes of 10 bases to 170 bases in the subject compared to the healthy control. In some embodiments, the cfDNA fragment size distribution shows at least a 0.5% increase in fragments having sizes of 10 bases to 170 bases in the subject compared to the healthy control. In some embodiments, the cfDNA fragment size distribution shows at least a 1% increase in fragments having sizes of 10 bases to 170 bases in the subject compared to the healthy control. In some embodiments, the cfDNA fragment size distribution shows at least a 2% increase in fragments having sizes of 10 bases to 170 bases in the subject compared to the healthy control. In some embodiments, the cfDNA fragment size distribution shows at least a 3% increase in fragments having sizes of 10 bases to 170 bases in the subject compared to the healthy control. In some embodiments, the cfDNA fragment size distribution shows at least a 4% increase in fragments having sizes of 10 bases to 170 bases in the subject compared to the healthy control. In some embodiments, the cfDNA fragment size distribution shows at least a 5% increase in fragments having sizes of 10 bases to 170 bases in the subject compared to the healthy control. In some embodiments, the cfDNA fragment size distribution shows at least a 6% increase in fragments having sizes of 10 bases to 170 bases in the subject compared to the healthy control. In some embodiments, the cfDNA fragment size distribution shows at least a 7% increase in fragments having sizes of 10 bases to 170 bases in the subject compared to the healthy control. In some embodiments, the cfDNA fragment size distribution shows at least a 8% increase in fragments having sizes of 10 bases to 170 bases in the subject compared to the healthy control. In some embodiments, the cfDNA fragment size distribution shows at least a 9% increase in fragments having sizes of 10 bases to 170 bases in the subject compared to the healthy control. In some embodiments, the cfDNA fragment size distribution shows at least a 10% increase in fragments having sizes of 10 bases to 170 bases in the subject compared to the healthy control. In some embodiments, the cfDNA fragment size distribution shows at least a 15% increase in fragments having sizes of 10 bases to 170 bases in the subject compared to the healthy control. In some embodiments, the cfDNA fragment size distribution shows at least a 20% increase in fragments having sizes of 10 bases to 170 bases in the subject compared to the healthy control.

In some cases, computer-readable media provided herein implement a method of detecting a non-metastatic cancer in the subject when the cfDNA fragment size distribution shows an increase in fragments having sizes of 50 bases to 170 bases in the subject compared to the healthy control. Alternately or in combination, the method may classify the subject as having an increased risk of non-metastatic cancer when the cfDNA fragment size distribution shows an increase in fragments having sizes of 50 bases to 170 bases in the subject compared to the healthy control. In some embodiments of computer-readable media provided herein, the cfDNA fragment size distribution shows at least a 0.25% increase in fragments having sizes of 50 bases to 170 bases in the subject compared to the healthy control. In some embodiments, the cfDNA fragment size distribution shows at least a 0.5% increase in fragments having sizes of 50 bases to 170 bases in the subject compared to the healthy control. In some embodiments, the cfDNA fragment size distribution shows at least a 1% increase in fragments having sizes of 50 bases to 170 bases in the subject compared to the healthy control. In some embodiments, the cfDNA fragment size distribution shows at least a 2% increase in fragments having sizes of 50 bases to 170 bases in the subject compared to the healthy control. In some embodiments, the cfDNA fragment size distribution shows at least a 3% increase in fragments having sizes of 50 bases to 170 bases in the subject compared to the healthy control. In some embodiments, the cfDNA fragment size distribution shows at least a 4% increase in fragments having sizes of 50 bases to 170 bases in the subject compared to the healthy control. In some embodiments, the cfDNA fragment size distribution shows at least a 5% increase in fragments having sizes of 50 bases to 170 bases in the subject compared to the healthy control. In some embodiments, the cfDNA fragment size distribution shows at least a 6% increase in fragments having sizes of 50 bases to 170 bases in the subject compared to the healthy control. In some embodiments, the cfDNA fragment size distribution shows at least a 7% increase in fragments having sizes of 50 bases to 170 bases in the subject compared to the healthy control. In some embodiments, the cfDNA fragment size distribution shows at least a 8% increase in fragments having sizes of 50 bases to 170 bases in the subject compared to the healthy control. In some embodiments, the cfDNA fragment size distribution shows at least a 9% increase in fragments having sizes of 50 bases to 170 bases in the subject compared to the healthy control. In some embodiments, the cfDNA fragment size distribution shows at least a 10% increase in fragments having sizes of 50 bases to 170 bases in the subject compared to the healthy control. In some embodiments, the cfDNA fragment size distribution shows at least a 15% increase in fragments having sizes of 50 bases to 170 bases in the subject compared to the healthy control. In some embodiments, the cfDNA fragment size distribution shows at least a 20% increase in fragments having sizes of 50 bases to 170 bases in the subject compared to the healthy control.

In some embodiments, there are provided computer-readable media comprising codes that, upon execution by one or more processors, implement a method comprising receiving a customer request to perform a detection or measurement on a sample from a subject and performing a nucleic acid amplification reaction on cfDNA polynucleotides in the sample or a portion thereof. Next, a sequencing analysis may be performed comprising the operations of sequencing the amplified cfDNA polynucleotides to produce a plurality of sequencing reads, determining a length for each individual polynucleotide of the plurality of cfDNA polynucleotides of the subject, and producing a total cfDNA fragment size distribution for a plurality of cfDNA polynucleotides in the sample. A report may be generated, wherein the report classifies the subject as having an increased risk of non-metastatic cancer when the cfDNA fragment size distribution shows an increase in the subject compared to the healthy control in at least 2, 3, 4, 5, 6, 7, 8, 9, 10, or more cfDNA fragments having sizes in a range of 10 bases to 170 bases.

Computer-readable media provided herein may implement a method of classifying the subject as having an increased risk of non-metastatic cancer when the cfDNA fragment size distribution shows an increase in fragments having sizes of 50 bases to 125 bases in the subject compared to the healthy control. In some embodiments, the cfDNA fragment size distribution shows at least a 0.25% increase in fragments having sizes of 50 bases to 125 bases in the subject compared to the healthy control. In some embodiments, the cfDNA fragment size distribution shows at least a 0.5% increase in fragments having sizes of 50 bases to 125 bases in the subject compared to the healthy control. In some embodiments, the cfDNA fragment size distribution shows at least a 1% increase in fragments having sizes of 50 bases to 125 bases in the subject compared to the healthy control. In some embodiments, the cfDNA fragment size distribution shows at least a 2% increase in fragments having sizes of 50 bases to 125 bases in the subject compared to the healthy control. In some embodiments, the cfDNA fragment size distribution shows at least a 3% increase in fragments having sizes of 50 bases to 125 bases in the subject compared to the healthy control. In some embodiments, the cfDNA fragment size distribution shows at least a 4% increase in fragments having sizes of 50 bases to 125 bases in the subject compared to the healthy control. In some embodiments, the cfDNA fragment size distribution shows at least a 5% increase in fragments having sizes of 50 bases to 125 bases in the subject compared to the healthy control. In some embodiments, the cfDNA fragment size distribution shows at least a 6% increase in fragments having sizes of 50 bases to 125 bases in the subject compared to the healthy control. In some embodiments, the cfDNA fragment size distribution shows at least a 7% increase in fragments having sizes of 50 bases to 125 bases in the subject compared to the healthy control. In some embodiments, the cfDNA fragment size distribution shows at least a 8% increase in fragments having sizes of 50 bases to 125 bases in the subject compared to the healthy control. In some embodiments, the cfDNA fragment size distribution shows at least a 9% increase in fragments having sizes of 50 bases to 125 bases in the subject compared to the healthy control. In some embodiments, the cfDNA fragment size distribution shows at least a 10% increase in fragments having sizes of 50 bases to 125 bases in the subject compared to the healthy control. In some embodiments, the cfDNA fragment size distribution shows at least a 15% increase in fragments having sizes of 50 bases to 125 bases in the subject compared to the healthy control. In some embodiments, the cfDNA fragment size distribution shows at least a 20% increase in fragments having sizes of 50 bases to 125 bases in the subject compared to the healthy control.

Computer-readable media provided herein may implement a method of classifying the subject as having an increased risk of non-metastatic cancer when the cfDNA fragment size distribution shows an increase in fragments having sizes of 50 bases to 115 bases in the subject compared to the healthy control. In some embodiments, the cfDNA fragment size distribution shows at least a 0.25% increase in fragments having sizes of 50 bases to 115 bases in the subject compared to the healthy control. In some embodiments, the cfDNA fragment size distribution shows at least a 0.5% increase in fragments having sizes of 50 bases to 115 bases in the subject compared to the healthy control. In some embodiments, the cfDNA fragment size distribution shows at least a 1% increase in fragments having sizes of 50 bases to 115 bases in the subject compared to the healthy control. In some embodiments, the cfDNA fragment size distribution shows at least a 2% increase in fragments having sizes of 50 bases to 115 bases in the subject compared to the healthy control. In some embodiments, the cfDNA fragment size distribution shows at least a 3% increase in fragments having sizes of 50 bases to 115 bases in the subject compared to the healthy control. In some embodiments, the cfDNA fragment size distribution shows at least a 4% increase in fragments having sizes of 50 bases to 115 bases in the subject compared to the healthy control. In some embodiments, the cfDNA fragment size distribution shows at least a 5% increase in fragments having sizes of 50 bases to 115 bases in the subject compared to the healthy control. In some embodiments, the cfDNA fragment size distribution shows at least a 6% increase in fragments having sizes of 50 bases to 115 bases in the subject compared to the healthy control. In some embodiments, the cfDNA fragment size distribution shows at least a 7% increase in fragments having sizes of 50 bases to 115 bases in the subject compared to the healthy control. In some embodiments, the cfDNA fragment size distribution shows at least an 8% increase in fragments having sizes of 50 bases to 115 bases in the subject compared to the healthy control. In some embodiments, the cfDNA fragment size distribution shows at least a 9% increase in fragments having sizes of 50 bases to 115 bases in the subject compared to the healthy control. In some embodiments, the cfDNA fragment size distribution shows at least a 10% increase in fragments having sizes of 50 bases to 115 bases in the subject compared to the healthy control. In some embodiments, the cfDNA fragment size distribution shows at least a 15% increase in fragments having sizes of 50 bases to 115 bases in the subject compared to the healthy control. In some embodiments, the cfDNA fragment size distribution shows at least a 20% increase in fragments having sizes of 50 bases to 115 bases in the subject compared to the healthy control.

Computer-readable media provided herein may implement a method of classifying the subject as having an increased risk of non-metastatic cancer when the cfDNA fragment size distribution shows an increase in fragments having sizes of 50 bases to 105 bases in the subject compared to the healthy control. In some embodiments, the cfDNA fragment size distribution shows at least a 0.25% increase in fragments having sizes of 50 bases to 105 bases in the subject compared to the healthy control. In some embodiments, the cfDNA fragment size distribution shows at least a 0.5% increase in fragments having sizes of 50 bases to 105 bases in the subject compared to the healthy control. In some embodiments, the cfDNA fragment size distribution shows at least a 1% increase in fragments having sizes of 50 bases to 105 bases in the subject compared to the healthy control. In some embodiments, the cfDNA fragment size distribution shows at least a 2% increase in fragments having sizes of 50 bases to 105 bases in the subject compared to the healthy control. In some embodiments, the cfDNA fragment size distribution shows at least a 3% increase in fragments having sizes of 50 bases to 105 bases in the subject compared to the healthy control. In some embodiments, the cfDNA fragment size distribution shows at least a 4% increase in fragments having sizes of 50 bases to 105 bases in the subject compared to the healthy control. In some embodiments, the cfDNA fragment size distribution shows at least a 5% increase in fragments having sizes of 50 bases to 105 bases in the subject compared to the healthy control. In some embodiments, the cfDNA fragment size distribution shows at least a 6% increase in fragments having sizes of 50 bases to 105 bases in the subject compared to the healthy control. In some embodiments, the cfDNA fragment size distribution shows at least a 7% increase in fragments having sizes of 50 bases to 105 bases in the subject compared to the healthy control. In some embodiments, the cfDNA fragment size distribution shows at least a 8% increase in fragments having sizes of 50 bases to 105 bases in the subject compared to the healthy control. In some embodiments, the cfDNA fragment size distribution shows at least a 9% increase in fragments having sizes of 50 bases to 105 bases in the subject compared to the healthy control. In some embodiments, the cfDNA fragment size distribution shows at least a 10% increase in fragments having sizes of 50 bases to 105 bases in the subject compared to the healthy control. In some embodiments, the cfDNA fragment size distribution shows at least a 15% increase in fragments having sizes of 50 bases to 105 bases in the subject compared to the healthy control. In some embodiments, the cfDNA fragment size distribution shows at least a 20% increase in fragments having sizes of 50 bases to 105 bases in the subject compared to the healthy control.

In certain aspects of computer-readable media provided herein, the subject may cancer but in the very early stages, in some instances, before it can be detected using conventional methods. In some cases, the subject does not have a diagnosis of metastatic cancer. Alternatively, the subject has a low tumor burden, for example, the subject may have a tumor burden of less than 20%. In some embodiments, the subject has a tumor burden of less than 10%. In some embodiments, the subject has a tumor burden of less than 9%. In some embodiments, the subject has a tumor burden of less than 8%. In some embodiments, the subject has a tumor burden of less than 7%. In some embodiments, the subject has a tumor burden of less than 6%. In some embodiments, the subject has a tumor burden of less than 5%. Alternatively, the early stage cancers detected using methods herein comprise a non-metastatic cancer having an early stage, which is sometimes classified using a numerical classification system. In some cases, an early stage cancer is staged according to the type of cancer. In some embodiments, the non-metastatic cancer is stage 0, stage 1, stage 2, or stage 3.

Cancers and tumor cfDNA detectable using computer-readable media herein may include but are not limited to colon cancer, non-small cell lung cancer, small cell lung cancer, breast cancer, hepatocellular carcinoma, liver cancer, skin cancer, malignant melanoma, endometrial cancer, esophageal cancer, gastric cancer, ovarian cancer, pancreatic cancer, or brain cancer.

In computer-readable media provided herein, in some cases, the computer-readable media implements preparation of a report containing additional steps for addressing the cancer. In some embodiments, the report recommends a treatment for the cancer in the subject. In some embodiments, the report recommends administration of a chemotherapy to the subject. In some embodiments, the report recommends additional cancer monitoring to the subject.

A computer for use in the system can comprise one or more processors. Processors may be associated with one or more controllers, calculation units, and/or other units of a computer system, or implanted in firmware as desired. If implemented in software, the routines may be stored in any computer readable memory such as in RAM, ROM, flash memory, a magnetic disk, a laser disk, or other suitable storage medium. Likewise, this software may be delivered to a computing device via various delivery methods including, for example, over a communication channel such as a telephone line, the internet, a wireless connection, etc., or via a transportable medium, such as a computer readable disk, flash drive, etc. The various steps may be implemented as various blocks, operations, tools, modules and techniques which, in turn, may be implemented in hardware, firmware, software, or any combination of hardware, firmware, and/or software. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc. A client-server, relational database architecture can be used in embodiments of the system. A client-server architecture is a network architecture in which each computer or process on the network is either a client or a server. Server computers may be powerful computers dedicated to managing disk drives (file servers), printers (print servers), or network traffic (network servers). Client computers include PCs (personal computers) or workstations on which users run applications, as well as example output devices as disclosed herein. Client computers rely on server computers for resources, such as files, devices, and even processing power. In some embodiments, the server computer handles all of the database functionality. The client computer can have software that handles all the front-end data management and can also receive data input from users.

The system can be configured to receive a user request to perform a detection reaction on a sample. The user request may be direct or indirect. Examples of direct request include those transmitted by way of an input device, such as a keyboard, mouse, or touch screen. Examples of indirect requests include transmission via a communication medium, such as over the internet (either wired or wireless).

The system can further comprise an amplification system that performs a nucleic acid amplification reaction on the sample or a portion thereof in response to the user request. A variety of methods of amplifying polynucleotides (e.g. DNA and/or RNA) are available. Amplification may be linear, exponential, or involve both linear and exponential phases in a multi-phase amplification process. Amplification methods may involve changes in temperature, such as a heat denaturation step, or may be isothermal processes that do not require heat denaturation. Non-limiting examples of suitable amplification processes are described herein, such as with regard to any of the various aspects of the disclosure. In some embodiments, amplification comprises rolling circle amplification (RCA). A variety of systems for amplifying polynucleotides are available, and may vary based on the type of amplification reaction to be performed. For example, for amplification methods that comprise cycles of temperature changes, the amplification system may comprise a thermocycler. An amplification system can comprise a real-time amplification and detection instrument, such as systems manufactured by Applied Biosystems, Roche, and Strategene. In some embodiments, the amplification reaction comprises the steps of (i) circularizing individual polynucleotides to form a plurality of circular polynucleotides, each of which having a junction between the 5′ end and 3′ end; and (ii) amplifying the circular polynucleotides. Samples, polynucleotides, primers, polymerases, and other reagents can be any of those described herein, such as with regard to any of the various aspects. Non-limiting examples of circularization processes (e.g. with and without adapter oligonucleotides), reagents (e.g. types of adapters, use of ligases), reaction conditions (e.g. favoring self-joining), any necessary additional processing (e.g. post-reaction purification), and the junctions formed thereby are provided herein, such as with regard to any of the various aspects of the disclosure. Systems can be selected and or designed to execute any such methods.

Systems may further comprise a sequencing system that generates sequencing reads for polynucleotides amplified by the amplification system, identifies sequence differences between sequencing reads and a reference sequence, and calls a sequence difference that occurs in at least two circular polynucleotides having different junctions as the sequence variant. The sequencing system and the amplification system may be the same, or comprise overlapping equipment. For example, both the amplification system and sequencing system may utilize the same thermocycler. A variety of sequencing platforms for use in the system are available, and may be selected based on the selected sequencing method. Examples of sequencing methods are described herein. Amplification and sequencing may involve the use of liquid handlers. Several commercially available liquid handling systems can be utilized to run the automation of these processes (see for example liquid handlers from Perkin-Elmer, Beckman Coulter, Caliper Life Sciences, Tecan, Eppendorf, Apricot Design, Velocity 11 as examples). A variety of automated sequencing machines are commercially available, and include sequencers manufactured by Life Technologies (SOLiD platform, and pH-based detection), Roche (454 platform), Illumina (e.g. flow cell based systems, such as Genome Analyzer devices). Transfer between 2, 3, 4, 5, or more automated devices (e.g. between one or more of a liquid handler and a sequencing device) may be manual or automated.

The system can further comprise a report generator that sends a report to a recipient, wherein the report contains results for detection of the sequence variant. A report may be generated in real-time, such as during a sequencing read or while sequencing data is being analyzed, with periodic updates as the process progresses. In addition, or alternatively, a report may be generated at the conclusion of the analysis. The report may be generated automatically, such when the sequencing system completes the step of calling all sequence variants. In some embodiments, the report is generated in response to instructions from a user. In addition to the results of detection of the sequence variant, a report may also contain an analysis based on the one or more sequence variants. For example, where one or more sequence variants are associated with a particular contaminant or phenotype, the report may include information concerning this association, such as a likelihood that the contaminant or phenotype is present, at what level, and in some cases a suggestion based on this information (e.g. additional tests, monitoring, or remedial measures). The report can take any of a variety of forms. It is envisioned that data relating to the present disclosure can be transmitted over such networks or connections (or any other suitable approach for transmitting information, including but not limited to mailing a physical report, such as a print-out) for reception and/or for review by a receiver. The receiver can be but is not limited to an individual, or electronic system (e.g. one or more computers, and/or one or more servers).

A machine readable medium comprising computer-executable code may take many forms, including but not limited to, a tangible storage medium, a carrier wave medium or physical transmission medium. Non-volatile storage media include, for example, optical or magnetic disks, such as any of the storage devices in any computers) or the like, such as may be used to implement the databases, etc. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The subject computer-executable code can be executed on any suitable device comprising a processor, including a server, a PC, or a mobile device such as a smartphone or tablet. Any controller or computer in some cases includes a monitor, which can be a cathode ray tube (“CRT”) display, a flat panel display (e.g., active matrix liquid crystal display, liquid crystal display, etc.), or others. Computer circuitry may be placed in a box, which includes numerous integrated circuit chips, such as a microprocessor, memory, interface circuits, and others. The box also in some cases includes a hard disk drive, a floppy disk drive, a high capacity removable drive such as a writeable CD-ROM, and other common peripheral elements. Inputting devices such as a keyboard, mouse, or touch-sensitive screen, in some cases provide for input from a user. The computer can include appropriate software for receiving user instructions, either in the form of user input into a set of parameter fields, e.g., in a GUI, or in the form of preprogrammed instructions, e.g., preprogrammed for a variety of different specific operations.

### Computer Systems

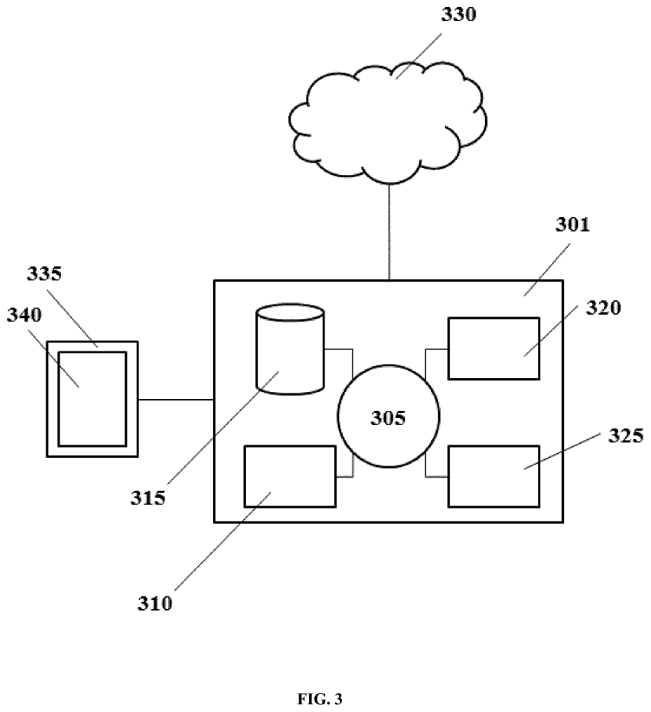

The present disclosure provides computer systems that are programmed to implement methods of the disclosure. FIG. 3 shows a computer system 301 that is programmed or otherwise configured to implement methods of the present disclosure. The computer system 301 can regulate various aspects of methods of the present disclosure, such as, for example, methods for determining that a subject has or is at risk of having a disease (e.g., cancer).

The computer system 301 includes a central processing unit (CPU, also “processor” and “computer processor” herein) 305, which can be a single core or multi core processor, or a plurality of processors for parallel processing. The computer system 301 also includes memory or memory location 310 (e.g., random-access memory, read-only memory, flash memory), electronic storage unit 315 (e.g., hard disk), communication interface 320 (e.g., network adapter) for communicating with one or more other systems, and peripheral devices 325, such as cache, other memory, data storage and/or electronic display adapters. The memory 310, storage unit 315, interface 320 and peripheral devices 325 are in communication with the CPU 305 through a communication bus (solid lines), such as a motherboard. The storage unit 315 can be a data storage unit (or data repository) for storing data. The computer system 301 can be operatively coupled to a computer network (“network”) 330 with the aid of the communication interface 320. The network 330 can be the Internet, an internet and/or extranet, or an intranet and/or extranet that is in communication with the Internet. The network 330 in some cases is a telecommunication and/or data network. The network 330 can include one or more computer servers, which can enable distributed computing, such as cloud computing. The network 330, in some cases with the aid of the computer system 301, can implement a peer-to-peer network, which may enable devices coupled to the computer system 301 to behave as a client or a server.

The CPU 305 can execute a sequence of machine-readable instructions, which can be embodied in a program or software. The instructions may be stored in a memory location, such as the memory 310. The instructions can be directed to the CPU 305, which can subsequently program or otherwise configure the CPU 305 to implement methods of the present disclosure. Examples of operations performed by the CPU 305 can include fetch, decode, execute, and writeback.

The CPU 305 can be part of a circuit, such as an integrated circuit. One or more other components of the system 301 can be included in the circuit. In some cases, the circuit is an application specific integrated circuit (ASIC).

The storage unit 315 can store files, such as drivers, libraries and saved programs. The storage unit 315 can store user data, e.g., user preferences and user programs. The computer system 301 in some cases can include one or more additional data storage units that are external to the computer system 301, such as located on a remote server that is in communication with the computer system 301 through an intranet or the Internet.

The computer system 301 can communicate with one or more remote computer systems through the network 330. For instance, the computer system 301 can communicate with a remote computer system of a user (e.g., a healthcare provider or patient). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 301 via the network 330.

Methods as described herein can be implemented by way of machine (e.g., computer processor) executable code stored on an electronic storage location of the computer system 301, such as, for example, on the memory 310 or electronic storage unit 315. The machine executable or machine readable code can be provided in the form of software. During use, the code can be executed by the processor 305. In some cases, the code can be retrieved from the storage unit 315 and stored on the memory 310 for ready access by the processor 305. In some situations, the electronic storage unit 315 can be precluded, and machine-executable instructions are stored on memory 310.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 301, can be embodied in programming Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 301 can include or be in communication with an electronic display 335 that comprises a user interface (UI) 340 for providing, for example, results of methods of the present disclosure. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 305. The algorithm can be, for example, a trained algorithm (or trained machine learning algorithm), such as, for example, a support vector machine or neural network.

### Methods of Amplification and Library Preparation

Methods herein may comprise amplification of polynucleotides present in a sample from a subject. Methods of amplification used herein may comprise rolling-circle amplification. Alternatively or in combination, methods of amplification used herein may comprise PCR. In some cases, methods of amplification herein comprise linear amplification. In some cases, amplification is not targeted to one gene or set of genes and the entire nucleic acid sample is amplified. In some cases, the method comprises circularizing individual polynucleotides of the plurality to form a plurality of circular polynucleotides, each of which having a junction between the 5′ end and the 3′ end and amplifying the circular polynucleotides of to produce amplified polynucleotides. In additional cases, methods of amplification comprise shearing the amplified polynucleotides to produce sheared polynucleotides, each sheared polynucleotide comprising one or more shear points at a 5′ end and/or 3′ end. In some cases, the method comprises enriching for a target sequence or a plurality of target sequences. In some cases, the method does not comprise enriching for a target sequence. In some cases, the method does not comprise aligning or mapping a cfDNA polynucleotide sequence to a reference genome.

In general, joining ends of a polynucleotide to one-another to form a circular polynucleotide (either directly, or with one or more intermediate adapter oligonucleotides) produces a junction having a junction sequence. Where the 5′ end and 3′ end of a polynucleotide are joined via an adapter polynucleotide, the term “junction” can refer to a junction between the polynucleotide and the adapter (e.g. one of the 5′ end junction or the 3′ end junction), or to the junction between the 5′ end and the 3′ end of the polynucleotide as formed by and including the adapter polynucleotide. Where the 5′ end and the 3′ end of a polynucleotide are joined without an intervening adapter (e.g. the 5′ end and 3′ end of a single-stranded DNA), the term “junction” refers to the point at which these two ends are joined. A junction may be identified by the sequence of nucleotides comprising the junction (also referred to as the “junction sequence”).

In some embodiments, samples comprise polynucleotides having a mixture of ends formed by natural degradation processes (such as cell lysis, cell death, and other processes by which polynucleotides such as DNA and RNA are released from a cell to its surrounding environment in which it may be further degraded, e.g., cell-free polynucleotides, e.g., cell-free DNA and cell-free RNA). Where polynucleotide ends are joined without an intervening adapter, a junction sequence may be identified by alignment to a reference sequence. For example, where the order of two component sequences appears to be reversed with respect to the reference sequence, the point at which the reversal appears to occur may be an indication of a junction at that point. Where polynucleotide ends are joined via one or more adapter sequences, a junction may be identified by proximity to the known adapter sequence, or by alignment as above if a sequencing read is of sufficient length to obtain sequence from both the 5′ and 3′ ends of the circularized polynucleotide.

In some embodiments, circularizing individual polynucleotides is effected by subjecting the plurality of polynucleotides to a ligation reaction. The ligation reaction may comprise a ligase enzyme. In some embodiments, the ligase enzyme is degraded prior to amplifying. Degradation of ligase prior to amplifying can increase the recovery rate of amplifiable polynucleotides. In some embodiments, the plurality of circularized polynucleotides is not purified or isolated prior to amplification. In some embodiments, uncircularized, linear polynucleotides are degraded prior to amplifying.

Polynucleotides (e.g., polynucleotides from a sample) may be enriched prior to circularization. This may be performed using target specific primers. Alternatively, this may be performed using capture sequences, such as pull-down probes or capture sequences attached to a substrate (e.g., pull-down probes or capture sequences attached to an array or beads). Bait sets may be used to enrich for target-specific sequences before circularization.

In some cases, circularizing in comprises the operation of joining and adapter polynucleotide to the 5′ end, the 3′ end, or both the 5′ end and the 3′ end of a polynucleotide in the plurality of polynucleotides. As previously described, where the 5′ end and/or 3′ end of a polynucleotide are joined via an adapter polynucleotide, the term “junction” can refer to the junction between the polynucleotide and the adapter (e.g., one of the 5′ end junction or the 3′ end junction), or to the junction between the 5′ end and the 3′ end of the polynucleotide as formed by and including the adapter polynucleotide.

The circularized polynucleotides can be amplified, for example, after degradation of the ligase enzyme, to yield amplified polynucleotides. Amplifying the circular polynucleotides can be effected by a polymerase. In some cases, the polymerase is a polymerase having strand-displacement activity. In some cases, the polymerase is a Phi29 DNA polymerase. Alternatively, the polymerase is a polymerase that does not have strand-displacement activity. In some cases, the polymerase is a T4 DNA polymerase or a T7 DNA polymerase. Alternately or in combination, the polymerase is a Taq polymerase, or polymerase in the Taq polymerase family. In some cases, amplification comprises rolling circle amplification (RCA). The amplified polynucleotides resulting from RCA can comprise linear concatemers, or polynucleotides comprising more than one copy of a target sequence (e.g., subunit sequence) from a template polynucleotide. In some embodiments, amplifying comprises subjecting the circular polynucleotides to an amplification reaction mixture comprising random primers. In some embodiments, amplifying comprises subjecting the circular polynucleotides to an amplification reaction mixture comprising targeted primers. Alternatively, the circular polynucleotides may be amplified in an untargeted manner and enriched for one or more target sequences after amplification. In some cases, amplifying comprises subjecting the circular polynucleotides to an amplification reaction mixture comprising one or more primers, each of which specifically hybridizes to a different target sequence via sequence complementarity. In some cases, amplifying comprises subjecting the circular polynucleotides to an amplification reaction mixture comprising inverse primers.

The amplified polynucleotides are sheared, in some cases, to produce sheared polynucleotides that are shorter in length relative to the unsheared polynucleotides. Two or more sheared polynucleotides originating from the same linear concatemer may have the same junction sequence but can have different 5′ and/or 3′ ends (e.g., shear ends).

Amplified polynucleotides can be sheared using any variety of methods, such as, but not limited to, physical fragmentation, enzymatic methods, and chemical fragmentation. Non-limiting examples of physical fragmentation methods that can be employed for the fragmentation of amplified polynucleotides include acoustic shearing, sonication, and hydrodynamic shearing. In some cases, acoustic shearing and sonication may be used. Non-limiting examples of enzymatic fragmentation methods that can be employed for the fragmentation of amplified polynucleotides include use of enzymes such as DNase I and other restriction endonucleases, including non-specific nucleases, and transposases. Non-limiting examples of chemical fragmentation methods that can be employed for the fragmentation of amplified polynucleotides include use of heat and divalent metal cations.

Sheared polynucleotides (also referred to as fragmented polynucleotides) which are shorter in length compared to the unsheared polynucleotides may be desired to match the capabilities of the sequencing instrument used for producing sequencing reads, also referred to as sequence reads. For example, amplified polynucleotides may be fragmented, for example sheared, to the optimal length determined by the downstream sequencing platform. Various sequencing instruments, further described herein, can accommodate nucleic acids of different lengths. In some cases, amplified polynucleotides are sheared in the process of attaching adapters useful in downstream sequencing platforms, for example in flow cell attachment or sequencing primer binding. In some cases, sheared polynucleotides are subject to amplification to produce amplification products of the sheared polynucleotides prior to sequencing. Additional amplification can be desirable, for example, to generate a sufficient amount of polynucleotides for downstream analysis, for example, sequencing analysis. The resulting amplification products can comprise multiple copies of individual sheared polynucleotides.

Cell-free polynucleotides from a sample may be any of a variety of polynucleotides, including but not limited to, DNA, RNA, ribosomal RNA (rRNA), transfer RNA (tRNA), micro RNA (miRNA), messenger RNA (mRNA), fragments of any of these, or combinations of any two or more of these. In some embodiments, samples comprise DNA. In some embodiments, samples comprise cell-free genomic DNA. In some embodiments, the samples comprise DNA generated by amplification, such as by primer extension reactions using any suitable combination of primers and a DNA polymerase, including but not limited to polymerase chain reaction (PCR), reverse transcription, and combinations thereof. Where the template for the primer extension reaction is RNA, the product of reverse transcription is referred to as complementary DNA (cDNA). Primers useful in primer extension reactions can comprise sequences specific to one or more targets, random sequences, partially random sequences, and combinations thereof. In general, sample polynucleotides comprise any polynucleotide present in a sample, which may or may not include target polynucleotides. The polynucleotides may be single-stranded, double-stranded, or a combination of these. In some embodiments, polynucleotides subjected to a method of the disclosure are single-stranded polynucleotides, which may or may not be in the presence of double-stranded polynucleotides. In some embodiments, the polynucleotides are single-stranded DNA. Single-stranded DNA (ssDNA) may be ssDNA that is isolated in a single-stranded form, or DNA that is isolated in double-stranded form and subsequently made single-stranded for the purpose of one or more steps in a method of the disclosure.

In some embodiments, polynucleotides are subjected to subsequent steps (e.g. circularization and amplification) without an extraction step, and/or without a purification step. For example, a fluid sample may be treated to remove cells without an extraction step to produce a purified liquid sample and a cell sample, followed by isolation of DNA from the purified fluid sample. A variety of procedures for isolation of polynucleotides are available, such as by precipitation or non-specific binding to a substrate followed by washing the substrate to release bound polynucleotides. Where polynucleotides are isolated from a sample without a cellular extraction step, polynucleotides will largely be extracellular or “cell-free” polynucleotides, such as cell-free DNA and cell-free RNA, which may correspond to dead or damaged cells. The identity of such cells may be used to characterize the cells or population of cells from which they are derived, such as tumor cells (e.g. in cancer detection), fetal cells (e.g. in prenatal diagnostic), cells from transplanted tissue (e.g. in early detection of transplant failure), or members of a microbial community.

If a sample is treated to extract polynucleotides, such as from cells in a sample, a variety of extraction methods are available. For example, nucleic acids can be purified by organic extraction with phenol, phenol/chloroform/isoamyl alcohol, or similar formulations, including TRIzol and TriReagent. Other non-limiting examples of extraction techniques include: (1) organic extraction followed by ethanol precipitation, e.g., using a phenol/chloroform organic reagent (Ausubel et al., 1993, which is entirely incorporated herein by reference), with or without the use of an automated nucleic acid extractor, e.g., the Model 341 DNA Extractor available from Applied Biosystems (Foster City, Calif.); (2) stationary phase adsorption methods (U.S. Pat. No. 5,234,809; Walsh et al., 1991, each of which is entirely incorporated herein by reference); and (3) salt-induced nucleic acid precipitation methods (Miller et al., (1988), which is entirely incorporated herein by reference), such precipitation methods may be referred to as “salting-out” methods. Another example of nucleic acid isolation and/or purification includes the use of magnetic particles to which nucleic acids can specifically or non-specifically bind, followed by isolation of the beads using a magnet, and washing and eluting the nucleic acids from the beads (see e.g. U.S. Pat. No. 5,705,628, which is entirely incorporated herein by reference). In some embodiments, the above isolation methods may be preceded by an enzyme digestion step to help eliminate unwanted protein from the sample, e.g., digestion with proteinase K, or other like proteases. See, e.g., U.S. Pat. No. 7,001,724, which is entirely incorporated herein by reference. If desired, RNase inhibitors may be added to the lysis buffer. For certain cell or sample types, it may be desirable to add a protein denaturation/digestion step to the protocol. Purification methods may be directed to isolate DNA, RNA, or both. When both DNA and RNA are isolated together during or subsequent to an extraction procedure, further steps may be employed to purify one or both separately from the other. Sub-fractions of extracted nucleic acids can also be generated, for example, purification by size, sequence, or other physical or chemical characteristic. In addition to an initial nucleic acid isolation step, purification of nucleic acids can be performed after any step in the disclosed methods, such as to remove excess or unwanted reagents, reactants, or products. A variety of methods for determining the amount and/or purity of nucleic acids in a sample are available, such as by absorbance (e.g. absorbance of light at 260 nm, 280 nm, and a ratio of these) and detection of a label (e.g. fluorescent dyes and intercalating agents, such as SYBR green, SYBR blue, DAPI, propidium iodine, Hoechst stain, SYBR gold, ethidium bromide).

Where desired, polynucleotides from a sample may be fragmented prior to further processing. Fragmentation may be accomplished by any of a variety of methods, including chemical, enzymatic, and mechanical fragmentation. In some embodiments, the fragments have an average or median length from about 10 to about 1,000 nucleotides in length, such as between 10-800, 10-500, 50-500, 90-200, or 50-150 nucleotides. In some embodiments, the fragments have an average or median length of about or less than about 100, 200, 300, 500, 600, 800, 1000, or 1500 nucleotides. In some embodiments, the fragments range from about 90-200 nucleotides, and/or have an average length of about 150 nucleotides. In some embodiments, the fragmentation is accomplished mechanically comprising subjecting sample polynucleotides to acoustic sonication. In some embodiments, the fragmentation comprises treating the sample polynucleotides with one or more enzymes under conditions suitable for the one or more enzymes to generate double-stranded nucleic acid breaks. Examples of enzymes useful in the generation of polynucleotide fragments include sequence specific and non-sequence specific nucleases. Non-limiting examples of nucleases include DNase I, Fragmentase, restriction endonucleases, variants thereof, and combinations thereof. For example, digestion with DNase I can induce random double-stranded breaks in DNA in the absence of Mg++ and in the presence of Mn++. In some embodiments, fragmentation comprises treating the sample polynucleotides with one or more restriction endonucleases. Fragmentation can produce fragments having 5′ overhangs, 3′ overhangs, blunt ends, or a combination thereof. In some embodiments, such as when fragmentation comprises the use of one or more restriction endonucleases, cleavage of sample polynucleotides leaves overhangs having a predictable sequence. Fragmented polynucleotides may be subjected to a step of size selecting the fragments via standard methods such as column purification, bead purification, or isolation from an agarose gel.

In some cases, methods herein comprise digesting polynucleotides, including DNA and cfDNA with a nuclease, such as a DNase, that cleaves DNA including cfDNA that is free of DNA-binding proteins. Some such methods provide information for mapping protein binding sites on DNA including cfDNA. In these methods, DNA including cfDNA is isolated to preserve DNA-protein interactions and then treated with a DNase, such as DNase I to cleave DNA including cfDNA at the protein free region of the DNA fragments. Cleaved DNA including cfDNA is further purified to remove protein using any DNA extraction methods provided herein and then used in any library preparation methods provided herein including but not limited to circularization, single stranded DNA library preparation, and double stranded DNA library preparation. In some cases DNase I treatment of DNA comprises isolation of DNA, treating the DNA with DNase I, removing protein from the treated DNA with a buffer, treating the DNA with T4 DNA polymerase to create blunt ends, purification of DNA using phenol extraction and ethanol precipitation, ligating linkers to the DNA prior to library preparation. In some cases, the method further comprises digesting the isolated biotinylated DNA with a restriction enzyme resulting in only the border of the DNase hypersensitive site prior to library preparation and sequencing, as described in Crawford et al. Genome Res. 2006. 16(1) 123-31, which is entirely incorporated herein by reference.

In some cases, methods herein comprise preparation of a DNA library from polynucleotides. For example, methods herein comprise preparation of a single stranded DNA library. Any suitable method of preparing a single stranded DNA library is contemplated for use in methods herein. For example, the method of preparing a single stranded DNA library comprises denaturing the DNA sample to create a plurality of ssDNA; ligating an adapter to the 3′ end of the ssDNA molecules; synthesizing a second strand using a primer complementary to the adapter; ligating a double stranded adapter to the extension products; amplifying the second strand using primers targeting the first and second adapters (for example, using PCR); and sequencing the library on a sequencer. An additional method of single stranded library preparation comprises denaturing the DNA sample to create a plurality of ssDNA; ligating an adapter to the 3′ end of the ssDNA molecules; synthesizing the second strand by using a primer complementary to the adapter; ligating a double stranded adapter to the extension products; amplifying the second strand (for example, by PCR) the second strand using primers targeting the first and second adapters; in some cases enriching for the regions of interest using hybridization with capture probes; amplifying (for example, by PCR) the captured products; and sequencing the library on a sequencer.

Further examples of single stranded library preparation include a method comprising the steps of treating the DNA with a heat labile phosphatase to remove residual phosphate groups from the 5′ and 3′ ends of the DNA strands; removal of deoxyuracils derived from cytosine deamination from the DNA strands; ligation of a 5′-phosphorylated adapter oligonucleotide having about 10 nucleotides and a long 3′ biotinylated spacer arm to the 3′ ends of the DNA strands; immobilization of adapter-ligated molecules on streptavidin beads; copying the template strand using a 5′-tailed primer complementary to the adapter using Bst polymerase; washing away excess primers; removal of 3′ overhangs using T4 DNA polymerase; joining a second adapter to the newly synthesized strands using blunt-end ligation; washing away excess adapter; releasing library molecules by heat denaturation; adding full-length adapter sequences including bar codes through amplification using tailed primers; and sequencing the library, as described in Gansauge et al. 2013. Nature Protocols. 8(4) 737-748, which is entirely incorporated herein by reference.

In additional embodiments, methods herein comprise preparation of a double stranded DNA library. Any suitable method of preparing a double stranded DNA library is contemplated for use in methods herein. For example, the method of preparing a double stranded DNA library comprises ligating sequencing adapters to the 5′ and 3′ ends of a plurality of DNA fragments and sequencing the library on a sequencer. An additional method of double stranded DNA library preparation comprises ligating adapters to the 5′ and 3′ ends of a plurality of DNA fragments; attaching the full adapter sequences to the ligated fragments through PCR using primers that are complementary to the ligated adapters; and sequencing the library on a sequencer. A further method comprises ligating adapters to the 5′ and 3′ ends of a plurality of DNA fragments; amplifying the ligated product through PCR that are complementary to the ligated adapters; in some cases enriching for the regions of interest through hybridization with capture probes; PCR amplifying the captured products; and sequencing the library on a sequencer. An additional method of double stranded library preparation comprises ligating adapters to the 5′ and 3′ ends of a plurality of DNA fragments; amplifying the ligated product through PCR using primers that are complementary to the ligated adapters; circularizing the double stranded PCR products or denature and circularize the single stranded PCR products; in some cases enriching for the regions of interest by PCR using primers targeting specific genes; and sequencing the library on a sequencer.

Further examples of double stranded library preparation include the Safe-Sequencing System described in Kinde et al. (Kinde et al. 2011. Proc. Natl. Acad. Sci., USA, 108(23) 9530-9535, which is entirely incorporated herein by reference) which comprises assignment of a unique identifier (UID) to each template molecule; amplification of each uniquely tagged template molecule to create UID families; and redundant sequencing of the amplification products. An additional example comprises the circulating single-molecule amplification and resequencing technology (cSMART) described in Lv et al. (Lv et al. 2015. Clin. Chem., 61(1) 172-181, which is entirely incorporated herein by reference) which tags single molecules with unique barcodes, circularizes, targets alleles for replication by inverse PCR, then sequencing the prepared library and counts the alleles present.

In some library preparation approaches provided herein, certain nucleic acid molecules (e.g., cfDNA polynucleotides) are selected or enriched from a plurality of nucleic acid molecules (e.g., total cfDNA). Certain nucleic acid molecules or target sequences may be selected or enriched when they are more likely to result in informative results. For example, certain nucleic acid molecules or target sequences may be selected when they correspond to cfDNA sequences having altered size differences in subjects who have cancer (e.g., early stage cancer) as compared to healthy subjects. Certain nucleic acid molecules may be selected or enriched by amplification with target specific primers. Certain nucleic acid molecules may be selected or enriched by binding target nucleic acid molecules to probes. For example, such nucleic acid molecules are selected or enriched using bait sets.

In additional library preparation methods, cfDNA fragments having certain features are selected using an antibody. In some cases, cfDNA fragments that are methylated or hypermethylated are selected using an antibody. Selected cfDNA fragments are then used in any library preparation method described herein, including circularization, single stranded DNA library preparation, and double stranded DNA library preparation. Sequencing such isolated cfDNA fragments provides information as to the features present in the cfDNA, including modifications such as methylation or hypermethylation.

According to some embodiments, polynucleotides among the plurality of polynucleotides from a sample are circularized. Circularization can include joining the 5′ end of a polynucleotide to the 3′ end of the same polynucleotide, to the 3′ end of another polynucleotide in the sample, or to the 3′ end of a polynucleotide from a different source (e.g. an artificial polynucleotide, such as an oligonucleotide adapter). In some embodiments, the 5′ end of a polynucleotide is joined to the 3′ end of the same polynucleotide (also referred to as “self-joining”). In some embodiment, conditions of the circularization reaction are selected to favor self-joining of polynucleotides within a particular range of lengths, so as to produce a population of circularized polynucleotides of a particular average length. For example, circularization reaction conditions may be selected to favor self-joining of polynucleotides shorter than about 5000, 2500, 1000, 750, 500, 400, 300, 200, 150, 100, 50, or fewer nucleotides in length. In some embodiments, fragments having lengths between 50-5000 nucleotides, 100-2500 nucleotides, or 150-500 nucleotides are favored, such that the average length of circularized polynucleotides falls within the respective range. In some embodiments, 80% or more of the circularized fragments are between 50-500 nucleotides in length, such as between 50-200 nucleotides in length. Reaction conditions that may be optimized include the length of time allotted for a joining reaction, the concentration of various reagents, and the concentration of polynucleotides to be joined. In some embodiments, a circularization reaction preserves the distribution of fragment lengths present in a sample prior to circularization. For example, one or more of the mean, median, mode, and standard deviation of fragment lengths in a sample before circularization and of circularized polynucleotides are within 75%, 80%, 85%, 90%, 95%, or more of one another.

In some cases, rather than preferentially forming self-joining circularization products, one or more adapter oligonucleotides are used, such that the 5′ end and 3′ end of a polynucleotide in the sample are joined by way of one or more intervening adapter oligonucleotides to form a circular polynucleotide. For example, the 5′ end of a polynucleotide can be joined to the 3′ end of an adapter, and the 5′ end of the same adapter can be joined to the 3′ end of the same polynucleotide. An adapter oligonucleotide includes any oligonucleotide having a sequence, at least a portion of which is known, that can be joined to a sample polynucleotide. Adapter oligonucleotides can comprise DNA, RNA, nucleotide analogues, non-canonical nucleotides, labeled nucleotides, modified nucleotides, or combinations thereof. Adapter oligonucleotides can be single-stranded, double-stranded, or partial duplex. In general, a partial-duplex adapter comprises one or more single-stranded regions and one or more double-stranded regions. Double-stranded adapters can comprise two separate oligonucleotides hybridized to one another (also referred to as an “oligonucleotide duplex”), and hybridization may leave one or more blunt ends, one or more 3′ overhangs, one or more 5′ overhangs, one or more bulges resulting from mismatched and/or unpaired nucleotides, or any combination of these. When two hybridized regions of an adapter are separated from one another by a non-hybridized region, a “bubble” structure results. Adapters of different kinds can be used in combination, such as adapters of different sequences. Different adapters can be joined to sample polynucleotides in sequential reactions or simultaneously. In some embodiments, identical adapters are added to both ends of a target polynucleotide. For example, first and second adapters can be added to the same reaction. Adapters can be manipulated prior to combining with sample polynucleotides. For example, terminal phosphates can be added or removed.

Where adapter oligonucleotides are used, the adapter oligonucleotides can contain one or more of a variety of sequence elements, including but not limited to, one or more amplification primer annealing sequences or complements thereof, one or more sequencing primer annealing sequences or complements thereof, one or more barcode sequences, one or more common sequences shared among multiple different adapters or subsets of different adapters, one or more restriction enzyme recognition sites, one or more overhangs complementary to one or more target polynucleotide overhangs, one or more probe binding sites (e.g. for attachment to a sequencing platform, such as a flow cell for massive parallel sequencing, such as flow cells as developed by Illumina, Inc.), one or more random or near-random sequences (e.g. one or more nucleotides selected at random from a set of two or more different nucleotides at one or more positions, with each of the different nucleotides selected at one or more positions represented in a pool of adapters comprising the random sequence), and combinations thereof. In some cases, the adapters may be used to purify those circles that contain the adapters, for example by using beads (particularly magnetic beads for ease of handling) that are coated with oligonucleotides comprising a complementary sequence to the adapter, that can “capture” the closed circles with the correct adapters by hybridization thereto, wash away those circles that do not contain the adapters and any unligated components, and then release the captured circles from the beads. In addition, in some cases, the complex of the hybridized capture probe and the target circle can be directly used to generate concatemers, such as by direct rolling circle amplification (RCA). In some embodiments, the adapters in the circles can also be used as a sequencing primer. Two or more sequence elements can be non-adjacent to one another (e.g. separated by one or more nucleotides), adjacent to one another, partially overlapping, or completely overlapping. For example, an amplification primer annealing sequence can also serve as a sequencing primer annealing sequence. Sequence elements can be located at or near the 3′ end, at or near the 5′ end, or in the interior of the adapter oligonucleotide. A sequence element may be of any suitable length, such as about or less than about 3, 4, 5, 6, 7, 8, 9, 10, 15, 20, 25, 30, 35, 40, 45, 50 or more nucleotides in length. Adapter oligonucleotides can have any suitable length, at least sufficient to accommodate the one or more sequence elements of which they are comprised. In some embodiments, adapters are about or less than about 10, 15, 20, 25, 30, 35, 40, 45, 50, 55, 60, 65, 70, 75, 80, 90, 100, 200, or more nucleotides in length. In some embodiments, an adapter oligonucleotide is in the range of about 12 to 40 nucleotides in length, such as about 15 to 35 nucleotides in length.

In some embodiments, the adapter oligonucleotides joined to fragmented polynucleotides from one sample comprise one or more sequences common to all adapter oligonucleotides and a barcode that is unique to the adapters joined to polynucleotides of that particular sample, such that the barcode sequence can be used to distinguish polynucleotides originating from one sample or adapter joining reaction from polynucleotides originating from another sample or adapter joining reaction. In some embodiments, an adapter oligonucleotide comprises a 5′ overhang, a 3′ overhang, or both that is complementary to one or more target polynucleotide overhangs. Complementary overhangs can be one or more nucleotides in length, including but not limited to 1, 2, 3, 4, 5, 6, 7, 8, 9, 10, 11, 12, 13, 14, 15, or more nucleotides in length. Complementary overhangs may comprise a fixed sequence. Complementary overhangs of an adapter oligonucleotide may comprise a random sequence of one or more nucleotides, such that one or more nucleotides are selected at random from a set of two or more different nucleotides at one or more positions, with each of the different nucleotides selected at one or more positions represented in a pool of adapters with complementary overhangs comprising the random sequence. In some embodiments, an adapter overhang is complementary to a target polynucleotide overhang produced by restriction endonuclease digestion. In some embodiments, an adapter overhang consists of an adenine or a thymine.

A variety of methods for circularizing polynucleotides are available. In some embodiments, circularization comprises an enzymatic reaction, such as use of a ligase (e.g. an RNA or DNA ligase). A variety of ligases are available, including, but not limited to, Circligase™ (Epicentre; Madison, Wis.), RNA ligase, T4 RNA Ligase 1 (ssRNA Ligase, which works on both DNA and RNA). In addition, T4 DNA ligase can also ligate ssDNA if no dsDNA templates are present, although this is generally a slow reaction. Other non-limiting examples of ligases include NAD-dependent ligases including Taq DNA ligase, Thermus filiformis DNA ligase, Escherichia coli DNA ligase, Tth DNA ligase, Thermus scotoductus DNA ligase (I and II), thermostable ligase, Ampligase thermostable DNA ligase, VanC-type ligase, 9° N DNA Ligase, Tsp DNA ligase, and novel ligases discovered by bioprospecting; ATP-dependent ligases including T4 RNA ligase, T4 DNA ligase, T3 DNA ligase, T7 DNA ligase, Pfu DNA ligase, DNA ligase 1, DNA ligase III, DNA ligase IV, and novel ligases discovered by bioprospecting; and wild-type, mutant isoforms, and genetically engineered variants thereof. Where self-joining is desired, the concentration of polynucleotides and enzyme can be adjusted to facilitate the formation of intramolecular circles rather than intermolecular structures. Reaction temperatures and times can be adjusted as well. In some embodiments, 60° C. is used to facilitate intramolecular circles. In some embodiments, reaction times are between 12-16 hours. Reaction conditions may be those specified by the manufacturer of the selected enzyme. In some embodiments, an exonuclease step can be included to digest any unligated nucleic acids after the circularization reaction. That is, closed circles do not contain a free 5′ or 3′ end, and thus the introduction of a 5′ or 3′ exonuclease will not digest the closed circles but will digest the unligated components. This may find particular use in multiplex systems.

In general, joining ends of a polynucleotide to one-another to form a circular polynucleotide (either directly, or with one or more intermediate adapter oligonucleotides) produces a junction having a junction sequence. Where the 5′ end and 3′ end of a polynucleotide are joined via an adapter polynucleotide, the term “junction” can refer to a junction between the polynucleotide and the adapter (e.g. one of the 5′ end junction or the 3′ end junction), or to the junction between the 5′ end and the 3′ end of the polynucleotide as formed by and including the adapter polynucleotide. Where the 5′ end and the 3′ end of a polynucleotide are joined without an intervening adapter (e.g. the 5′ end and 3′ end of a single-stranded DNA), the term “junction” refers to the point at which these two ends are joined. A junction may be identified by the sequence of nucleotides comprising the junction (also referred to as the “junction sequence”). In some embodiments, samples comprise polynucleotides having a mixture of ends formed by natural degradation processes (such as cell lysis, cell death, and other processes by which DNA is released from a cell to its surrounding environment in which it may be further degraded, such as in cell-free polynucleotides, such as cell-free DNA and cell-free RNA), fragmentation that is a byproduct of sample processing (such as fixing, staining, and/or storage procedures), and fragmentation by methods that cleave DNA without restriction to specific target sequences (e.g. mechanical fragmentation, such as by sonication; non-sequence specific nuclease treatment, such as DNase I, fragmentase). Where samples comprise polynucleotides having a mixture of ends, the likelihood that two polynucleotides will have the same 5′ end or 3′ end is low, and the likelihood that two polynucleotides will independently have both the same 5′ end and 3′ end is extremely low. Accordingly, in some embodiments, junctions may be used to distinguish different polynucleotides, even where the two polynucleotides comprise a portion having the same target sequence. Where polynucleotide ends are joined without an intervening adapter, a junction sequence may be identified by alignment to a reference sequence. For example, where the order of two component sequences appears to be reversed with respect to the reference sequence, the point at which the reversal appears to occur may be an indication of a junction at that point. Where polynucleotide ends are joined via one or more adapter sequences, a junction may be identified by proximity to the known adapter sequence, or by alignment as above if a sequencing read is of sufficient length to obtain sequence from both the 5′ and 3′ ends of the circularized polynucleotide. In some embodiments, the formation of a particular junction is a sufficiently rare event such that it is unique among the circularized polynucleotides of a sample.

### Methods of Sequencing

According to some embodiments, linear and/or circularized polynucleotides (or amplification products thereof, which may have been enriched in some cases) are subjected to a sequencing reaction to generate sequencing reads. Sequencing reads produced by such methods may be used in accordance with other methods disclosed herein. A variety of sequencing methodologies are available, particularly high-throughput sequencing methodologies. Examples include, without limitation, sequencing systems manufactured by Illumina (sequencing systems such as HiSeq® and MiSeq®), Life Technologies (Ion Torrent®, SOLiD®, etc.), Roche's 454 Life Sciences systems, Pacific Biosciences systems, etc. In some embodiments, sequencing comprises use of HiSeq® and MiSeq® systems to produce reads of about or more than about 50, 75, 100, 125, 150, 175, 200, 250, 300, or more nucleotides in length. In some embodiments, sequencing comprises a sequencing by synthesis process, where individual nucleotides are identified iteratively, as they are added to the growing primer extension product. Pyrosequencing is an example of a sequence by synthesis process that identifies the incorporation of a nucleotide by assaying the resulting synthesis mixture for the presence of by-products of the sequencing reaction, namely pyrophosphate. In particular, a primer/template/polymerase complex is contacted with a single type of nucleotide. If that nucleotide is incorporated, the polymerization reaction cleaves the nucleoside triphosphate between the α and β phosphates of the triphosphate chain, releasing pyrophosphate. The presence of released pyrophosphate is then identified using a chemiluminescent enzyme reporter system that converts the pyrophosphate, with AMP, into ATP, then measures ATP using a luciferase enzyme to produce measurable light signals. Where light is detected, the base is incorporated, where no light is detected, the base is not incorporated. Following appropriate washing steps, the various bases are cyclically contacted with the complex to sequentially identify subsequent bases in the template sequence. See, e.g., U.S. Pat. No. 6,210,891, which is entirely incorporated herein by reference.

In related sequencing processes, the primer/template/polymerase complex is immobilized upon a substrate and the complex is contacted with labeled nucleotides. The immobilization of the complex may be through the primer sequence, the template sequence and/or the polymerase enzyme, and may be covalent or noncovalent. For example, immobilization of the complex can be via a linkage between the polymerase or the primer and the substrate surface. In alternate configurations, the nucleotides are provided with and without removable terminator groups. Upon incorporation, the label is coupled with the complex and is thus detectable. In the case of terminator bearing nucleotides, all four different nucleotides, bearing individually identifiable labels, are contacted with the complex. Incorporation of the labeled nucleotide arrests extension, by virtue of the presence of the terminator, and adds the label to the complex, allowing identification of the incorporated nucleotide. The label and terminator are then removed from the incorporated nucleotide, and following appropriate washing steps, the process is repeated. In the case of non-terminated nucleotides, a single type of labeled nucleotide is added to the complex to determine whether it will be incorporated, as with pyrosequencing. Following removal of the label group on the nucleotide and appropriate washing steps, the various different nucleotides are cycled through the reaction mixture in the same process. See, e.g., U.S. Pat. No. 6,833,246, incorporated herein by reference in its entirety for all purposes. For example, the Illumina Genome Analyzer System is based on technology described in WO 98/44151, wherein DNA molecules are bound to a sequencing platform (flow cell) via an anchor probe binding site (otherwise referred to as a flow cell binding site) and amplified in situ on a glass slide. A solid surface on which DNA molecules are amplified may comprise a plurality of first and second bound oligonucleotides, the first complementary to a sequence near or at one end of a target polynucleotide and the second complementary to a sequence near or at the other end of a target polynucleotide. This arrangement permits bridge amplification, such as described in US20140121116. The DNA molecules are then annealed to a sequencing primer and sequenced in parallel base-by-base using a reversible terminator approach. Hybridization of a sequencing primer may be preceded by cleavage of one strand of a double-stranded bridge polynucleotide at a cleavage site in one of the bound oligonucleotides anchoring the bridge, thus leaving one single strand not bound to the solid substrate that may be removed by denaturing, and the other strand bound and available for hybridization to a sequencing primer. In some cases, the Illumina Genome Analyzer System utilizes flow-cells with 8 channels, generating sequencing reads of 18 to 36 bases in length, generating >1.3 Gbp of high quality data per run (see www.illumina.com).

In yet a further sequence by synthesis process, the incorporation of differently labeled nucleotides is observed in real time as template dependent synthesis is carried out. In particular, an individual immobilized primer/template/polymerase complex is observed as fluorescently labeled nucleotides are incorporated, permitting real time identification of each added base as it is added. In this process, label groups are attached to a portion of the nucleotide that is cleaved during incorporation. For example, by attaching the label group to a portion of the phosphate chain removed during incorporation, i.e., a β,γ, or other terminal phosphate group on a nucleoside polyphosphate, the label is not incorporated into the nascent strand, and instead, natural DNA is produced. Observation of individual molecules may involve the optical confinement of the complex within a very small illumination volume. By optically confining the complex, one creates a monitored region in which randomly diffusing nucleotides are present for a very short period of time, while incorporated nucleotides are retained within the observation volume for longer as they are being incorporated. This results in a characteristic signal associated with the incorporation event, which is also characterized by a signal profile that is characteristic of the base being added. In related aspects, interacting label components, such as fluorescent resonant energy transfer (FRET) dye pairs, are provided upon the polymerase or other portion of the complex and the incorporating nucleotide, such that the incorporation event puts the labeling components in interactive proximity, and a characteristic signal results, that is again, also characteristic of the base being incorporated (See, e.g., U.S. Pat. Nos. 6,917,726, 7,033,764, 7,052,847, 7,056,676, 7,170,050, 7,361,466, and 7,416,844; and US 20070134128, each of which is entirely incorporated herein by reference).

In some embodiments, the nucleic acids in the sample can be sequenced by ligation. This method may use a DNA ligase enzyme to identify the target sequence, for example, as used in the polony method and in the SOLiD technology (Applied Biosystems, now Invitrogen). In general, a pool of all possible oligonucleotides of a fixed length is provided, labeled according to the sequenced position. Oligonucleotides are annealed and ligated; the preferential ligation by DNA ligase for matching sequences results in a signal corresponding to the complementary sequence at that position.

Sequencing methods herein provide information useful in methods herein. In some cases, sequencing provides a sequence of a polymorphic region. Additionally, sequencing provides a length of a polynucleotide, such as a DNA including cfDNA. Further, sequencing provides a sequence of a breakpoint or end of a DNA such as a cfDNA. Sequencing further provides a sequence of a border of a protein binding site or a border of a DNase hypersensitive site.

### Samples

In embodiments of the various methods described herein, the sample may be from a subject. A subject may be any animal, including but not limited to, a cow, a pig, a mouse, a rat, a chicken, a cat, a dog, etc., and is usually a mammal, such as a human. Sample polynucleotides may be isolated from a subject, such as a tissue sample, bodily fluid sample, or organ sample, including, for example, biopsy, blood sample, or fluid sample containing nucleic acids (e.g. saliva). In some cases, the sample does not comprise intact cells, is treated to remove cells, or polynucleotides are isolated without a cellular extractions step (e.g. to isolate cell-free polynucleotides, such as cell-free DNA). Other examples of sample sources include those from blood, urine, feces, nares, the lungs, the gut, other bodily fluids or excretions, materials derived therefrom, or combinations thereof. In some embodiments, the sample is a blood sample or a portion thereof (e.g. blood plasma or serum). Serum and plasma may be of particular interest, due to the relative enrichment for tumor DNA associated with the higher rate of malignant cell death among such tissues. In some embodiments, a sample from a single individual is divided into multiple separate samples (e.g. 2, 3, 4, 5, 6, 7, 8, 9, 10, or more separate samples) that are subjected to methods of the disclosure independently, such as analysis in duplicate, triplicate, quadruplicate, or more. Where a sample is from a subject, the reference sequence may also be derived of the subject, such as a consensus sequence from the sample under analysis or the sequence of polynucleotides from another sample or tissue of the same subject. For example, a blood sample may be analyzed for ctDNA mutations, while cellular DNA from another sample (e.g. buccal or skin sample) is analyzed to determine the reference sequence.

Polynucleotides may be extracted from a sample according to any suitable method. A variety of kits are available for extraction of polynucleotides, selection of which may depend on the type of sample, or the type of nucleic acid to be isolated. Examples of extraction methods are provided herein, such as those described with respect to any of the various aspects disclosed herein. In one example, the sample may be a blood sample, such as a sample collected in an EDTA tube (e.g. BD Vacutainer). Plasma can be separated from the peripheral blood cells by centrifugation (e.g. 10 minutes at 1900×g at 4° C.). Plasma separation performed in this way on a 6 mL blood sample may yield 2.5 to 3 mL of plasma. Circulating cell-free DNA can be extracted from a plasma sample, such as by using a QIAmp Circulating Nucleic Acid Kit (Qiagene), according the manufacturer's protocol. DNA may then be quantified (e.g. on an Agilent 2100 Bioanalyzer with High Sensitivity DNA kit (Agilent)). As an example, yield of circulating DNA from such a plasma sample from a healthy person may range from 1 ng to 10 ng per mL of plasma, with significantly more in cancer patient samples.

In some embodiments, the plurality of polynucleotides comprises cell-free polynucleotides, such as cell-free DNA (cfDNA), cell-free RNA (cfRNA), circulating tumor DNA (ctDNA), or circulating tumor RNA (ctRNA). Cell-free DNA circulates in both healthy and diseased individuals. Cell-free RNA circulates in both healthy and diseased individuals. cfDNA from tumors (ctDNA) is not confined to any specific cancer type, but appears to be a common finding across different malignancies. According to some measurements, the free circulating DNA concentration in plasma is about 14-18 ng/ml in control subjects and about 180-318 ng/ml in patients with neoplasias. Apoptotic and necrotic cell death contribute to cell-free circulating DNA in bodily fluids. For example, significantly increased circulating DNA levels have been observed in plasma of prostate cancer patients and other prostate diseases, such as Benign Prostate Hyperplasia and Prostatits. In addition, circulating tumor DNA is present in fluids originating from the organs where the primary tumor occurs. Thus, breast cancer detection can be achieved in ductal lavages; colorectal cancer detection in stool; lung cancer detection in sputum, and prostate cancer detection in urine or ejaculate. Cell-free DNA may be obtained from a variety of sources. One common source is blood samples of a subject. However, cfDNA or other fragmented DNA may be derived from a variety of other sources. For example, urine and stool samples can be a source of cfDNA, including ctDNA. Cell-free RNA may be obtained from a variety of sources.

In some embodiments, polynucleotides are subjected to subsequent steps (e.g. circularization and amplification) without an extraction step, and/or without a purification step. For example, a fluid sample may be treated to remove cells without an extraction step to produce a purified liquid sample and a cell sample, followed by isolation of DNA from the purified fluid sample. A variety of procedures for isolation of polynucleotides are available, such as by precipitation or non-specific binding to a substrate followed by washing the substrate to release bound polynucleotides. Where polynucleotides are isolated from a sample without a cellular extraction step, polynucleotides will largely be extracellular or “cell-free” polynucleotides. For example, cell-free polynucleotides may include cell-free DNA (also called “circulating” DNA). In some embodiments, the circulating DNA is circulating tumor DNA (ctDNA) from tumor cells, such as from a body fluid or excretion (e.g. blood sample). Cell-free polynucleotides may include cell-free RNA (also called “circulating” RNA). In some embodiments, the circulating RNA is circulating tumor RNA (ctRNA) from tumor cells. Tumors frequently show apoptosis or necrosis, such that tumor nucleic acids are released into the body, including the blood stream of a subject, through a variety of mechanisms, in different forms and at different levels. In some cases, the size of the ctDNA can range between higher concentrations of smaller fragments, generally 70 to 200 nucleotides in length, to lower concentrations of large fragments of up to thousands kilobases.

### Cancer

Methods herein may provide for early detection of cancer or for detection of non-metastatic cancer. Staging of cancer may be dependent on cancer type where each cancer type has its own classification system. Examples of cancer staging or classification systems are described in more detail below.

TABLE 1
Colon Cancer Primary Tumor (T)

| | |
|---|---|
| TX | Primary tumor cannot be assessed |
| T0 | No evidence of primary tumor |
| Tis | Carcinoma in situ: intraepithelial or intramucosal carcinoma |
| | (involvement of lamina propria with no extension through |
| | the muscularis mucosa) |
| T1 | Tumor invades submucosa (through the muscularis mucosa but |
| | not into the muscularis propria) |
| T2 | Tumor invades muscularis propria |
| T3 | Tumor invades through the muscularis propria into the |
| | pericolorectal tissues |
| T4 | Tumor invades the visceral peritoneum or invades or adheres to |
| | adjacent organ or structure |
| T4a | Tumor invades through the visceral peritoneum (including gross |
| | perforation of the bowel through tumor and continuous invasion |
| | of tumor through areas of inflammation to the surface of the |
| | visceral peritoneum) |
| T4b | Tumor directly invades or is adherent to other organs or |
| | structures |

Colon Cancer Regional Lymph Notes (N)

| | |
|---|---|
| NX | Regional lymph nodes cannot be assessed |
| N0 | No regional lymph node metastasis |
| N1 | Metastasis in 1-3 regional lymph nodes (tumor in lymph nodes |
| | measuring ≥0.2 mm) or any number of tumor deposits are |
| | present, and all identifiable nodes are negative |
| N1a | Metastasis in 1 regional lymph node |
| N1b | Metastasis in 2-3 regional lymph nodes |
| N1c | Tumor deposit(s) in the subserosa, mesentery, or |
| | nonperitonealized, pericolic, or perirectal/mesorectal tissues |
| | without regional nodal metastasis |
| N2 | Metastasis in 4 or more lymph nodes |
| N2a | Metastasis in 4-6 regional lymph nodes |
| N2b | Metastasis in 7 or more regional lymph nodes |

Colon Cancer Distant Metastasis (M)

| | |
|---|---|
| M0 | No distant metastasis by imaging or other studies, no evidence of |
| | tumor in distant sites or organs. (This category is not assigned |
| | by pathologists.) |
| M1 | Metastasis to one or more distant sites or organs or peritoneal |
| | metastasis |
| M1a | Metastasis confined to 1 organ or site (egg, liver, lung, ovary, |
| | nonregional node) without peritoneal metastasis |
| M1b | Metastasis to two or more sites or organs without peritoneal |
| | metastasis |
| M1c | Metastasis to the peritoneal surface alone or with other site or |
| | organ metastases |

TABLE 2
Colon Cancer Anatomic stage/prognostic groups

| Stage | T | N | M | Dukes | MAC |
|---|---|---|---|---|---|
| 0 | Tis | N0 | M0 | — | — |
| I | T1 | N0 | M0 | A | A |
| | T2 | N0 | M0 | A | B1 |
| IIA | T3 | N0 | M0 | B | B2 |
| IIB | T4a | N0 | M0 | B | B2 |
| IIC | T4b | N0 | M0 | B | B3 |
| IIIA | T1-T2 | N1/N1c | M0 | C | C1 |
| | T1 | N2a | M0 | C | C1 |
| IIIB | T3-T4a | N1/N1c | M0 | C | C2 |
| | T2-T3 | N2a | M0 | C | C1/C2 |
| | T1-T2 | N2b | M0 | C | C1 |
| IIIC | T4a | N2a | M0 | C | C2 |
| | T3-T4a | N2b | M0 | C | C2 |
| | T4b | N1-N2 | M0 | C | C3 |
| IVA | Any T | Any N | M1a | — | — |
| IVB | Any T | Any N | M1b | — | — |
| IVC | Any T | Any N | M1c | — | — |

TABLE 3
Malignant Melanoma Primary Tumor (T)

| | |
|---|---|
| TX | Primary tumor cannot be assessed (i.e. curettaged melanoma) |
| T0 | No evidence of primary tumor |
| Tis | Melanoma in situ |
| T1 | Thickness ≤1.0 mm |
| | T1a: <0.8 mm without ulceration |
| | T1b: <0.8 mm with ulceration, or 0.8-1.0 mm with or without |
| | ulceration |
| T2 | Thickness >1.0-2.0 mm |
| | T2a: Without ulceration |
| | T2b: With ulceration |
| T3 | Thickness >2.0-4.0 mm |
| | T3a: Without ulceration |
| | T3b: With ulceration |
| T4 | Thickness >4.0 mm |
| | T4a: Without ulceration |
| | T4b: With ulceration |

Malignant Melanoma Regional Lymph Notes (N)

| | |
|---|---|
| NX | Regional lymph nodes cannot be assessed |
| N0 | No regional metastasis detected |
| N1 | One tumor-involved lymph node or in-transit, satellite, and/or |
| | microsatellite metastases with no tumor-involved nodes |
| | N1a: One clinically occult (i.e., detected by sentinel lymph node |
| | biopsy [SLNB]; no in-transit, satellite, or microsatellite metastases |
| | N1b: One clinically detected; no in-transit, satellite, or |
| | microsatellite metastases |
| | N1c: No regional lymph node disease; in-transit, satellite, |
| | and/or microsatellite metastases found |
| N2 | Two or three tumor-involved nodes; or in-transit, satellite, or |
| | microsatellite metastases |
| | N2a: Two or three clinically occult (i.e., detected by SLNB); no |
| | in-transit, satellite, or microsatellite metastases |
| | N2b: Two or three clinically detected; no in-transit, satellite, |
| | or microsatellite metastases |
| | N2c: One clinically occult or clinically detected; in-transit, satellite, |
| | and/or microsatellite metastases found |
| N3 | ≥4 tumor-involved nodes or in-transit, satellite, and/or microsatellite |
| | metastases with ≥2 tumor-involved nodes or any number of matted |
| | nodes without or with in-transit, satellite, and/or microsatellite |
| | metastases |
| | N3a: ≥4 clinically occult (i.e., detected by SLNB); no in-transit, |
| | satellite, or microsatellite metastases |
| | N3b: ≥4, at least one of which was clinically detected, or presence |
| | of any matted nodes; no in-transit, satellite, or microsatellite |
| | metastases |
| | N3c: ≥2 clinically occur or clinically detected and/or presence of |
| | any matted nodes, with presence of in-transit, satellite, and/or |
| | microsatellite metastases |

Malignant Melanoma Distant Metastasis (M)

| | |
|---|---|
| M0 | No detectable evidence of distant metastases |
| M1a | Metastases to skin, soft tissue (including muscle), and/or |
| | nonregional lymph nodes |
| M1b | Lung metastasis, with or without M1a involvement |
| M1c | Distant metastasis to non-central nervous system (CNS) visceral |
| | sites with or without M1a or M1b involvement |
| M1d | Distant metastasis to CNS, with or without M1a or M1b |
| | involvement |

TABLE 4
Malignant Melanoma Anatomic stage/prognostic groups

| Stage | T | N | M |
|---|---|---|---|
| 0 | Tis | N0 | M0 |
| IA | T1a | N0 | M0 |
| IB | T1b | N0 | M0 |
| | T2a | N0 | M0 |
| IIA | T2b | N0 | M0 |
| | T3a | N0 | M0 |
| IIB | T3b | N0 | M0 |
| | T4a | N0 | M0 |
| IIC | T4b | N0 | M0 |
| III | Any T, Tis | N1, N2, or N3 | M0 |
| IV | Any T | Any N | M1 |

TABLE 5
Hepatocellular Carcinoma Primary tumor (T)

| | |
|---|---|
| TX | Primary tumor cannot be assessed |
| T0 | No evidence of primary tumor |
| T1 | Solitary tumor 2 cm without vascular invasion |
| T1a | Solitary tumor <2 cm |
| T1b | Solitary tumor >2 cm without vascular invasion |
| T2 | Solitary tumor >2 cm with vascular invasion; or |
| | multiple tumors, non >5 cm |
| T3 | Multiple tumors, at least one of which is >5 cm |
| T4 | Single tumor or tumors of any size involving a major |
| | branch of the portal vein or hepatic vein, or tumor(s) |
| | with direct invasion of adjacent organs other than the |
| | gallbladder or with perforation of visceral peritoneum |

Hepatocellular Carcinoma Regional Lymph Nodes (N)

| | |
|---|---|
| NX | Regional lymph node(s) cannot be assessed |
| N0 | No regional lymph node metastasis |
| N1 | Regional lymph node metastasis |

Hepatocellular Carcinoma Distant Metastasis (M)

| | |
|---|---|
| M0 | No distant metastasis |
| M1 | Distant metastasis |

TABLE 6
Hepatocellular Carcinoma Anatomic stage/prognostic
groups

| Stage | T | N | M |
|---|---|---|---|
| IA | T1a | N0 | M0 |
| IB | T1b | N0 | M0 |
| II | T2 | N0 | M0 |
| IIIA | T3 | N0 | M0 |
| IIIB | T4 | N0 | M0 |
| IVA | Any T | N1 | M0 |
| IVB | Any T | Any N | M1 |

TABLE 7
Hepatocellular Carcinoma Histologic grade

| | |
|---|---|
| GX | Grade cannot be accessed |
| G1 | Well differentiated |
| G2 | Moderately differentiated |
| G3 | Poorly differentiated |
| G4 | Undifferentiated |

TABLE 8
Barcelona-Clinic Liver Cancer staging system

| | Performance | | Okuda | |
|---|---|---|---|---|
| Stage | Status | Tumor Stage | Stage | Liver function |
| A: Early | | | | |
| HCC | | | | |
| Al | 0 | Single, <5 cm | I | No portal |
| | | | | hypertension, |
| | | | | normal bilirubin |
| A2 | 0 | Single, <5 cm | I | Portal |
| | | | | hypertension, |
| | | | | normal bilirubin |
| A3 | 0 | Single, <5 cm | I | Portal |
| | | | | hypertension, |
| | | | | normal bilirubin |
| A4 | 0 | 3 tumors, <3 cm | I-II | Child-Pugh A-B |
| Stage B: | 0 | Large, | I-II | Child-Pugh A-B |
| Intermediate | | multinodular | | |
| HCC | | | | |
| Stage C: | 1-2 | Vascular invasion | I-II | Child-Pugh A-B |
| Advanced | | or extrahepatic | | |
| HCC | | spread | | |
| Stage D: | 3-4 | Any | I-II | Child-Pugh C |
| End-Stage | | | | |
| HCC | | | | |

TABLE 9
Ishak Fibrosis score

| Architectural Change | Score |
|---|---|
| No fibrosis | 0 |
| Fibrous expansion of some portal areas, with or | 1 |
| without short fibrous septa | |
| Fibrous expansion of most portal areas, with or | 2 |
| without short fibrous septa | |
| Fibrous expansion of portal areas with occasional | 3 |
| portal-to-portal bridging | |
| Fibrous expansion of portal areas with marked | 4 |
| bridging as well as portal-central | |
| Marked bridging (portal-to-portal and/or portal- | 5 |
| central) with occasional nodule (incomplete | |
| cirrhosis) | |
| Cirrhosis, probable or definite | 6 |

TABLE 10
Gastric Cancer Primary tumor (T)

| | |
|---|---|
| TX | Primary tumor cannot be assessed |
| T0 | No evidence of primary tumor |
| Tis | Carcinoma in situ: intraepithelial tumor without invasion of the |
| | lamina propria |
| T1 | Tumor invades lamina propria, muscularis mucosae, or |
| | submucosa |
| T1a | Tumor invades lamina propria or muscularis mucosae |
| T1b | Tumor invades submucosa |
| T2 | Tumor invades muscularis propria |
| T3 | Tumor penetrates subserosal connective tissue without invasion |
| | of visceral peritoneum or adjacent structures. |
| T4 | Tumor invades serosa (visceral peritoneum) or adjacent structures |
| T4a | Tumor invades serosa (visceral peritoneum) |
| T4b | Tumor invades adjacent structures |

Regional Lymph Nodes (N)

| | |
|---|---|
| NX | Regional lymph node(s) cannot be assessed |
| N0 | No regional lymph node metastasis |
| N1 | Metastasis in 1-2 regional lymph nodes |
| N2 | Metastasis in 3-6 regional lymph nodes |
| N3 | Metastasis in seven or more regional lymph nodes |
| N3a | Metastasis in 7-15 regional lymph nodes |
| N3b | Metastasis in 16 or more regional lymph nodes |

Distant Metastasis (M)

| | |
|---|---|
| M0 | No distant metastasis |
| M1 | Distant metastasis |

TABLE 11
Gastric Cancer Clinical stage/prognostic groups (cTNM)

| Stage | T | N | M |
|---|---|---|---|
| 0 | Tis | N0 | M0 |
| I | T1 | N0 | M0 |
| | T2 | N0 | M0 |
| IIA | T1 | N1, N2, N3 | M0 |
| | T2 | N1, N2, N3 | M0 |
| IIB | T3 | N0 | M0 |
| | T4 | N0 | M0 |
| III | T | N1, N2, N3 | M0 |
| | T4a | N1, N2, N3 | M0 |
| IVA | Any T | Any N | M0 |
| IVB | Any T | Any N | M1 |

TABLE 12
Gastric Cancer Pathological stage (pTNM)

| Stage | T | N | M |
|---|---|---|---|
| 0 | Tis | N0 | M0 |
| I | T1 | N0 | M0 |
| | T1 | N1 | M0 |
| IB | T2 | N0 | M0 |
| | T1 | N2 | M0 |
| II A | T2 | N1 | M0 |
| | T3 | N0 | M0 |
| | T1 | N3 | M0 |
| | T2 | N2 | M0 |

TABLE 13
Gastric Cancer Post-neoadjuvant therapy
staging and overall survival (ypTNM)

| | | | | 3-year | 5-year |
|---|---|---|---|---|---|
| Stage | T | N | M | survival (%) | survival (%) |
| I | T1, T2 | N0 | M0 | 81.4 | 76.5 |
| | T1 | N1 | M0 | | |
| | T1 | N2, N3 | M0 | | |
| | T2 | N1, N2 | M0 | | |
| II | T3 | N0, N1 | M0 | 54.8 | 46.3 |
| | T4a | N0 | M0 | | |
| | T2 | N3 | M0 | | |
| | T3 | M2, N3 | M0 | | |
| III | T4a | N1, N2, N3 | M0 | | |
| | T4b | N0, N1, N2, | M0 | 28.8 | 18.3 |
| | | N3 | | | |
| IV | Any T | Any N | M1 | 10.2 | 5.7 |

TABLE 14
Esophageal Cancer Primary tumor (T)

| | |
|---|---|
| TX | Primary tumor cannot be assessed |
| T0 | No evidence of primary tumor |
| Tis | High-grade dysplasia,* defined as malignant cells confined |
| | by the basement membrane |
| T1 | Tumor invades lamina propria, muscularis mucosae, or |
| | submucosa |
| T1a | Tumor invades lamina propria or muscularis mucosae |
| T1b | Tumor invades submucosa |
| T2 | Tumor invades muscularis propria |
| T3 | Tumor invades adventitia |
| T4 | Tumor invades adjacent structures |
| T4a | Resectable tumor invading pleura, pericardium, azygos vein, |
| | diaphragm or peritoneum |
| T4b | Unresectable tumor invading other adjacent structures, such |
| | as the aorta, vertebral body, and trachea |

Esophageal Cancer Regional Lymph Nodes (N)

| | |
|---|---|
| NX | Regional lymph node(s) cannot be assessed |
| N0 | No regional lymph node metastasis |
| N1 | Metastasis in 1-2 regional lymph nodes |
| N2 | Metastasis in 3-6 regional lymph nodes |
| N3 | Metastasis in 7 or more regional lymph nodes |

Esophageal Cancer Distant Metastasis (M)

| | |
|---|---|
| M0 | No distant metastasis |
| M1 | Distant metastasis |

TABLE 15
Esophageal Cancer Histologic grade
Histologic grade (G)

| | |
|---|---|
| GX | Grade cannot be assessed-stage grouping as G1 |
| G1 | Well differentiated |
| G2 | Moderately differentiated |
| G3 | Poorly differentiated or undifferentiated* |

TABLE 16
Squamous cell carcinoma location

| X | Location unknown |
|---|---|
| Upper | Cervical esophagus to lower border of azygos vein |
| Middle | Lower border of azygos vein to lower border of inferior |
| | pulmonary vein |
| Lower | Lower border of inferior pulmonary vein to stomach, |
| | including gastroesophageal junction |

TABLE 17
Esophageal Cancer Clinical stage groups

| | | | |
|---|---|---|---|
| Stage Group | cT | cN | cM |

Squamous cell carcinoma

| | | | |
|---|---|---|---|
| 0 | Tis | N0 | M0 |
| I | T1 | N0-1 | M0 |
| | T2 | N0-1 | M0 |
| II | T3 | N0 | M0 |
| | T3 | N1 | M0 |
| III | T1-3 | N2 | M0 |
| | T4 | N0-2 | M0 |
| IVA | T1-4 | N3 | M0 |
| IVB | T1-4 | N0-3 | M1 |

Adenocarcinoma

| | | | |
|---|---|---|---|
| 0 | Tis | N0 | M0 |
| I | T1 | N0 | M0 |
| IIA | T1 | N1 | M0 |
| IIB | T2 | N0 | M0 |
| | T2 | N1 | M0 |
| III | T3-4a | N0-1 | M0 |
| | T1-4a | N2 | M0 |
| IVA | T4b | N0-2 | M0 |
| | T1-4 | N3 | M0 |
| IVB | T1-4 | N0-3 | M1 |

TABLE 18
Pathologic stage groups

| | | | | | |
|---|---|---|---|---|---|
| Stage Group | pT | pN | pM | Grade | Location |

Squamous cell carcinoma

| | | | | | |
|---|---|---|---|---|---|
| 0 | Tis | N0 | M0 | N/A | Any |
| IA | T1a | N0 | M0 | G-1, X | Any |
| | T1b | N0 | M0 | G1-3, X | Any |
| IB | T1a | N0 | M0 | G2-3 | Any |
| | T2 | N0 | M0 | G1 | Any |
| | T2 | N0 | M0 | G2-3, X | Any |
| IIA | T3 | N0 | M0 | Any | Lower |
| | T34 | N0 | M0 | G1 | Upper/middle |
| | T3 | N0 | M0 | G2-3 | Upper/middle |
| | T3 | N0 | M0 | GX | Any |
| IIB | T3 | N0 | M0 | Any | X |
| | T1 | N1 | M0 | Any | Any |
| IIIA | T1 | N2 | M0 | Any | Any |
| | T2 | N1 | M0 | Any | Any |
| | T4a | N0-1 | M0 | Any | Any |
| IIIB | T3 | N1 | M0 | Any | Any |
| | T2-3 | N2 | M0 | Any | Any |
| | T4a | N2 | M0 | Any | Any |
| IVA | T4b | N0-2 | M0 | Any | Any |
| | T1-4 | N3 | M0 | Any | Any |
| IVB | T1-4 | N0-3 | M1 | Any | Any |

Adenocarcinoma

| | | | | |
|---|---|---|---|---|
| 0 | Tis | N0 | M0 | N/A |
| IA | T1a | N0 | M0 | G1, X |
| IB | T1a | N0 | M0 | G2 |
| | T1b | N0 | M0 | G1-2, X |
| | T1 | N0 | M0 | G3 |
| IC | T2 | N0 | M0 | G1-2 |
| IIA | T2 | N0 | M0 | G3, X |
| | T1 | Ni | M0 | Any |
| IIB | T3 | N0 | M0 | Any |
| | T1 | N2 | M0 | Any |
| IIIA | T2 | Ni | M0 | Any |
| | T4a | N0-1 | M0 | Any |
| IIIB | T3 | Ni | M0 | Any |
| | T2-3 | N2 | M0 | Any |
| IVA | T4a | N2 | M0 | Any |
| | T4b | N0-2 | M0 | Any |
| | T1-4 | N3 | M0 | Any |
| | R1-4 | N0-3 | M1 | Any |

TABLE 19
Postneoadjuvant therapy staging

| | | | |
|---|---|---|---|
| Stage Group | ypT | ypN | ypM |

Squamous cell carcinoma

| | | | |
|---|---|---|---|
| I | T0-2 | N0 | M0 |
| II | T3 | N0 | M0 |
| IIIA | T0-2 | N1 | M0 |
| | T4a | N0 | M0 |
| IIIB | T3 | N1 | M0 |
| | T0-3 | N2 | M0 |
| | T4a | N1-2, X | M0 |
| IVA | T4b | N0-2 | M0 |
| | T1-4 | N3 | M0 |
| IVB | T1-4 | N0-3 | M1 |

TABLE 20
Endometrial Cancer Primary Tumor (T)

| | FIGO | |
|---|---|---|
| TNM | stages | Surgical-pathologic findings |
| TX | | Primary tumor cannot be assessed |
| T0 | | No evidence of primary tumor |
| Tis | | Carcinoma in situ (preinvasive carcinoma) |
| T1 | I | Tumor confined to corpus uteri |
| T1a | IA | Tumor linked to endometrium or invades less than |
| | | one half of the myometrium |
| T1b | IB | Tumor invades one half or more of the myometrium |
| T2 | II | Tumor invades stromal connective tissue of the |
| | | cervix but does not extend beyond uterus** |
| T3a | IIIA | Tumor involves serosa and/or adnexa (direct |
| | | extension or metastasis) |
| T3b | IIIB | Vaginal involvement (direct extension or metastasis) |
| | | or parametrial involvement |
| | IIIC | Metastases to pelvic and/or para-aortic lymph nodes |
| | IV | Tumor invades bladder mucosa and/or bowel |
| | | mucosa, and/or distant metastases |
| T4 | IVA | Tumor invades bladder mucosa and/or bowel |
| | | mucosa (bullous edema is not sufficient to classify |
| | | a tumor as T4) |

Endometrial Cancer Regional Lymph Nodes (N)

| | | |
|---|---|---|
| NX | | Regional lymph nodes cannot be assessed |
| N0 | | No regional lymph node metastasis |
| N1 | IIIC1 | Regional lymph node metastasis to pelvic lymph nodes |
| N2 | IIIC2 | Regional lymph node metastasis to para-aortic |
| | | lymph nodes, with or without positive pelvic lymph |
| | | nodes |

Endometrial Cancer Distant Metastasis

| | |
|---|---|
| M0 | No distant metastasis |
| M1 | Distant metastasis (includes metastasis to inguinal |
| | lymph nodes, intraperitoneal M1 IVB disease, or |
| | lung, liver, or bone metastases; it excludes metastasis |
| | to para-aortic lymph nodes, vagina, pelvic serosa, or |
| | adnexa) |

TABLE 21
Non-Small Cell Lung Cancer Primary tumor (T)

| | |
|---|---|
| TX | Primary tumor cannot be assessed, or tumor is proven by the presence of malignant cells in |
| | sputum or bronchial washings but not visualized by imaging or bronchoscopy |
| T0 | No evidence of primary tumor |
| Tis | Carcinoma in situ |
| | Squamous cell carcinoma in situ (SCIS) |
| | Adenocarcinoma in situ (AIS): adenocarcinoma with pure lepidic pattern, ≤3 cm in |
| | greatest dimension |
| T1 | Tumor ≤3 cm in greatest dimension, surrounded by lung or visceral pleura, without |
| | bronchoscopic evidence of invasion more proximal than the lobar bronchus (i.e., not in the |
| | main bronchus) |
| T1mi | Minimally invasive adenocarcinoma: adenocarcinoma (≤3 cm in greatest dimension) with |
| | a predominantly lepidic pattern and ≤5 mm invasion in greatest dimension |
| T1a | Tumor ≤ 1 cm in greatest dimension. A superficial, spreading tumor of any size whose |
| | invasive component is limited to the bronchial wall and may extend proximal to the main |
| | bronchus also is classified as T1a, but those tumors are uncommon. |
| T1b | Tumor > 1 cm but ≤2 cm in greatest dimension |
| T1c | Tumor > 2 cm but ≤3 cm in greatest dimension |
| T2 | Tumor > 3 cm but ≤5 cm or having any of the following features: |
| | Involves the main bronchus regardless of distance to the carina, but without |
| | involvement of the carina |
| | Invades visceral pleura (PL1 or PL2) |
| | Associated with atelectasis or obstructive pneumonitis extending to the hilar |
| | region, involving part or all of the lung |
| | T2 tumors with these features are classified as T2a if ≤4 cm or if the size cannot be |
| | determined and T2b if >4 cm but ≤5 cm |
| T2a | Tumor > 3 cm but ≤4 cm in greatest dimension |
| T2b | Tumor > 4 cm but ≤5 cm in greatest dimension |
| T3 | Tumor > 5 cm but ≤7 cm in greatest dimension or directly invading any of the following: |
| | parietal pleural (PL3), chest wall (including superior sulcus tumors), phrenic nerve, |
| | parietal pericardium; or separate tumor nodule(s) in the same lobe as the primary |
| T4 | Tumor > 7 cm or tumor of any size that invades one or more of the following: diaphragm, |
| | mediastinum, heart, great vessels, trachea, recurrent laryngeal nerve, esophagus, vertebral |
| | body, or carina; or separate tumor nodule(s) in an ipsilateral lobe different from that of the |
| | primary |

Non-Small Cell Lung Cancer Regional lymph nodes (N)

| | |
|---|---|
| NX | Regional lymph nodes cannot be assessed |
| N0 | No regional node metastasis |
| N1 | Metastasis in ipsilateral peribronchial and/or ipsilateral hilar lymph nodes and |
| | intrapulmonary nodes, including involvement by direct extension |
| N2 | Metastasis in ipsilateral mediastinal and/or subcarinal lymph node(s) |
| N3c | Metastasis in the contralateral mediastinal, contralateral hilar, ipsilateral or contralateral |
| | scalene, or supraclavicular lymph node(s) |

Non-Small Cell Lung Cancer Distant metastasis (M)

| | |
|---|---|
| M0 | No distant metastasis |
| M1 | Distant metastasis |
| M1a | Separate tumor nodule(s) in a contralateral lobe tumor; tumor with pleural or pericardial |
| | nodules or malignant pleural or pericardial effusion. Most pleural (pericardial) effusion |
| | with lung cancer is a result of the tumor. In a few patients, however, multiple microscopic |
| | examinations of pleural (pericardial) fluid are negative for tumor, and the fluid is |
| | nonbloody and not an exudate. If these elements and clinical judgment dictate that the |
| | effusion is not related to the tumor, the effusion should be excluded as a staging descriptor. |
| M1b | Single extrathoracic metastasis in a single organ and involvement of a single nonregional |
| | node |
| M1c | Multiple extrathoracic metastases in a single organ or in multiple organs |

TABLE 22
Non-Small Cell Lung Cancer Anatomic stage/prognostic groups

| Stage | T | N | M |
|---|---|---|---|
| 0 | Tis | N0 | M0 |
| | T1mi | N0 | M0 |
| IA1 | T1a | N0 | M0 |
| IA2 | T1b | N0 | M0 |
| IA3 | T1c | N0 | M0 |
| IB | T2a | N0 | M0 |
| IIA | T2b | N0 | M0 |
| IIB | T1a | N1 | M0 |
| | T1b | N1 | M0 |
| | T1c | N1 | M0 |
| | T2a | N1 | M0 |
| | T2b | N1 | M0 |
| | T3 | N0 | M0 |
| | T1a | N2 | M0 |
| | T1b | N2 | M0 |
| | T1c | N2 | M0 |
| | T2a | N2 | M0 |
| IIIA | T2b | N2 | M0 |
| | T3 | N1 | M0 |
| | T4 | N0 | M0 |
| | T4 | N1 | M0 |
| IIIB | T1a | N3 | M0 |
| | T1b | N3 | M0 |
| | T1c | N3 | M0 |
| | T2a | N3 | M0 |
| | T2b | N3 | M0 |
| | T3 | N2 | M0 |
| | T4 | N2 | M0 |
| | T3 | N3 | M0 |
| IIIC | T4 | N3 | M0 |
| IVA | T Any | N Any | M1a |
| T Any | | N Any | M1b |
| IVB | T Any | N Any | M1c |

TABLE 23
Small Cell Lung Cancer Primary tumor (T)

| | |
|---|---|
| TX | Primary tumor cannot be assessed, or tumor is proven by the presence of malignant cells |
| | in sputum or bronchial washings but not visualized by imaging or bronchoscopy |
| TC | No evidence of primary tumor |
| Tis | Carcinoma in situ |
| | Squamous cell carcinoma in situ (SCIS) |
| | Adenocarcinoma in situ (AIS): adenocarcinoma with pure lepidic pattern, ≤3 cm in |
| | greatest dimension |
| T1 | Tumor ≤ 3 cm in greatest dimension, surrounded by lung or visceral pleura, without |
| | bronchoscopic evidence of invasion more proximal than the lobar bronchus (i.e., not in |
| | the main bronchus) |
| T1mi | Minimally invasive adenocarcinoma: adenocarcinoma (≤3 cm in greatest dimension) with |
| | a predominantly lepidic pattern and ≤5 mm invasion in greatest dimension |
| T1a | Tumor ≤ 1 cm in greatest dimension. A superficial, spreading tumor of any size whose |
| | invasive component is limited to the bronchial wall and may extend proximal to the main |
| | bronchus also is classified as T1a, but those tumors are uncommon. |
| T1b | Tumor > 1 cm but ≤2 cm in greatest dimension |
| T1c | Tumor > 2 cm but ≤3 cm in greatest dimension |
| T2 | Tumor > 3 cm but ≤5 cm or having any of the following features: |
| | Involves the main bronchus regardless of distance to the carina, but without |
| | involvement of the carina |
| | Invades visceral pleura (PL1 or PL2) |
| | Associated with atelectasis or obstructive pneumonitis extending to the hilar |
| | region, involving part or all of the lung |
| | T2 tumors with these features are classified as T2a if ≤4 cm or if the size cannot be |
| | determined and T2b if >4 cm but ≤5 cm |
| T2a | Tumor > 3 cm but ≤4 cm in greatest dimension |
| T2b | Tumor > 4 cm but ≤5 cm in greatest dimension |
| T3 | Tumor > 5 cm but ≤7 cm in greatest dimension or directly invading any of the following: |
| | parietal pleural (PL3), chest wall (including superior sulcus tumors), phrenic nerve, |
| | parietal pericardium; or separate tumor nodule(s) in the same lobe as the primary |
| T4 | Tumor > 7 cm or tumor of any size that invades one or more of the following: |
| | diaphragm, mediastinum, heart, great vessels, trachea, recurrent laryngeal nerve, |
| | esophagus, vertebral body, or carina; or separate tumor nodule(s) in an ipsilateral lobe |
| | different from that of the primary |

Small Cell Lung Cancer Regional lymph nodes (N)

| | |
|---|---|
| NX | Regional lymph nodes cannot be assessed |
| N0 | No regional lymph node metastasis |
| N1 | Metastasis to ipsilateral peribronchial and/or ipsilateral hilar lymph nodes and |
| | intrapulmonary nodes, including involvement by direct extension |
| N2 | Metastases in ipsilateral mediastinal and/or subcarinal lymph node(s) |
| N3 | Metastasis in contralateral mediastinal, contralateral hilar, ipsilateral or contralateral |
| | scalene, or supraclavicular lymph node(s) |

Small Cell Lung Cancer Distant metastasis (M)

| | |
|---|---|
| M0 | No distant metastasis |
| M1 | Distant metastases |
| M1a | Separate tumor nodule(s) in a contralateral lobe tumor; tumor with pleural or pericardial |
| | nodules or malignant pleural or pericardial effusion. Most pleural (pericardial) effusion |
| | with lung cancer is a result of the tumor. In a few patients, however, multiple micro- |
| | scopic examinations of pleural (pericardial) fluid are negative for tumor, and the fluid is |
| | nonbloody and not an exudate. If these elements and clinical judgment dictate that the |
| | effusion is not related to the tumor, the effusion should be excluded as a staging |
| | descriptor. |
| M1b | Single extrathoracic metastasis in a single organ and involvement of a single nonregional |
| | node |
| M1c | Multiple extrathoracic metastases in a single organ or in multiple organs |

TABLE 24
Small Cell Lung Cancer Anatomic stage/prognostic groups

| | | | |
|---|---|---|---|
| Stage | T | N | M |

Limited disease

| | | | |
|---|---|---|---|
| 0 | Tis | N0 | M0 |
| | T1mi | N0 | M0 |
| IA1 | T1a | N0 | M0 |
| IA2 | T1b | N0 | M0 |
| IA3 | T1c | N0 | M0 |
| IB | T2a | N0 | M0 |
| IIA | T2b | N0 | M0 |
| IIB | T1a | N1 | M0 |
| | T1b | N1 | M0 |
| | T1c | N1 | M0 |
| | T2a | N1 | M0 |
| | T2b | N1 | M0 |
| | T3 | N0 | M0 |
| | T1a | N2 | M0 |
| | T1b | N2 | M0 |
| | T1c | N2 | M0 |
| IIIA | T2a | N2 | M0 |
| | T2b | N2 | M0 |
| | T3 | N1 | M0 |
| | T4 | N0 | M0 |
| | T4 | N1 | M0 |
| IIIB | T1a | N3 | M0 |
| | T1b | N3 | M0 |
| | T1c | N3 | M0 |
| | T2a | N3 | M0 |
| | T2b | N3 | M0 |
| | T3 | N2 | M0 |
| | T4 | N2 | M0 |
| IIIC | T3 | N3 | M0 |

Extensive disease

| | | | |
|---|---|---|---|
| IVA | T Any | N Any | M1a |
| | T Any | N Any | M1b |
| IVB | T Any | N Any | M1c |

TABLE 25
Breast Cancer Primary tumor (T)

| | |
|---|---|
| TX | Primary tumor cannot be assessed |
| T0 | No evidence of primary tumor |
| Tis | Carcinoma in situ |
| Tis (DCIS) | Ductal carcinoma in situ |
| Tis | Paget disease of the nipple NOT associated with invasive carcinoma and/or carcinoma in |
| (Paget) | situ (DCIS) in the underlying breast parenchyma. Carcinomas in the breast parenchyma |
| | associated with Paget disease are categorized on the basis of the size and characteristics |
| | of the parenchymal disease, although the presence of Paget disease should still be noted |
| T1 | Tumor ≤ 20 mm in greatest dimension |
| T1mi | Tumor ≤ 1 mm in greatest dimension |
| T1a | Tumor > 1 mm but ≤5 mm in greatest dimension (round any measurement >1.0-1.9 |
| | mm to 2 mm) |
| T1b | Tumor > 5 mm but ≤10 mm in greatest dimension |
| T1c | Tumor > 10 mm but ≤20 mm in greatest dimension |
| T2 | Tumor > 20 mm but ≤50 mm in greatest dimension |
| T3 | Tumor > 50 mm in greatest dimension |
| T4 | Tumor of any size with direct extension to the chest wall and/or to the skin (ulceration or |
| | skin nodules), not including invasion of dermis alone |
| T4a | Extension to chest wall, not including only pectoralis muscle adherence/invasion |
| T4b | Ulceration and/or ipsilateral satellite nodules and/or edema (including peaud'orange) of |
| | the skin, which do not meet the criteria for inflammatory carcinoma |
| T4c | Both T4a and T4b |
| T4d | Inflammatory carcinoma |

Breast Cancer Regional lymph nodes (N)
Clinical

| | |
|---|---|
| cNX | Regional lymph nodes cannot be assessed (e.g., previously removed) |
| cN0 | No regional lymph node metastasis (on imaging or clinical examination) |
| cN1 | Metastasis to movable ipsilateral level I, II axillary lymph node(s) |
| cN1mi | Micrometastases (approximately 200 cells, larger than 0.2 mm, but none larger than 2.0 |
| | mm) |
| cN2 | Metastases in ipsilateral level I, II axillary lymph nodes that are clinically fixed or |
| | matted; or in ipsilateral internal mammary nodes in the absence of clinically evident |
| | axillary lymph node metastases |
| cN2a | Metastases in ipsilateral level I, II axillary lymph nodes fixed to one another (matted) |
| | or to other structures |
| cN2b | Metastases only in ipsilateral internal mammary nodes and in the absence of axillary |
| | lymph node metastases |
| cN3 | Metastases in ipsilateral infraclavicular (level III axillary) lymph node(s), with or without |
| | level I, II axillary node involvement, or in ipsilateral internal mammary lymph node(s) |
| | with level I, II axillary lymph node metastasis; or metastases in ipsilateral supraclavicular |
| | lymph node(s), with or without axillary or internal mammary lymph node involvement |
| cN3a | Metastasis in ipsilateral infraclavicular lymph node(s) |
| cN3b | Metastasis in ipsilateral internal mammary lymph node(s) and axillary lymph node(s) |
| cN3c | Metastasis in ipsilateral supraclavicular lymph node(s) |

Breast Cancer Pathologic (pN)

| | |
|---|---|
| pNX | Regional lymph nodes cannot be assessed (for example, previously removed, or not |
| | removed for pathologic study) |
| pN0 | No regional lymph node metastasis identified histologically, or isolated tumor cell |
| | clusters (ITCs) only. Note: ITCs are defined as small clusters of cells ≤ 0.2 mm, or |
| | single tumor cells, or a cluster of <200 cells in a single histologic cross-section; ITCs |
| | may be detected by routine histology or by immunohistochemical (IHC) methods; |
| | nodes containing only ITCs are excluded from the total positive node count for |
| | purposes of N classification but should be included in the total number of nodes |
| | evaluated |
| pN0(i) | No regional lymph node metastases histologically, negative IHC |
| pN0(i+) | ITCs only in regional lymph node(s) |
| pN0(mol−) | No regional lymph node metastases histologically, negative molecular findings (reverse |
| | transcriptase polymerase chain reaction [RT-PCR]) |
| pN0(mol+) | Positive molecular findings by RT-PCR; no ITCs detected |
| pN1 | Micrometastases; or metastases in 1-3 axillary lymph nodes and/or in internal mammary |
| | nodes; and/or in clinically negative internal mammary nodes with micrometastases or |
| | macrometastases by sentinel lymph node biopsy |
| pN1mi | Micrometastases (200 cells, >0.2 mm but none >2.0 mm) |
| pN1a | Metastases in 1-3 axillary lymph nodes (at least 1 metastasis > 2.0 mm) |
| pN1b | Metastases in ipsilateral internal mammary lymph nodes, excluding ITCs, detected by |
| | sentinel lymph node biopsy |
| pN1c | Metastases in 1-3 axillary lymph nodes and in internal mammary sentinel nodes (i.e., |
| | pN1a and pN1b combined) |
| pN2 | Metastases in 4-9 axillary lymph nodes; or positive ipsilateral internal mammary lymph |
| | nodes by imaging in the absence of axillary lymph node metastases |
| pN2a | Metastases in 4-9 axillary lymph nodes (at least 1 tumor deposit > 2.0 mm) |
| pN2b | Clinically detected*1 metastases in internal mammary lymph nodes with or without |
| | microscopic confirmation; with pathologically negative axillary lymph nodes |
| pN3 | Metastases in ≥10 axillary lymph nodes; or in infraclavicular (level III axillary) lymph |
| | nodes; or positive ipsilateral internal mammary lymph nodes by imaging in the presence |
| | of one or more positive level I, II axillary lymph nodes; or in >3 axillary lymph nodes |
| | and micrometastases or macrometastases by sentinel lymph node biopsy in clinically |
| | negative ipsilateral internal mammary lymph nodes; or in ipsilateral supraclavicular |
| | lymph nodes |
| pN3a | Metastases in >10 axillary lymph nodes (at least 1 tumor deposit > 2.0 mm); or |
| | metastases to the infraclavicular (level III axillary lymph) nodes |
| pN3b | pN1a or pN2a in the presence of cN2b (positive internal mammary nodes by imaging) |
| | or pN2a in the presence of pN1b |
| pN3c | Metastases in ipsilateral supraclavicular lymph nodes |

Breast Cancer Distant metastasis (M)

| | |
|---|---|
| M0 | No clinical or radiographic evidence of distant metastasis |
| cM0(i+) | No clinical or radiographic evidence of distant metastases in the presence of tumor cells |
| | or deposits no larger than 0.2 mm detected microscopically or by molecular techniques |
| | in circulating blood, bone marrow, or other nonregional nodal tissue in a patient without |
| | symptoms or signs of metastases |
| cM1 | Distant metastases detected by clinical and radiographic approaches |
| pM1 | Any histologically proven metastases in distant organs; or if in non-regional nodes, |
| | metastases > 0.2 mm |

TABLE 26
Breast Cancer Histologic grade (G)

| | |
|---|---|
| GX | Grade cannot be assessed |
| G1 | Low combined histologic grade (favorable) |
| G2 | Intermediate combined histologic grade (moderately favorable) |
| G3 | High combined histologic grade (unfavorable) |

TABLE 27
Breast Cancer Anatomic stage/prognostic groups

| Stage | T | N | M |
|---|---|---|---|
| 0 | Tis | N0 | M0 |
| IA | T1 | N0 | M0 |
| IB | T0 | N1mi | M0 |
| | T1 | N1mi | M0 |
| IIA | T0 | N1 | M0 |
| | T1 | N1 | M0 |
| | T2 | N0 | M0 |
| IIB | T2 | N1 | M0 |
| | T3 | N0 | M0 |
| IIIA | T0 | N2 | M0 |
| | T1 | N2 | M0 |
| | T2 | N2 | M0 |
| | T3 | N1 | M0 |
| | T3 | N2 | M0 |
| IIIB | T4 | N0 | M0 |
| | T4 | N1 | M0 |
| | T4 | N2 | M0 |
| IIIC | Any T | N3 | M0 |
| IV | Any T | Any N | M1 |

Methods provided herein may allow for early detection cancer or for detection of non-metastatic cancer. Examples of cancers that may be detected in accordance with a method disclosed herein include, without limitation, Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloblastoma, Medulloepithelioma, Melanoma, Melanoma, Meningioma, Merkel Cell Carcinoma, Mesothelioma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Multiple myeloma, Mycosis Fungoides, Mycosis fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral Cancer, Oral cancer, Oropharyngeal Cancer, Osteosarcoma, Osteosarcoma, Ovarian Cancer, Ovarian cancer, Ovarian Epithelial Cancer, Ovarian Germ Cell Tumor, Ovarian Low Malignant Potential Tumor, Paget's disease of the breast, Pancoast tumor, Pancreatic Cancer, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, Wilms' tumor, and combinations thereof.

### Partial List of Numbered Embodiments

The disclosure herein is further clarified in reference to a partial list of numbered embodiments as follows. Embodiment 1. A method of detecting a non-metastatic cancer in a subject, the method comprising: (a) obtaining a sample comprising a plurality of cell-free deoxyribonucleic acid (cfDNA) polynucleotides of the subject; (b) using at least a subset of the plurality of cfDNA polynucleotides or derivatives thereof to measure a total cfDNA fragment size distribution for the plurality of cfDNA polynucleotides of the subject; (c) computer processing the total cfDNA fragment size distribution for the plurality of cfDNA polynucleotides of the subject with the total cfDNA fragment size distribution for the plurality of cfDNA polynucleotides from a healthy control; and (d) classifying the subject as having an increased risk of non-metastatic cancer when, based at least in part on a result of (c), the total cfDNA fragment size distribution shows an increase in fragments having sizes of up to 170 bases in the subject as compared to the healthy control. Embodiment 2. The method of embodiment 1, wherein (b) is performed by sequencing the at least the subset of the plurality of cfDNA polynucleotides or derivatives thereof. Embodiment 3. The method of embodiment 1 or embodiment 2, further comprising, prior to (b), preparing a single stranded deoxyribonucleic acid (DNA) library from the plurality of cfDNA polynucleotides of the subject. Embodiment 4. The method of embodiment 1 or embodiment 2, further comprising, prior to (b), preparing a double stranded DNA library from the plurality of cfDNA polynucleotides of the subject. Embodiment 5. The method of any one of embodiments 1 to 4, further comprising, prior to (b): (a) circularizing individual cfDNA polynucleotides of the plurality of cfDNA polynucleotides of the subject to form a plurality of circular polynucleotides; (b) amplifying the circular polynucleotides to yield amplified polynucleotides; (c) sequencing the amplified polynucleotides to produce a plurality of sequencing reads; and (d) determining a length for each individual cfDNA polynucleotide of the plurality of cfDNA polynucleotides of the subject. Embodiment 6. The method of any one of embodiments 1 to 5, wherein the total cfDNA fragment size distribution shows at least a 0.5% increase in fragments having sizes of 50 bases to 170 bases in the subject as compared to the healthy control. Embodiment 7. The method of any one of embodiments 1 to 6, wherein the total cfDNA fragment size distribution shows at least a 1% increase in fragments having sizes of 50 bases to 170 bases in the subject as compared to the healthy control. Embodiment 8. The method of any one of embodiments 1 to 7, wherein the total cfDNA fragment size distribution shows at least a 10% increase in fragments having sizes of 50 bases to 170 bases in the subject as compared to the healthy control. Embodiment 9. The method of any one of embodiments 1 to 8, wherein the method comprises classifying the subject as having an increased risk of non-metastatic cancer when the total cfDNA fragment size distribution shows an increase in fragments having sizes of 50 bases to 125 bases in the subject as compared to the healthy control. Embodiment 10. The method of any one of embodiments 1 to 9, wherein the total cfDNA fragment size distribution shows at least a 0.5% increase in fragments having sizes of 50 bases to 125 bases in the subject as compared to the healthy control. Embodiment 11. The method of any one of embodiments 1 to 10, wherein the total cfDNA fragment size distribution shows at least a 1% increase in fragments having sizes of 50 bases to 125 bases in the subject as compared to the healthy control. Embodiment 12. The method of any one of embodiments 1 to 11, wherein the total cfDNA fragment size distribution shows at least a 10% increase in fragments having sizes of 50 bases to 125 bases in the subject as compared to the healthy control. Embodiment 13. The method of any one of embodiments 1 to 12, wherein the method comprises classifying the subject as having an increased risk of non-metastatic cancer when the total cfDNA fragment size distribution shows an increase in fragments having sizes of 50 bases to 115 bases in the subject as compared to the healthy control. Embodiment 14. The method of any one of embodiments 1 to 13, wherein the total cfDNA fragment size distribution shows at least a 0.5% increase in fragments having sizes of 50 bases to 115 bases in the subject as compared to the healthy control. Embodiment 15. The method of any one of embodiments 1 to 14, wherein the total cfDNA fragment size distribution shows at least a 1% increase in fragments having sizes of 50 bases to 115 bases in the subject as compared to the healthy control. Embodiment 16. The method of any one of embodiments 1 to 15, wherein the total cfDNA fragment size distribution shows at least a 10% increase in fragments having sizes of 50 bases to 115 bases in the subject as compared to the healthy control. Embodiment 17. The method of any one of embodiments 1 to 16, wherein the method comprises classifying the subject as having an increased risk of non-metastatic cancer when the total cfDNA fragment size distribution shows an increase in fragments having sizes of 50 bases to 105 bases in the subject as compared to the healthy control. Embodiment 18. The method of any one of embodiments 1 to 17, wherein the total cfDNA fragment size distribution shows at least a 0.5% increase in fragments having sizes of 50 bases to 105 bases in the subject as compared to the healthy control. Embodiment 19. The method of any one of embodiments 1 to 18, wherein the total cfDNA fragment size distribution shows at least a 1% increase in fragments having sizes of 50 bases to 105 bases in the subject as compared to the healthy control. Embodiment 20. The method of any one of embodiments 1 to 19, wherein the total cfDNA fragment size distribution shows at least a 10% increase in fragments having sizes of 50 bases to 105 bases in the subject as compared to the healthy control. Embodiment 21. The method of any one of embodiments 1 to 20, wherein the subject does not have a diagnosis of metastatic cancer. Embodiment 22. The method of any one of embodiments 1 to 21, wherein the subject has a tumor burden of less than 10%. Embodiment 23. The method of any one of embodiments 1 to 22, wherein the cancer is selected from the group consisting of colon cancer, non-small cell lung cancer, small cell lung cancer, breast cancer, hepatocellular carcinoma, liver cancer, skin cancer, malignant melanoma, endometrial cancer, esophageal cancer, gastric cancer, ovarian cancer, pancreatic cancer, and brain cancer. Embodiment 24. The method of any one of embodiments 1 to 23, wherein the method does not comprise isolating tumor cfDNA polynucleotides from total cfDNA polynucleotides. Embodiment 25. The method of any one of embodiments 1 to 24, wherein the non-metastatic cancer is stage 0, stage 1, stage 2, or stage 3. Embodiment 26. The method of any one of embodiments 1 to 25, further comprising recommending administration of a chemotherapy to the subject. Embodiment 27. The method of any one of embodiments 1 to 26, further comprising recommending additional cancer monitoring to the subject. Embodiment 28. The method of any one of embodiments 1 to 27, wherein (d) comprises classifying the subject as having an increased risk of non-metastatic cancer when the total cfDNA fragment size distribution shows an increase in fragments having sizes of 50 bases up to 170 in the subject as compared to the healthy control. Embodiment 29. The method of any one of embodiments 1 to 28, further comprising enriching the plurality of cfDNA polynucleotides for one or more target sequences. Embodiment 30. The method of embodiment 5, further comprising enriching said plurality of cfDNA polynucleotides for one or more target sequences prior to circularizing said individual cfDNA polynucleotides. Embodiment 31. The method of embodiment 5, further comprising enriching said circular polynucleotides for one or more target sequences prior to amplifying said circular polynucleotides. Embodiment 32. The method of embodiment 5, further comprising enriching said circular polynucleotides for one or more target sequences during amplifying said circular polynucleotides. Embodiment 33. The method of embodiment 5, further comprising enriching said amplified polynucleotides for one or more target sequences prior to said sequencing. Embodiment 34. The method of any one of embodiments 30 to 33, wherein said enriching is performed with aid of a targeted primer(s) or capture probe(s). Embodiment 35. The method of embodiment 34, wherein said plurality of sequencing reads are processed using a sequence(s) of said targeted primer(s) or capture probe(s). Embodiment 36. A method of measuring a cfDNA size distribution in a blood sample from a subject, the method comprising: (a) obtaining a sample comprising a plurality of cell-free deoxyribonucleic acid (cfDNA) polynucleotides from the blood sample of the subject; (b) using at least a subset of the plurality of cfDNA polynucleotides or derivatives thereof to measure a total cfDNA fragment size distribution for the plurality of cfDNA polynucleotides of the subject; (c) computer processing the total cfDNA fragment size distribution for the plurality of cfDNA polynucleotides of the subject with the total cfDNA fragment size distribution for the plurality of cfDNA polynucleotides from a healthy control; and (d) classifying the subject as having an increased risk of non-metastatic cancer when, based at least in part on a result of (c), the total cfDNA fragment size distribution shows an increase in fragments having sizes of 50 bases to 170 bases in the subject as compared to the healthy control. Embodiment 37. The method of embodiment 36, wherein the plurality of cfDNA polynucleotides comprises less than 10% circulating tumor DNA (ctDNA). Embodiment 38. The method of embodiment 36 or embodiment 37, wherein the plurality of cfDNA polynucleotides comprises less than 5% circulating tumor DNA (ctDNA). Embodiment The method of any one of embodiments 36 to 38, wherein the plurality of cfDNA polynucleotides comprises less than 2% circulating tumor DNA (ctDNA). Embodiment 40. The method of any one of embodiments 36 to 39, wherein the plurality of cfDNA polynucleotides comprises less than 1% circulating tumor DNA (ctDNA). Embodiment 41. The method of any one of embodiments 36 to 40, further comprising, prior to (b), preparing a single stranded DNA library from the plurality of cfDNA polynucleotides of the subject. Embodiment 42. The method of any one of embodiments 36 to 40, further comprising, prior to (b), preparing a double stranded DNA library from the plurality of cfDNA polynucleotides of the subject. Embodiment 43. The method of any one of embodiments 36 to 42, further comprising, prior to (b): (a) circularizing individual cfDNA polynucleotides of the plurality of cfDNA polynucleotides of the subject to form a plurality of circular polynucleotides; (b) amplifying the circular polynucleotides; (c) sequencing the amplified polynucleotides to produce a plurality of sequencing reads; and (d) determining a length for each individual cfDNA polynucleotide of the plurality of cfDNA polynucleotides of the subject. Embodiment 44. The method of any one of embodiments 36 to 43, wherein the total cfDNA fragment size distribution shows at least a 0.5% increase in fragments having sizes of 50 bases to 170 bases in the subject as compared to the healthy control. Embodiment 45. The method of any one of embodiments 36 to 44, wherein the total cfDNA fragment size distribution shows at least a 1% increase in fragments having sizes of 50 bases to 170 bases in the subject as compared to the healthy control. Embodiment 46. The method of any one of embodiments 36 to 45, wherein the total cfDNA fragment size distribution shows at least a 10% increase in fragments having sizes of 50 bases to 170 bases in the subject as compared to the healthy control. Embodiment 47. The method of any one of embodiments 36 to 46, wherein the total method comprises classifying the subject as having an increased risk of non-metastatic cancer when the total cfDNA fragment size distribution shows an increase in fragments having sizes of 50 bases to 125 bases in the subject as compared to the healthy control. Embodiment 48. The method of any one of embodiments 36 to 47, wherein the total cfDNA fragment size distribution shows at least a 0.5% increase in fragments having sizes of 50 bases to 125 bases in the subject compared to the healthy control. Embodiment 49. The method of any one of embodiments 36 to 48, wherein the total cfDNA fragment size distribution shows at least a 1% increase in fragments having sizes of 50 bases to 125 bases in the subject as compared to the healthy control. Embodiment 50. The method of any one of embodiments 36 to 49, wherein the total cfDNA fragment size distribution shows at least a 10% increase in fragments having sizes of 50 bases to 125 bases in the subject as compared to the healthy control. Embodiment 51. The method of any one of embodiments 36 to 50, wherein the method comprises classifying the subject as having an increased risk of non-metastatic cancer when the total cfDNA fragment size distribution shows an increase in fragments having sizes of 50 bases to 115 bases in the subject as compared to the healthy control. Embodiment 52. The method of any one of embodiments 36 to 51, wherein the total cfDNA fragment size distribution shows at least a 0.5% increase in fragments having sizes of 50 bases to 115 bases in the subject as compared to the healthy control. Embodiment 53. The method of any one of embodiments 36 to 52, wherein the total cfDNA fragment size distribution shows at least a 1% increase in fragments having sizes of 50 bases to 115 bases in the subject as compared to the healthy control. Embodiment 54. The method of any one of embodiments 36 to 53, wherein the total cfDNA fragment size distribution shows at least a 10% increase in fragments having sizes of 50 bases to 115 bases in the subject as compared to the healthy control. Embodiment 55. The method of any one of embodiments 36 to 54, wherein the method comprises classifying the subject as having an increased risk of non-metastatic cancer when the total cfDNA fragment size distribution shows an increase in fragments having sizes of 50 bases to 105 bases in the subject as compared to the healthy control. Embodiment 56. The method of any one of embodiments 36 to 55, wherein the total cfDNA fragment size distribution shows at least a 0.5% increase in fragments having sizes of 50 bases to 105 bases in the subject as compared to the healthy control. Embodiment 57. The method of any one of embodiments 36 to 56, wherein the total cfDNA fragment size distribution shows at least a 1% increase in fragments having sizes of 50 bases to 105 bases in the subject as compared to the healthy control. Embodiment 58. The method of any one of embodiments 36 to 57, wherein the total cfDNA fragment size distribution shows at least a 10% increase in fragments having sizes of 50 bases to 105 bases in the subject as compared to the healthy control. Embodiment 59. The method of any one of embodiments 36 to 58, wherein the subject does not have a diagnosis of metastatic cancer. Embodiment 60. The method of any one of embodiments 36 to 59, wherein the subject has a tumor burden of less than 10%. Embodiment 61. The method of any one of embodiments 36 to 60, wherein the cancer is selected from the group consisting of colon cancer, non-small cell lung cancer, small cell lung cancer, breast cancer, hepatocellular carcinoma, liver cancer, skin cancer, malignant melanoma, endometrial cancer, esophageal cancer, gastric cancer, ovarian cancer, pancreatic cancer, and brain cancer. Embodiment 62. The method of any one of embodiments 36 to 61, wherein the method does not comprise isolating tumor cfDNA polynucleotides from total cfDNA polynucleotides. Embodiment 63. The method of any one of embodiments 36 to 62, wherein the non-metastatic cancer is stage 0, stage 1, stage 2, or stage 3. Embodiment 64. The method of any one of embodiments 36 to 63, further comprising recommending administration of a chemotherapy to the subject. Embodiment 65. The method of any one of embodiments 36 to 64, further comprising recommending additional cancer monitoring to the subject. Embodiment 66. The method of any one of embodiments 36 to 65, further comprising, prior to (b), enriching the plurality of cfDNA polynucleotides for one or more target sequences. Embodiment 67. The method of embodiment 43, further comprising enriching said plurality of cfDNA polynucleotides for one or more target sequences prior to circularizing said individual cfDNA polynucleotides. Embodiment 68. The method of embodiment 43, further comprising enriching said circular polynucleotides for one or more target sequences prior to amplifying said circular polynucleotides. Embodiment 69. The method of embodiment 43, further comprising enriching said circular polynucleotides for one or more target sequences during amplifying said circular polynucleotides. Embodiment 70. The method of embodiment 43, further comprising enriching said amplified polynucleotides for one or more target sequences prior to said sequencing. Embodiment 71. The method of any one of embodiments 67 to 70, wherein said enriching is performed with aid of a targeted primer(s) or capture probe(s). Embodiment 72. The method of embodiment 71, wherein said plurality of sequencing reads are processed using a sequence(s) of said targeted primer(s) or capture probe(s). Embodiment 73. A method of detecting a tumor in a subject, the method comprising: (a) obtaining a sample comprising a plurality of cell-free deoxyribonucleic acid (cfDNA) polynucleotides from a blood sample of the subject; (b) using at least a subset of the plurality of cfDNA polynucleotides or derivatives thereof to measure a total cfDNA fragment size distribution for the plurality of cfDNA polynucleotides of the subject; (c) computer processing the total cfDNA fragment size distribution for the plurality of cfDNA polynucleotides of the subject with the total cfDNA fragment size distribution for the plurality of cfDNA polynucleotides from a healthy control; and (d) detecting a tumor when, based at least in part on a result of (c), the total cfDNA fragment size distribution shows an increase in fragments having sizes of 50 bases to 170 bases in the subject as compared to the healthy control. Embodiment 74. The method of embodiment 73, further comprising, based at least in part on a result of (c), classifying the subject as having an increased risk of non-metastatic cancer when the total cfDNA fragment size distribution shows an increase in fragments having sizes of 50 bases to 170 bases in the subject as compared to the healthy control. Embodiment 75. The method of embodiment 73 or embodiment 74, further comprising, prior to (b), preparing a single stranded DNA library from the plurality of cfDNA polynucleotides of the subject. Embodiment 76. The method of embodiment 73 or embodiment 74, further comprising, prior to (b), preparing a double stranded DNA library from the plurality of cfDNA polynucleotides of the subject. Embodiment 77. The method of any one of embodiments 73 to 76, further comprising, prior to (b): (a) circularizing individual cfDNA polynucleotides of the plurality of cfDNA polynucleotides of the subject to form a plurality of circular polynucleotides; (b) amplifying the circular polynucleotides; (c) sequencing the amplified polynucleotides to produce a plurality of sequencing reads; and (d) determining a length for each individual cfDNA polynucleotide of the plurality of cfDNA polynucleotides of the subject. Embodiment 78. The method of any one of embodiments 73 to 77, wherein the total cfDNA fragment size distribution shows at least a 0.5% increase in fragments having sizes of 50 bases to 170 bases in the subject as compared to the healthy control. Embodiment 79. The method of any one of embodiments 73 to 78, wherein the total cfDNA fragment size distribution shows at least a 1% increase in fragments having sizes of 50 bases to 170 bases in the subject as compared to the healthy control. Embodiment 80. The method of any one of embodiments 73 to 79, wherein the total cfDNA fragment size distribution shows at least a 10% increase in fragments having sizes of 50 bases to 170 bases in the subject as compared to the healthy control. Embodiment 81. The method of any one of embodiments 73 to 80, wherein the method comprises classifying the subject as having an increased risk of non-metastatic cancer when the total cfDNA fragment size distribution shows an increase in fragments having sizes of 50 bases to 125 bases in the subject as compared to the healthy control. Embodiment 82. The method of any one of embodiments 73 to 81, wherein the total cfDNA fragment size distribution shows at least a 0.5% increase in fragments having sizes of 50 bases to 125 bases in the subject as compared to the healthy control. Embodiment 83. The method of any one of embodiments 73 to 82, wherein the total cfDNA fragment size distribution shows at least a 1% increase in fragments having sizes of 50 bases to 125 bases in the subject as compared to the healthy control. Embodiment 84. The method of any one of embodiments 73 to 83, wherein the total cfDNA fragment size distribution shows at least a 10% increase in fragments having sizes of 50 bases to 125 bases in the subject as compared to the healthy control. Embodiment 85. The method of any one of embodiments 73 to 84, wherein the method comprises classifying the subject as having an increased risk of non-metastatic cancer when the total cfDNA fragment size distribution shows an increase in fragments having sizes of 50 bases to 115 bases in the subject as compared to the healthy control. Embodiment 86. The method of any one of embodiments 73 to 85, wherein the total cfDNA fragment size distribution shows at least a 0.5% increase in fragments having sizes of 50 bases to 115 bases in the subject as compared to the healthy control. Embodiment 87. The method of any one of embodiments 73 to 86, wherein the total cfDNA fragment size distribution shows at least a 1% increase in fragments having sizes of 50 bases to 115 bases in the subject as compared to the healthy control. Embodiment 88. The method of any one of embodiments 73 to 87, wherein the total cfDNA fragment size distribution shows at least a 10% increase in fragments having sizes of 50 bases to 115 bases in the subject as compared to the healthy control. Embodiment 89. The method of any one of embodiments 73 to 88, wherein the method comprises classifying the subject as having an increased risk of non-metastatic cancer when the total cfDNA fragment size distribution shows an increase in fragments having sizes of 50 bases to 105 bases in the subject as compared to the healthy control. Embodiment 90. The method of any one of embodiments 73 to 89, wherein the total cfDNA fragment size distribution shows at least a 0.5% increase in fragments having sizes of 50 bases to 105 bases in the subject as compared to the healthy control. Embodiment 91. The method of any one of embodiments 73 to 90, wherein the total cfDNA fragment size distribution shows at least a 1% increase in fragments having sizes of 50 bases to 105 bases in the subject as compared to the healthy control. Embodiment 92. The method of any one of embodiments 73 to 91, wherein the total cfDNA fragment size distribution shows at least a 10% increase in fragments having sizes of 50 bases to 105 bases in the subject as compared to the healthy control. Embodiment 93. The method of any one of embodiments 73 to 92, wherein the subject does not have a diagnosis of metastatic cancer. Embodiment 94. The method of any one of embodiments 73 to 93, wherein the subject has a tumor burden of less than 10%. Embodiment 95. The method of any one of embodiments 73 to 94, wherein the cancer is selected from the group consisting of colon cancer, non-small cell lung cancer, small cell lung cancer, breast cancer, hepatocellular carcinoma, liver cancer, skin cancer, malignant melanoma, endometrial cancer, esophageal cancer, gastric cancer, ovarian cancer, pancreatic cancer, and brain cancer. Embodiment 96. The method of any one of embodiments 73 to 95, wherein the method does not comprise isolating tumor cfDNA polynucleotides from total cfDNA polynucleotides. Embodiment 97. The method of any one of embodiments 73 to 96, wherein the non-metastatic cancer is stage 0, stage 1, stage 2, or stage 3. Embodiment 98. The method of any one of embodiments 73 to 97, further comprising recommending administration of a chemotherapy to the subject. Embodiment 99. The method of any one of embodiments 73 to 98, further comprising recommending additional cancer monitoring to the subject. Embodiment 100. The method of any one of embodiments 73 to 99, further comprising, prior to (b) enriching the plurality of cfDNA polynucleotides for one or more target sequences. Embodiment 101. The method of embodiment 77, further comprising enriching said plurality of cfDNA polynucleotides for one or more target sequences prior to circularizing said individual cfDNA polynucleotides. Embodiment 102. The method of embodiment 77, further comprising enriching said circular polynucleotides for one or more target sequences prior to amplifying said circular polynucleotides. Embodiment 103. The method of embodiment 77, further comprising enriching said circular polynucleotides for one or more target sequences during amplifying said circular polynucleotides. Embodiment 104. The method of embodiment 77, further comprising enriching said amplified polynucleotides for one or more target sequences prior to said sequencing. Embodiment 105. The method of any one of embodiments 101 to 104, wherein said enriching is performed with aid of a targeted primer(s) or capture probe(s). Embodiment 106. The method of embodiment 105, wherein said plurality of sequencing reads are processed using a sequence(s) of said targeted primer(s) or capture probe(s). Embodiment 107. A system for detecting a non-metastatic cancer in a subject, the system comprising (a) a computer configured to receive a user request to perform a detection of non-metastatic cancer on a sample comprising a plurality of cell-free deoxyribonucleic acid (cfDNA) polynucleotides; (b) an amplification unit that performs a nucleic acid amplification reaction on at least a subset of the plurality of cfDNA polynucleotides or derivatives thereof in response to the user request to yield amplified cfDNA polynucleotides; (c) a sequencing unit that (i) sequences the amplified cfDNA polynucleotides or derivatives thereof to produce a plurality of sequencing reads; (ii) determines a length for each individual polynucleotide of the plurality of cfDNA polynucleotides of the subject; (iii) produces a total cfDNA fragment size distribution for a plurality of cfDNA polynucleotides in the sample; and (iv) computer processing the total cfDNA fragment size distribution for the plurality of cfDNA polynucleotides of the subject with the total cfDNA fragment size distribution for the plurality of cfDNA polynucleotides from a healthy control; and (d) a report generator that sends a report to a recipient, wherein the report contains results indicating a risk of non-metastatic cancer in the subject. Embodiment 108. The system of embodiment 107, wherein the system comprises an isolation system for isolating cell-free DNA (cfDNA) polynucleotides from a blood sample of the subject. Embodiment 109. The system of embodiment 107 or embodiment 108, wherein the system compares the total cfDNA fragment size distribution for the plurality of cfDNA polynucleotides of the subject to the total cfDNA fragment size distribution for the plurality of cfDNA polynucleotides from a healthy control. Embodiment 110. The system of any one of embodiments 107 to 109, wherein the system detects a non-metastatic cancer in the subject when the total cfDNA fragment size distribution shows an increase in fragments having sizes of 50 bases to 170 bases in the subject as compared to the healthy control. Embodiment 111. The system of any one of embodiments 107 to 110, wherein the report generator classifies the subject as having an increased risk of non-metastatic cancer when the total cfDNA fragment size distribution shows an increase in fragments having sizes of 50 bases to 170 bases in the subject as compared to the healthy control. Embodiment 112. The system of any one of embodiments 107 to 111, further comprising a preparation system for preparation of a single stranded DNA library from the plurality of cfDNA polynucleotides of the subject. Embodiment 113. The system of any one of embodiments 107 to 111, further comprising a preparation system for preparation of a double stranded DNA library from the plurality of cfDNA polynucleotides of the subject. Embodiment 114. The system of any one of embodiments 107 to 113, wherein the amplification reaction comprises: circularizing individual cfDNA polynucleotides of the plurality of cfDNA polynucleotides of the subject to form a plurality of circular polynucleotides; and amplifying the circular polynucleotides. Embodiment 115. The system of any one of embodiments 107 to 114, wherein the total cfDNA fragment size distribution shows at least a 0.5% increase in fragments having sizes of 50 bases to 170 bases in the subject as compared to the healthy control. Embodiment 116. The system of any one of embodiments 107 to 115, wherein the total cfDNA fragment size distribution shows at least a 1% increase in fragments having sizes of 50 bases to 170 bases in the subject as compared to the healthy control. Embodiment 117. The system of any one of embodiments 107 to 116, wherein the total cfDNA fragment size distribution shows at least a 10% increase in fragments having sizes of 50 bases to 170 bases in the subject as compared to the healthy control. Embodiment 118. The system of any one of embodiments 107 to 117, wherein the report generator classifies the subject as having an increased risk of non-metastatic cancer when the total cfDNA fragment size distribution shows an increase in fragments having sizes of 50 bases to 125 bases in the subject as compared to the healthy control. Embodiment 119. The system of any one of embodiments 107 to 118, wherein the total cfDNA fragment size distribution shows at least a 0.5% increase in fragments having sizes of 50 bases to 125 bases in the subject as compared to the healthy control. Embodiment 120. The system of any one of embodiments 107 to 119, wherein the total cfDNA fragment size distribution shows at least a 1% increase in fragments having sizes of 50 bases to 125 bases in the subject as compared to the healthy control. Embodiment 121. The system of any one of embodiments 107 to 120, wherein the total cfDNA fragment size distribution shows at least a 10% increase in fragments having sizes of 50 bases to 125 bases in the subject as compared to the healthy control. Embodiment 122. The system of any one of embodiments 107 to 121, wherein the report generator classifies the subject as having an increased risk of non-metastatic cancer when the total cfDNA fragment size distribution shows an increase in fragments having sizes of 50 bases to 115 bases in the subject as compared to the healthy control. Embodiment 123. The system of any one of embodiments 107 to 122, wherein the total cfDNA fragment size distribution shows at least a 0.5% increase in fragments having sizes of 50 bases to 115 bases in the subject as compared to the healthy control. Embodiment 124. The system of any one of embodiments 107 to 123, wherein the total cfDNA fragment size distribution shows at least a 1% increase in fragments having sizes of 50 bases to 115 bases in the subject as compared to the healthy control. Embodiment 125. The system of any one of embodiments 107 to 124, wherein the total cfDNA fragment size distribution shows at least a 10% increase in fragments having sizes of 50 bases to 115 bases in the subject as compared to the healthy control. Embodiment 126. The system of any one of embodiments 107 to 125, wherein the report generator classifies the subject as having an increased risk of non-metastatic cancer when the total cfDNA fragment size distribution shows an increase in fragments having sizes of 50 bases to 105 bases in the subject as compared to the healthy control. Embodiment 127. The system of any one of embodiments 107 to 126, wherein the total cfDNA fragment size distribution shows at least a 0.5% increase in fragments having sizes of 50 bases to 105 bases in the subject as compared to the healthy control. Embodiment 128. The system of any one of embodiments 107 to 127, wherein the total cfDNA fragment size distribution shows at least a 1% increase in fragments having sizes of 50 bases to 105 bases in the subject as compared to the healthy control. Embodiment 129. The system of any one of embodiments 107 to 128, wherein the total cfDNA fragment size distribution shows at least a 10% increase in fragments having sizes of 50 bases to 105 bases in the subject as compared to the healthy control. Embodiment 130. The system of any one of embodiments 107 to 129, wherein the subject does not have a diagnosis of metastatic cancer. Embodiment 131. The system of any one of embodiments 107 to 130, wherein the subject has a tumor burden of less than 10%. Embodiment 132. The system of any one of embodiments 107 to 131, wherein the cancer is selected from the group consisting of colon cancer, non-small cell lung cancer, small cell lung cancer, breast cancer, hepatocellular carcinoma, liver cancer, skin cancer, malignant melanoma, endometrial cancer, esophageal cancer, gastric cancer, ovarian cancer, pancreatic cancer, and brain cancer. Embodiment 133. The system of any one of embodiments 107 to 132, wherein the system does not isolate tumor cfDNA polynucleotides from total cfDNA polynucleotides. Embodiment 134. The system of any one of embodiments 107 to 133, wherein the non-metastatic cancer is stage 0, stage 1, stage 2, or stage 3. Embodiment 135. The system of any one of embodiments 107 to 134, wherein the report further contains a recommendation for administration of a chemotherapy to the subject. Embodiment 136. The system of any one of embodiments 107 to 135, wherein the report further contains a recommendation of additional cancer monitoring to the subject. Embodiment 137. The system of any one of embodiments 107 to 136, wherein the amplification unit enriches the plurality of cfDNA polynucleotides for one or more target sequences. Embodiment 138. A computer-readable medium comprising code that, upon execution by one or more computer processors, implements a method of detecting a non-metastatic cancer in a subject, the method comprising: (a) receiving a user request to perform a detection of non-metastatic cancer on a sample comprising a plurality of cell-free deoxyribonucleic acid (cfDNA) polynucleotides from a subject; (b) performing a nucleic acid amplification reaction on at least a subset of the plurality of cfDNA polynucleotides or derivatives thereof to yield amplified cfDNA polynucleotides; (c) performing a sequencing analysis comprising the steps of (i) sequencing the amplified cfDNA polynucleotides to produce a plurality of sequencing reads; (ii) determining a length for each individual polynucleotide of the plurality of cfDNA polynucleotides of the subject; (iii) producing a total cfDNA fragment size distribution for a plurality of cfDNA polynucleotides in the sample; and (iv) processing the total cfDNA fragment size distribution for the plurality of cfDNA polynucleotides of the subject with the total cfDNA fragment size distribution for the plurality of cfDNA polynucleotides from a healthy control; and (d) generating a report that contains results indicating a risk of non-metastatic cancer in the subject. Embodiment 139. The computer-readable medium of embodiment 138, wherein the method comprises isolating cell-free DNA (cfDNA) polynucleotides from a blood sample of the subject. Embodiment 140. The computer-readable medium of embodiment 138 or embodiment 139, wherein the method comprises comparing the total cfDNA fragment size distribution for the plurality of cfDNA polynucleotides of the subject to the total cfDNA fragment size distribution for the plurality of cfDNA polynucleotides from a healthy control. Embodiment 141. The computer-readable medium of any one of embodiments 138 to 140, wherein the method comprises detecting a non-metastatic cancer in the subject when the total cfDNA fragment size distribution shows an increase in fragments having sizes of 50 bases to 170 bases in the subject as compared to the healthy control. Embodiment 142. The computer-readable medium of any one of embodiments 138 to 141, wherein the method comprises classifying the subject as having an increased risk of non-metastatic cancer when the total cfDNA fragment size distribution shows an increase in fragments having sizes of 50 bases to 170 bases in the subject as compared to the healthy control. Embodiment 143. The computer-readable medium of any one of embodiments 138 to 142, further comprising preparing a single stranded DNA library from the plurality of cfDNA polynucleotides of the subject. Embodiment 144. The computer-readable medium of any one of embodiments 138 to 142, further comprising preparing a double stranded DNA library from the plurality of cfDNA polynucleotides of the subject. Embodiment 145. The computer-readable medium of any one of embodiments 138 to 144, wherein the amplification reaction comprises: circularizing individual cfDNA polynucleotides of the plurality of cfDNA polynucleotides of the subject to form a plurality of circular polynucleotides; and amplifying the circular polynucleotides. Embodiment 146. The computer-readable medium of any one of embodiments 138 to 145, wherein the total cfDNA fragment size distribution shows at least a 0.5% increase in fragments having sizes of 50 bases to 170 bases in the subject as compared to the healthy control. Embodiment 147. The computer-readable medium of any one of embodiments 138 to 146, wherein the total cfDNA fragment size distribution shows at least a 1% increase in fragments having sizes of 50 bases to 170 bases in the subject as compared to the healthy control. Embodiment 148. The computer-readable medium of any one of embodiments 138 to 147, wherein the total cfDNA fragment size distribution shows at least a 10% increase in fragments having sizes of 50 bases to 170 bases in the subject as compared to the healthy control. Embodiment 149. The computer-readable medium of any one of embodiments 138 to 148, wherein the method comprises classifying the subject as having an increased risk of non-metastatic cancer when the total cfDNA fragment size distribution shows an increase in fragments having sizes of 50 bases to 125 bases in the subject as compared to the healthy control. Embodiment 150. The computer-readable medium of any one of embodiments 138 to 149, wherein the total cfDNA fragment size distribution shows at least a 0.5% increase in fragments having sizes of 50 bases to 125 bases in the subject as compared to the healthy control. Embodiment 151. The computer-readable medium of any one of embodiments 138 to 150, wherein the total cfDNA fragment size distribution shows at least a 1% increase in fragments having sizes of 50 bases to 125 bases in the subject as compared to the healthy control. Embodiment 152. The computer-readable medium of any one of embodiments 138 to 151, wherein the total cfDNA fragment size distribution shows at least a 10% increase in fragments having sizes of 50 bases to 125 bases in the subject as compared to the healthy control. Embodiment 153. The computer-readable medium of any one of embodiments 138 to 152, wherein the method comprises classifying the subject as having an increased risk of non-metastatic cancer when the total cfDNA fragment size distribution shows an increase in fragments having sizes of 50 bases to 115 bases in the subject as compared to the healthy control. Embodiment 154. The computer-readable medium of any one of embodiments 138 to 153, wherein the total cfDNA fragment size distribution shows at least a 0.5% increase in fragments having sizes of 50 bases to 115 bases in the subject as compared to the healthy control. Embodiment 155. The computer-readable medium of any one of embodiments 138 to 154, wherein the total cfDNA fragment size distribution shows at least a 1% increase in fragments having sizes of 50 bases to 115 bases in the subject as compared to the healthy control. Embodiment 156. The computer-readable medium of any one of embodiments 138 to 155, wherein the total cfDNA fragment size distribution shows at least a 10% increase in fragments having sizes of 50 bases to 115 bases in the subject as compared to the healthy control. Embodiment 157. The computer-readable medium of any one of embodiments 138 to 156, wherein the method comprises classifying the subject as having an increased risk of non-metastatic cancer when the total cfDNA fragment size distribution shows an increase in fragments having sizes of 50 bases to 105 bases in the subject as compared to the healthy control. Embodiment 158. The computer-readable medium of any one of embodiments 138 to 157, wherein the total cfDNA fragment size distribution shows at least a 0.5% increase in fragments having sizes of 50 bases to 105 bases in the subject as compared to the healthy control. Embodiment 159. The computer-readable medium of any one of embodiments 138 to 158, wherein the total cfDNA fragment size distribution shows at least a 1% increase in fragments having sizes of 50 bases to 105 bases in the subject as compared to the healthy control. Embodiment 160. The computer-readable medium of any one of embodiments 138 to 159, wherein the total cfDNA fragment size distribution shows at least a 10% increase in fragments having sizes of 50 bases to 105 bases in the subject as compared to the healthy control. Embodiment 161. The computer-readable medium of any one of embodiments 138 to 160, wherein the subject does not have a diagnosis of metastatic cancer. Embodiment 162. The computer-readable medium of any one of embodiments 138 to 161, wherein the subject has a tumor burden of less than 10%. Embodiment 163. The computer-readable medium of any one of embodiments 138 to 162, wherein the cancer is selected from the group consisting of colon cancer, non-small cell lung cancer, small cell lung cancer, breast cancer, hepatocellular carcinoma, liver cancer, skin cancer, malignant melanoma, endometrial cancer, esophageal cancer, gastric cancer, ovarian cancer, pancreatic cancer, and brain cancer. Embodiment 164. The computer-readable medium of any one of embodiments 138 to 163, wherein the method does not comprise isolating tumor cfDNA polynucleotides from total cfDNA polynucleotides. Embodiment 165. The computer-readable medium of any one of embodiments 138 to 164, wherein the non-metastatic cancer is stage 0, stage 1, stage 2, or stage 3. Embodiment 166. The computer-readable medium of any one of embodiments 138 to 165, wherein the report further contains a recommendation for administration of a chemotherapy to the subject. Embodiment 167. The computer-readable medium of any one of embodiments 138 to 166, further comprising recommending additional cancer monitoring to the subject. Embodiment 168. The computer-readable medium of any one of embodiments 138 to 167, wherein performing an amplification reaction comprises enriching the plurality of cfDNA polynucleotides for one or more target sequences. Embodiment 169. A method of detecting a non-metastatic cancer in a subject, comprising: (a) obtaining a sample comprising a plurality of cell-free deoxyribonucleic acid (cfDNA) nucleic acid molecules of said subject; (b) using at least a subset of the plurality of cfDNA polynucleotides or derivatives thereof to measure a total cfDNA fragment size distribution for said plurality of cfDNA nucleic acid molecules; and (c) determining that said subject has or is at increased risk of having a non-metastatic cancer when, based at least in part on a result of (b), said total cfDNA fragment size distribution shows an increase in fragments as compared to a total cfDNA fragment size distribution for said plurality of nucleic acid molecules from a healthy control.

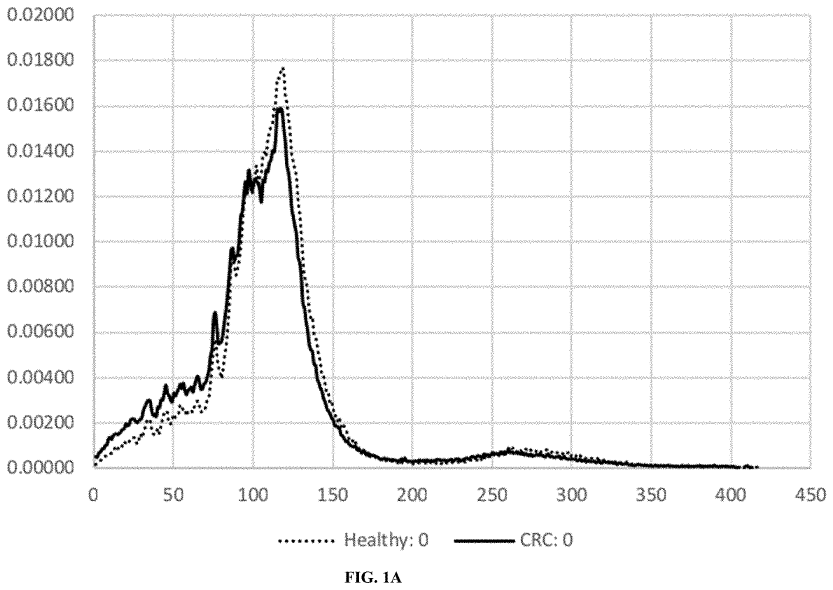

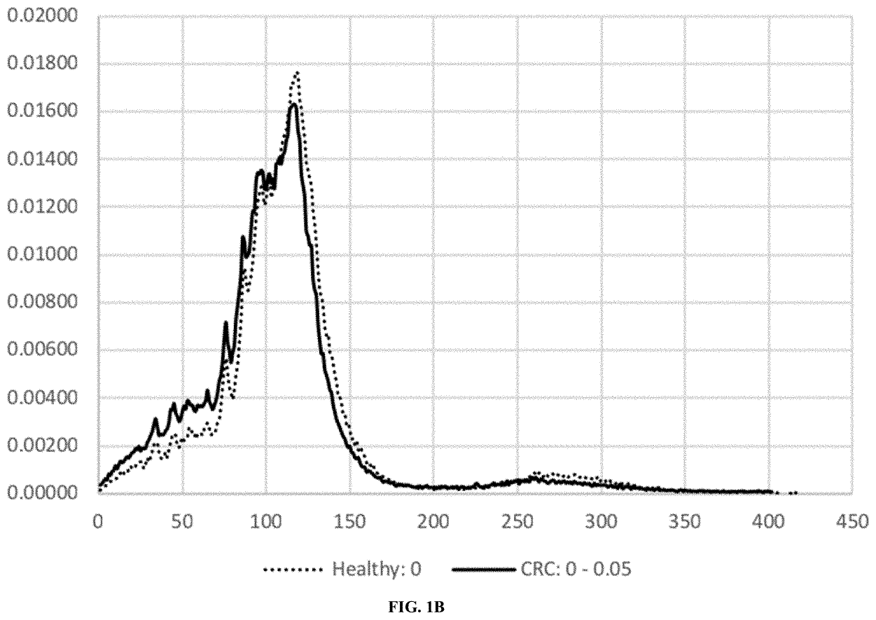

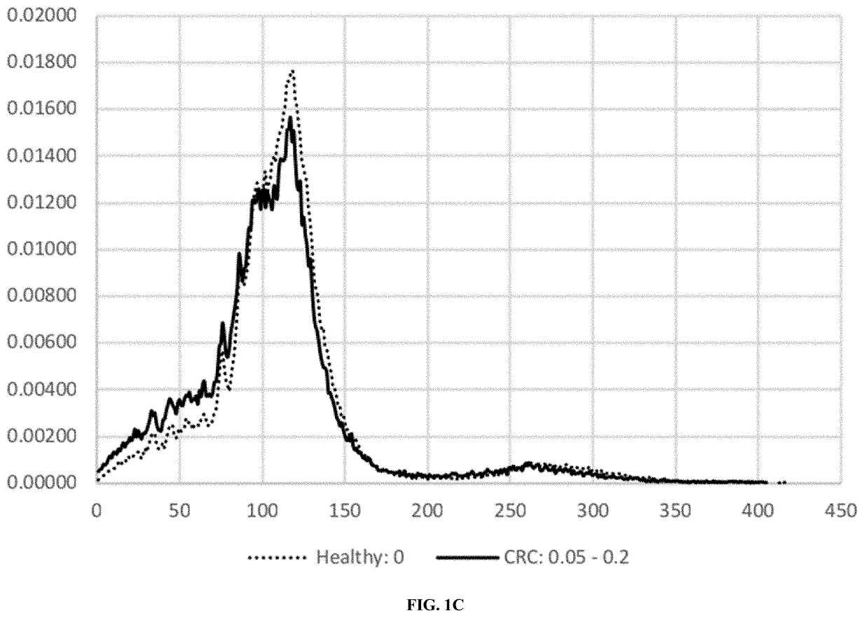

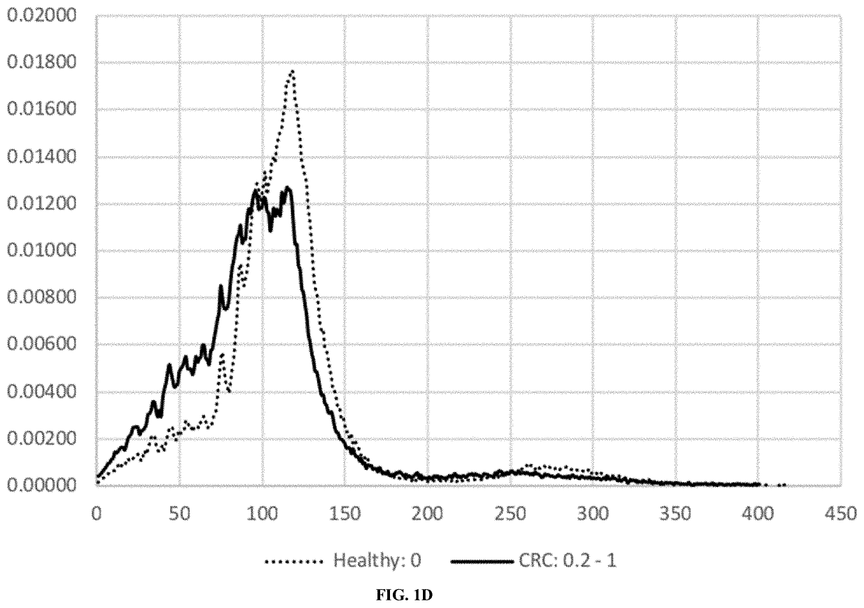

FIG. 1 shows a distribution graph of cfDNA sizes with a percentage on the Y axis and a size on the X axis. Five categories of subjects are shown here, each having a distribution. The graph shows a distribution having the lowest percentage in the small size range (i.e., 50-150 bp) comes from samples from healthy subjects. The graph then shows three distributions showing a very similar percentage, just above the healthy subject distribution in the small size range (i.e., 50-150 bp), these samples come from subjects having cancer and with an mutant allele frequency of either 0%, between 0% and 5%, or between 5% and 20%. The graph then shows a distribution having the highest percentage in the small size range (i.e., 50-150 bp) coming from samples from subjects having cancer and a mutant allele frequency greater than 20%. This figure illustrates that subjects having very early stage cancer can be identified by measuring cfDNA size.

FIG. 1A shows a distribution graph of cfDNA sizes with a percentage on the Y axis and a size on the X axis. The graph shows a distribution having the lowest percentage in the small size range (i.e., 50-150 bp) comes from samples from healthy subjects while individuals having cancer with mutant allele frequency of 0 have an increase in fragments in the small size range. This figure illustrates that subjects having very early stage cancer can be identified by measuring cfDNA size.

FIG. 1B shows a distribution graph of cfDNA sizes with a percentage on the Y axis and a size on the X axis. The graph shows a distribution having the lowest percentage in the small size range (i.e., 50-150 bp) comes from samples from healthy subjects while individuals having cancer with mutant allele frequency greater than 0 and less than 5 have an increase in fragments in the small size range. This figure illustrates that subjects having very early stage cancer can be identified by measuring cfDNA size.

FIG. 1C shows a distribution graph of cfDNA sizes with a percentage on the Y axis and a size on the X axis. The graph shows a distribution having the lowest percentage in the small size range (i.e., 50-150 bp) comes from samples from healthy subjects while individuals having cancer with mutant allele frequency greater or equal to 5 and less than 20 have an increase in fragments in the small size range. This figure illustrates that subjects having very early stage cancer can be identified by measuring cfDNA size.

FIG. 1D shows a distribution graph of cfDNA sizes with a percentage on the Y axis and a size on the X axis. The graph shows a distribution having the lowest percentage in the small size range (i.e., 50-150 bp) comes from samples from healthy subjects while individuals having cancer with mutant allele frequency of greater than or equal to 20 have an increase in fragments in the small size range. This figure illustrates that subjects having very early stage cancer can be identified by measuring cfDNA size and that more advanced cancers show a further increase in small size cfDNA fragments.

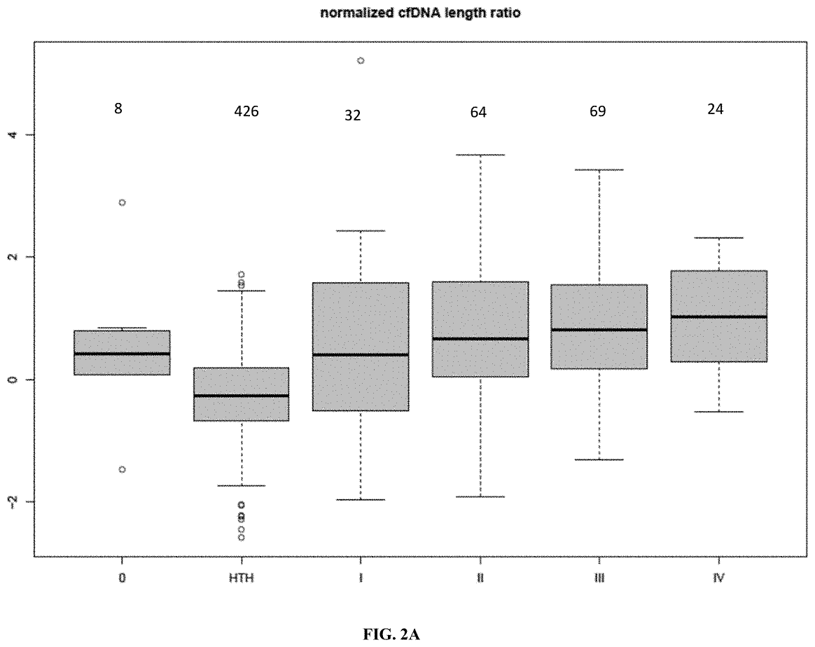

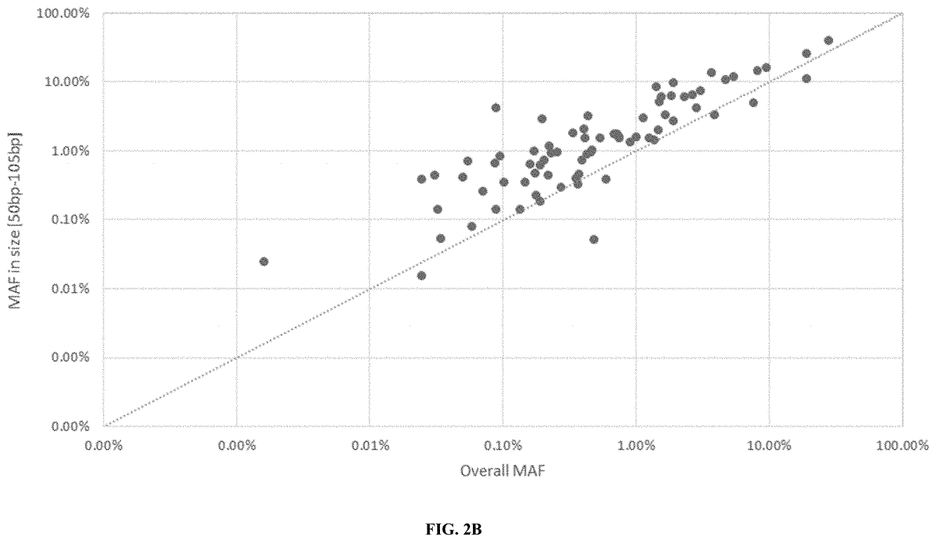

FIG. 2A shows boxplots of the normalized cfDNA fragment sizes of healthy and cancer samples from different stages including stage 0, healthy subjects, stage I, stage II, stage III, and stage IV. Each of the subjects having cancer, including early stage cancer show an increase in small size cfDNA fragments. This figure illustrates that subjects having very early stage cancer can be identified by measuring cfDNA size.

FIG. 2B shows a scatter plot of mutant allele frequency versus mutant allele frequency calculated using molecules in size range 50 bp-150 bp. MAF in small size range is higher than over MAF, indicating enrichment of ctDNA in small fragments. However, for most samples with overall MAF<1%, the MAF in small size range are still lower than 5%. Dashed line: y=x. This figure illustrates an enrichment of mutant alleles in small cfDNA fragments.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1: Obtaining cfDNA Size Distribution

Circularization of Single Strand cfDNA:

12 μl of purified cfDNA fragments is denatured by heating at 95° C. for 30 seconds and chilling on ice for 2 minutes. Then, 8 μl of ligation mix containing 2 μl of 10× CircLigase buffer, 4 μl of 5M Betaine, 1 μl of 50 mM MnCl2, and 1 μl of CircLigase II is added to the denatured DNA samples and the reactions are incubated at 60° C. for one hour.

Rolling Cycle Amplification of Circular Target Polynucleotides:

For each reaction, add 5 μl of isothermal amplification buffer, 0.75 uL of dNTP mix (25 mM each), 2 μl of 10 μM gene specific primers, 20.25 μl of water. Heat the reaction at 80° C. for 1 minute and incubated at 63° C. for 5 minutes before cooling down to 4° C. Next, add 15 units of Bst 2.0 warm start DNA polymerase to each reaction, and incubated in a thermal cycler with the following program: 4 cycles of 60° C. for 30 seconds; 70° C. for 4.5 minutes; 94° C. for 20 seconds; and 58° C. for 10 seconds.

2nd Round of PCR and Sequencing:

Purify rolling cycle amplification products by addition of 45 μl Ampure beads, following the manufacturer's instructions for the remaining wash steps. Elute in 25 μl of elution buffer. Further amplify the purified RCA products by PCR with primers containing sequencing adapters. Sequence the resulting amplification products by NGS.

NGS Data Analysis:

Align FASTQ files to a reference file containing the target sequence. Calculate cfDNA fragment size based on sequencing data. Plot cfDNA fragment size distribution.

### Example 2: Comparison of cfDNA Fragment Size from Healthy and Colorectal Cancer (CRC) Patient Samples with Different Mutant Allele Frequencies

cfDNA from 24 of healthy and 63 CRC patient blood samples were circularized and amplified as described in the protocol, using a panel of primers targeting KRAS, NRAS, PIK3CA and BRAF genes. Variants were identified after concatemer error correction and comparison with human reference genome (hg19). Overall variant allele frequencies were calculated. Samples were divided into 5 groups as following: 1. Healthy, 2. CRC with maximum mutant allele frequency=0%, 3. CRC with 0%<maximum mutant allele frequency <5%, 4. CRC with 5%≤maximum mutant allele frequency <20%, and 5. CRC with 20%≤maximum mutant allele frequency.

Average cfDNA fragment size distributions of each groups were plotted and overlaid for comparison (FIG. 1).

### Example 3: Comparison of cfDNA Fragment Size from Healthy and CRC Samples of Different Cancer Stages

cfDNA from 426 of healthy individuals, and 197 CRC patients (8 stage, 32 stage I, 64 stage II, 69 stage III, 24 stage IV) were circularized and amplified as described in the protocol, using a panel of primers targeting genes in Table 28 below.

TABLE 28
Target Genes for Analysis

| | | | | | |
|---|---|---|---|---|---|
| CTNNB1 | EGFR | FBXW7 | FGFR2 | PIK3CA | TP53 |
| AKT1 | APC | BRAF | CDKN2A | PPP2R1A | TOP2A |
| GNAS | HRAS | KRAS | NRAS | PTEN | RNF43 |

Fraction of cfDNA fragments in size range 50 bases—105 bases was calculated for each sample. The mean and the standard deviation of the fraction were calculated across all samples (both healthy and CRC). The fraction of each sample was then normalized by subtracting the mean and followed dividing with standard deviation. FIG. 2A shows boxplots of the normalized cfDNA fragment sizes of healthy and cancer samples from different stages.

Variants were identified after concatemer error correction and comparison with human reference genome (hg19). Mutant allele frequencies for molecules in size range 50 bases—105 bases were calculated and compared with the overall mutant allele frequency. (FIG. 2B).

### Example 4: Screening Non-Metastatic Cancer

An individual presents at a clinic for a routine checkup. A blood sample is taken from the individual to determine whether they have or are at risk for developing early stage cancer. Cell-free DNA (cfDNA) is isolated from the sample and then amplified prior to sequencing. The individual shows an increase of cfDNA in the size range of 50 to 170 bases indicating that the individual has early stage or non-metastatic cancer or that they are at risk of developing cancer. The individual is advised to seek care with an oncologist who can assess the state or stage of cancer. The individual is treated for the cancer and survives cancer free for 10 years.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein may be employed. It is intended that the following claims define the scope of the invention and that methods and structures within the scope of these claims and their equivalents be covered thereby.

1. A method of detecting a non-metastatic cancer in a subject, the method comprising:
(a) obtaining a sample comprising a plurality of cell-free deoxyribonucleic acid (cfDNA) polynucleotides of the subject;
(b) using at least a subset of the plurality of cfDNA polynucleotides or derivatives thereof to measure a total cfDNA fragment size distribution for the plurality of cfDNA polynucleotides of the subject;
(c) computer processing the total cfDNA fragment size distribution for the plurality of cfDNA polynucleotides of the subject with the total cfDNA fragment size distribution for the plurality of cfDNA polynucleotides from a healthy control; and
(d) classifying the subject as having an increased risk of non-metastatic cancer when, based at least in part on a result of (c), the total cfDNA fragment size distribution shows an increase in fragments having sizes of up to 170 bases in the subject as compared to the healthy control.

2. The method of claim 1, wherein (b) is performed by sequencing the at least the subset of the plurality of cfDNA polynucleotides or derivatives thereof.

3. The method of claim 1, further comprising, prior to (b), preparing a single stranded deoxyribonucleic acid (DNA) library from the plurality of cfDNA polynucleotides of the subject.

4. The method of claim 1, further comprising, prior to (b), preparing a double stranded DNA library from the plurality of cfDNA polynucleotides of the subject.

5. The method of claim 1, further comprising, prior to (b):
(a) circularizing individual cfDNA polynucleotides of the plurality of cfDNA polynucleotides of the subject to form a plurality of circular polynucleotides;
(b) amplifying the circular polynucleotides to yield amplified polynucleotides;
(c) sequencing the amplified polynucleotides to produce a plurality of sequencing reads; and
(d) determining a length for each individual cfDNA polynucleotide of the plurality of cfDNA polynucleotides of the subject.

6. The method of claim 1, wherein the total cfDNA fragment size distribution shows at least a 0.5% increase in fragments having sizes of 50 bases to 170 bases in the subject as compared to the healthy control.

7. The method of claim 1, wherein the total cfDNA fragment size distribution shows at least a 1% increase in fragments having sizes of 50 bases to 170 bases in the subject as compared to the healthy control.

8. The method of claim 1, wherein the subject does not have a diagnosis of metastatic cancer.

9. The method of claim 1, wherein the subject has a tumor burden of less than 10%.

10. The method of claim 1, wherein the cancer is selected from the group consisting of colon cancer, non-small cell lung cancer, small cell lung cancer, breast cancer, hepatocellular carcinoma, liver cancer, skin cancer, malignant melanoma, endometrial cancer, esophageal cancer, gastric cancer, ovarian cancer, pancreatic cancer, and brain cancer.

11. The method of claim 1, wherein the method does not comprise isolating tumor cfDNA polynucleotides from total cfDNA polynucleotides.

12. (canceled)

13. The method of claim 1, further comprising recommending administration of a chemotherapy to the subject.

14. The method of claim 1, further comprising recommending additional cancer monitoring to the subject.

15. The method of claim 1, further comprising enriching the plurality of cfDNA polynucleotides for one or more target sequences.

16.-19. (canceled)

20. The method of claim 15, wherein said enriching is performed with aid of a targeted primer(s) or capture probe(s).

21. The method of claim 20, wherein said plurality of sequencing reads are processed using a sequence(s) of said targeted primer(s) or capture probe(s).

22.-100. (canceled)

101. A method of detecting a non-metastatic cancer in a subject, comprising:
(a) obtaining a sample comprising a plurality of cell-free deoxyribonucleic acid (cfDNA) nucleic acid molecules of said subject;
(b) using at least a subset of the plurality of cfDNA polynucleotides or derivatives thereof to measure a total cfDNA fragment size distribution for said plurality of cfDNA nucleic acid molecules; and
(c) determining that said subject has or is at increased risk of having a non-metastatic cancer when, based at least in part on a result of (b),_said total cfDNA fragment size distribution shows an increase in fragments as compared to a total cfDNA fragment size distribution for said plurality of nucleic acid molecules from a healthy control.

102. The method of claim 101, further comprising, prior to (b), preparing a single stranded deoxyribonucleic acid (DNA) library from the plurality of cfDNA polynucleotides of the subject.

103. The method of claim 101, further comprising, prior to (b), preparing a double stranded DNA library from the plurality of cfDNA polynucleotides of the subject.

104. The method of claim 101, further comprising, prior to (b):
(a) circularizing individual cfDNA polynucleotides of the plurality of cfDNA polynucleotides of the subject to form a plurality of circular polynucleotides;
(b) amplifying the circular polynucleotides to yield amplified polynucleotides;
(c) sequencing the amplified polynucleotides to produce a plurality of sequencing reads; and
(d) determining a length for each individual cfDNA polynucleotide of the plurality of cfDNA polynucleotides of the subject.

105. The method of claim 101, wherein the total cfDNA fragment size distribution shows at least a 0.5% increase in fragments having sizes of 50 bases to 170 bases in the subject as compared to the healthy control.

106. The method of claim 101, wherein the total cfDNA fragment size distribution shows at least a 1% increase in fragments having sizes of 50 bases to 170 bases in the subject as compared to the healthy control.

107. The method of claim 101, wherein the subject does not have a diagnosis of metastatic cancer.

108. The method of claim 101, wherein the cancer is selected from the group consisting of colon cancer, non-small cell lung cancer, small cell lung cancer, breast cancer, hepatocellular carcinoma, liver cancer, skin cancer, malignant melanoma, endometrial cancer, esophageal cancer, gastric cancer, ovarian cancer, pancreatic cancer, and brain cancer.

109. The method of claim 101, wherein the method does not comprise isolating tumor cfDNA polynucleotides from total cfDNA polynucleotides.
